(19) United States
(12) Patent Application Publication
(10) Pub. No.: US 2010/0324049 A1
(43) Pub. Date: Dec. 23, 2010

(54) DIARYL KETIMINE DERIVATIVE HAVING ANTAGONISM AGAINST MELANIN-CONCENTRATING HORMONE RECEPTOR

(76) Inventors: Makoto Ando, Ebina-shi (JP); Minoru Kameda, Kashiwa-shi (JP); Hiroshi Miyazoe, Tsukuba-shi (JP); Minoru Moriya, Tsukuba-shi (JP); Etsuko Hirose, Tsuchiura-shi (JP); Takao Suzuki, Pudong (JP)

Correspondence Address: MERCK, P O BOX 2000, RAHWAY, NJ, 07065-0907, US

(21) Appl. No.: 12/676,938

(22) PCT Filed: Sep. 26, 2008
(86) PCT No.: PCT/JP2008/067406

§ 371 Date: Mar. 8, 2010

(30) Foreign Application Priority Data
Sep. 27, 2007 (JP) 2007-251356

(51) Int. Cl. A61K 31/4545; C07D 401/14; C07D 401/04; C07D 211/52; C07D 211/26; C07D 471/04; A61K 31/451; A61K 31/501; A61K 31/437; A61K 31/4985; C07D 487/04; A61K 31/5025; A61K 31/497; A61P 3/04; A61P 3/10; A61P 25/24; A61P 25/22; A61P 1/16; A61P 3/00

(52) U.S. Cl. 514/249; 546/194; 546/242; 546/247; 544/238; 546/121; 546/113; 544/410; 544/316; 514/318; 514/327; 514/252.03; 514/300; 544/350; 544/236; 514/248; 514/255.05

(57) ABSTRACT

[Problems] To provide an antagonist of a melanin-concentrating hormone receptor, which is useful as a medicine for a central nervous system disease, a cardiovascular disease or a metabolic disease.

[Means for Solving Problems] The antagonist comprises, as an active ingredient, a compound represented by the formula (I)

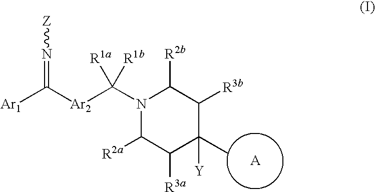

wherein R^{1a} and R^{1b} independently represent a hydrogen atom or a C₁₋₆ alkyl group; R^{2a}, R^{2b}, R^{3a} and R^{3b} independently represent a hydrogen atom, a C₁₋₆ alkyl group, or the like; Y represents H or —OH; Z represents —OR⁸, or the like; R⁸ represents a hydrogen atom, a C₁₋₆ alkyl group which may have a substituent, or the like; R^{9a} and R^{9b} independently represent a hydrogen atom, a C₁₋₆ alkyl group, or the like; Ar₁ represents an aromatic carbon ring group, or an aromatic heteroring group; Ar₂ represents a group produced by removing two hydrogen atoms from an aromatic carbon ring, or the like; and the ring group A represents an unsaturated heteroring group.

### TECHNICAL FIELD

The invention relates to novel diaryl ketimine derivatives. These compounds, which act as antagonists to melanin-concentrating hormone receptor, are useful in preventing, treating or remedying various circulatory diseases, neurological diseases, metabolic diseases, reproductive diseases, respiratory diseases, digestive tract diseases and so on.

### BACKGROUND ART

Melanin-concentrating hormone (hereafter abbreviated as “MCH”) is a cyclic peptide hormone/neuro-peptide, which was isolated for the first time from salmon hypophysis by Kawauchi, et al. in 1983 (Nature, Vol. 305, 321, 1983). It has been known that this hormone functionally antagonizes against melanin cell-stimulating hormone in fishes, thus causes concentration of melanin granules in melanophore and, in its turn, participates in body color change (International Review of Cytology, Vol. 126, 1, 1991; Trends in Endocrinology and Metabolism, Vol. 5, 120, 1994). In mammals, although MCH-containing neuron nerve cells are localized in the hypothalamus lateral field and uncertain zone, the nerve fibers thereof are projecting over a very wide scope in the brain (The Journal of Comparative Neurology, Vol. 319, 218, 1992). Therefore, it is assumed that MCH controls various central functions in living bodies.

Hypothalamus lateral field has been known as the feeding center for a long time. In recent years, moreover, there have been accumulated a number of molecular biological and pharmacological findings suggesting that MCH would participate in controlling energetic homeostasis. That is, it is reported that the expression of an MCH precursor mRNA was accelerated in the brains of model animals of hereditary obesity such as ob/ob mice, db/db mice, KKAy mice and Zucker fatty rats and fasted mice (Nature, Vol. 380, 243, 1996; Diabetes, Vol. 47, 294, 1998; Biochemical and Biophysical Research Communications, Vol. 268, 88, 2000; and Molecular Brain Research, Vol. 92, 43, 2001).

Acute ventricular administration of MCH to rats induced accelerated feeding (Nature, Vol. 380, 243, 1996) and chronic administration thereof results in obesity accompanied by overeating (Proceedings of the National Academy of Science of the United States of America, Vol. 99, 3240, 2002). Moreover, MCH precursor gene-deficient mice show reduced food intake or a rise in oxygen consumption per body weight and thus show low body weight due to decrease in body fat, compared to wild type mice (Nature, Vol. 396, 670, 1998).

On the other hand, transgenic mice with over expression of MCH precursor display obesity accompanied by overeating and insulin resistance (The Journal of Clinical Investigation, Vol. 107, 379, 2001). As a result, it is suggested that MCH is an important factor causative of obesity and participates in the onset of metabolic diseases and respiratory diseases for which obesity is a risk factor. In addition, it has been known that MCH also participates in also anxiety-causing action, epilepsy, memory/learning, diuretic action, sodium/potassium excretory action, oxytocin secretory action, reproduction and reproductive functions and so on (Peptides, Vol. 17, 171, 1996; Peptides, Vol. 18, 1095, 1997; Peptides, Vol, 15, 757, 1994; Journal of Neuroendocrinology, Vol. 8, 57, 1996; and Critical Reviews in Neurobiology, Vol. 8, 221, 1994).

MCH induces various pharmacological actions through MCH receptors which occur mainly in the central nervous system. There have been known at least two types MCH receptors, namely, type 1 receptors (MCH-1R or SLC-1) and type 2 receptors (MCH-2R or SLT) (Nature, Vol. 400, 261, 1999; Nature, Vol. 400, 265, 1999; Biochemical and Biophysical Research Communications, Vol. 261, 622, 1999; Nature Cell Biology, Vol. 1, 267, 1999; FEBS Letters, Vol. 457, 522, 1999; Biochemical and Physical Research Communications, Vol. 283, 1013, 2001; The Journal of Biological Chemistry, Vol. 276, 20125, 2001; Proceedings of the National Academy of Sciences of the United States of America, Vol. 98, 7564, 2001; Proceedings of the National Academy of Sciences of the United States of America, Vol. 98, 7576, 2001; The Journal of Biological Chemistry, Vol. 276, 34664, 2001; and Molecular Pharmacology, Vol. 60, 632 (2001)].

In particular, the pharmacological action observed on rodents is induced mainly via MCH-1R (Genomics, Vol. 79, 785, 2002). Since the chronic administration of MCH to MCH-1R gene-deficient mice causes no overeating or obesity, it is known that the energy exchange control by MCH is induced via MCH-1R. Furthermore, it is known that the deficiency of MCH-1R promotes the exercise amount of mice (Proceedings of the National Academy of Sciences of the United States of America, Vol. 99, 3240, 2002). Thus, it is strongly suggested that MCH would participate in central diseases accompanied by behavioral abnormalities, for example, attention-deficit hyperactivity disorder, schizophrenia, depression and the like (Molecular Medicine Today, Vol. 6, 43, 2000; and Trends in Neuroscience, Vol. 24, 527, 2001).

It is also reported that autoantibody against MC1-1-1R occurs in the serum of vitiligo vulgaris patients (The Journal of Clinical Investigation, Vol. 109, 923, 2002). Furthermore, the expression of MCH-1R in certain kind of cancer cells is reported. Based on the expression sites of MCH and MCH-1R in vivo, it is also suggested that MCH and MCH-1R would participate in cancer, sleeping/awaking, drug dependency and digestive disorders (Biochemical and Biophysical Research Communications, Vol. 289, 44, 2001; Neuroendocrinology, Vol. 61, 348, 1995; Endocrinology, Vol. 137, 561, 1996; and The Journal of Comparative Neurology, Vol. 435, 26, 2001).

The functions of MCH are expressed upon its binding to MCH receptors. Therefore, the expression of the actions of MCH can be inhibited by preventing MCH from binding to MCH receptors. Accordingly, it is expected that substances capable of antagonizing the binding of MCH to its receptors are useful as preventives or remedies for various diseases in which MCH participates, for example, metabolic disorders such as obesity, diabetes, hormone disorder, hyperlipidemia, gout, fatty liver, hepatitis and so on; circulatory diseases such as stenocardia, acute or congestive heart failure, myocardial infarction, coronary atherosclerosis, hypertension, renal diseases, electrolyte abnormality and so on; central/peripheral nervous system disorders such as bulimia, emotional disturbance, depression, anxiety, epilepsy, delirium, dementia, schizophrenia, attention-deficit hyperactivity disorder, memory impairment, sleep disorders, cognitive failure, dyskinesia, paresthesias, smell disorders, morphine tolerance, drug dependence, alcoholism and so on; reproductive disorders such as infertility, preterm labor, sexual dysfunction and so on; digestive disorders; respiratory disorders; cancer or pigmentation.

As compounds with antagonism against MCH receptors, for example, WO 03/004027 (Patent Document 1) discloses a number of 4-phenylpiperidine derivatives. However, no compound with an imine skeleton is disclosed in the description of this patent.

WO 96/26196 (Patent Document 2) discloses benzylpiperidine derivatives as muscarine antagonists and compounds with an imine skeleton are also disclosed in the description thereof. However, no compound having both of a piperidine skeleton and an imine skeleton, i.e., the characteristic of the invention, is disclosed therein. Moreover, the description of this patent declares nothing about the antagonism against MCH receptors.

### Patent Document 1: WO 03/004027

### Patent Document 2: WO 96/26196

### DISCLOSURE OF THE INVENTION

### Problems that the Invention is to Solve

As the results of intensive studies on compounds with antagonism against MCH receptors, the present inventors have found out that a compound, in which two aryl groups are attached to a carbon atom of imine and piperidine is further attached via methylene to one of the aryl groups, is a novel compound that has never been reported in documents, has antagonism against an MCH receptor and is efficacious in preventing, treating or remedying various diseases in which the MCH receptor participates. The invention has been completed based on this finding.

Accordingly, the invention provides:

(1) a compound represented by the formula (I) or a pharmaceutically acceptable salt thereof:

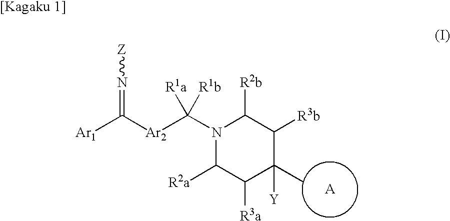

wherein,

R^{1a} and R^{1b} each independently represents a hydrogen atom or a C₁₋₆ alkyl group optionally having a substituent;

R^{2a} and R^{2b} each independently represents a hydrogen atom or a C₁₋₆ alkyl group optionally having a substituent, or R^{2a} and R^{2b} form together —C(R⁴)₂—C(R⁵)₂—;

R^{3a} and R^{3b} each independently represents a hydrogen atom or a C₁₋₆ alkyl group optionally having a substituent, or R^{3a} and R^{3b} form together —C(R⁶)₂—C(R⁷)₂—;

R⁴, R⁵, R⁶ and R⁷ each independently represents a hydrogen atom or a C₁₋₆ alkyl group optionally having a substituent;

Y represents a hydrogen atom or a hydroxy group;

Z represents —OR⁸, —N(R^{9a})(R^{9b}), —N(R¹⁰)—COO(R¹¹), —N(R¹²)—CO(R¹³), —C(R^{14a})(R^{14b})(R^{14c}), —O—SO₂R¹⁵ or —SO₂R¹⁶;

R⁸ represents a hydrogen atom, a C₁₋₆ alkyl group optionally having a substituent or a C₃₋₈ cycloalkyl group optionally having a substituent, wherein the C₁₋₆ alkyl group or C₃₋₈ cycloalkyl group is optionally substituted by a substituent selected from the group consisting of halogen, hydroxy, C₁₋₆ alkoxy, C₁₋₆ alkoxycarbonyl, C₁₋₆ alkylsulfonyl, (C₁₋₆ alkyl)amino, di(C₁₋₆ alkyl)amino, carbamoyl, (C₁₋₆ alkyl)carbamoyl, di(C₁₋₆ alkyl)carbamoyl and cyano groups;

R^{9a}, R^{9b}, R¹⁰, R¹¹, R¹², R¹³, R^{14a}, R^{14b} and R^{14c} each independently represents a hydrogen atom or a C₁₋₆ alkyl group optionally having a substituent;

R¹⁵ and R¹⁶ each independently represents a C₁₋₆ alkyl group or a phenyl group optionally substituted by a C₁₋₆ alkyl group;

Ar₁ represents an aromatic carbon ring group optionally substituted by a substituent selected from the group α, or a nitrogen-containing aromatic heterocyclic group optionally substituted by a substituent selected from the group α;

Ar₂ represents a group formed by removing two hydrogen atoms from an aromatic carbon ring group or an aromatic heterocyclic group wherein the aromatic carbon ring group or aromatic heterocyclic group is optionally substituted by a substituent selected from the group α; and

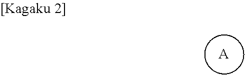

(hereinafter referred to the ring group A) represents

1) an aromatic carbon ring group or a partially unsaturated carbon ring group, or

2) an aromatic heterocyclic group or a partially unsaturated heterocyclic group, wherein the group is optionally substituted by a substituent selected from the group consisting of halogen, C₁₋₆ alkyl, C₁₋₆ haloalkyl, C₁₋₆ alkyloxy, C₁₋₆ alkylcarbonylamino, C₁₋₆ alkylcarbonyl-C₁₋₆ alkylamino, cyano and oxo groups.

Substituents constructing the group α:

halogen, cyano, hydroxy, amino, mono(C₁₋₆ alkyl)amino, di(C₁₋₆ alkyl)amino, C₁₋₆ alkyl, C₁₋₆ haloalkyl, C₁₋₆ alkyloxy, C₁₋₆ haloalkyloxy, C₁₋₆ alkyloxy-C₁₋₆ alkyl, C₁₋₆ alkyloxycarbonyl, C₁₋₆ alkyloxycarbonylamino, C₁₋₆ alkyloxycarbonyl(C₁₋₆ alkyl)amino, C₁₋₆ alkylcarbonyl, C₁₋₆ alkylcarbonyloxy, C₁₋₆ alkylcarbonylamino, C₁₋₆ alkylcarbonyl(C₁₋₆ alkyl)amino, carbamoyl, mono(C₁₋₆ alkyl)carbamoyl, di(C₁₋₆ alkyl)carbamoyl, carbamoylamino, mono(C₁₋₆ alkyl)carbamoylamino, di(C₁₋₆ alkyl)carbamoylamino, mono(C₁₋₆ alkyl)carbamoyl(Cᵢ-6 alkyl)amino, di(C₁₋₆ alkyl)carbamoyl(C₁₋₆ alkylamino, carbamoyloxy, mono(C₁₋₆ alkyl)carbamoyloxy, di(C₁₋₆ alkyl)carbamoyloxy, C₁₋₆ alkylsulfonyl, C₁₋₆ alkylsulfonylamino, C₁₋₆ alkylsulfonyl(C₁₋₆ alkyl)amino, sulfamoyl, mono(C₁₋₆alkyl)sulfamoyl, di(C₁₋₆ alkyl)sulfamoyl, sulfamoylamino, mono(C₁₋₆ alkyl)sulfamoylamino, di(C₁₋₆ alkyl)sulfamoylamino, mono(C₁₋₆ alkyl)sulfamoyl(C₁₋₆ alkyl)amino and di(C₁₋₆ alkyl)sulfamoyl(C₁₋₆ alkyl)amino groups.

Further, the invention provides:

(2) a melanin-concentrating hormone receptor antagonist which comprises a compound as described in (1) or a pharmaceutically acceptable salt thereof as the active ingredient;

(3) a medicinal composition which contains a pharmaceutically acceptable additive and a compound as described in (1) or a pharmaceutically acceptable salt thereof; and

(4) a preventive or remedy for obesity, diabetes, fatty liver, bulimia, depression or anxiety which comprises a compound as described in (1) or a pharmaceutically acceptable salt thereof as the active ingredient.

Furthermore, the invention provides a compound represented by the formula (Ia).

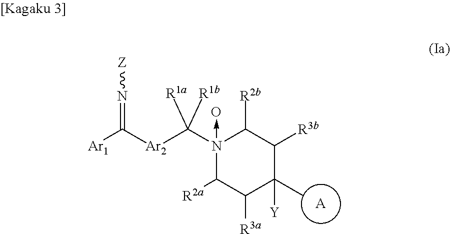

wherein each symbol has the same meaning as described above.

The compound represented by the formula (Ia) can be easily prepared by oxidizing a compound represented by the formula (I).

Now, the invention will be described in greater detail.

“Lower” as used herein means a group to which this term is attached has not more than 6 (more preferably not more than 4) carbon atoms.

Examples of the substituents in “C₁₋₆ alkyl group optionally having a substituent” in R^{1a}, R^{1b}, R^{2a}, R^{2b}, R^{3a}, R^{3b}, R⁴, R⁵, R⁶ and R⁷ include the substituents selected from the group consisting of the group α and R^{1a}, R^{1b}, R^{2a}, R^{2b}, R^{3a}, R^{3b}, R⁴, R⁵, R⁶ and R⁷ may be substituted by one or more of these substituents.

Substituents constructing the group α:

halogen, cyano, hydroxy, amino, mono(C₁₋₆ alkyl)amino, di(C₁₋₆ alkyl)amino, C₁₋₆ alkyl, C₁₋₆ haloalkyl, C₁₋₆ alkyloxy, C₁₋₆ haloalkyloxy, C₁₋₆ alkyloxy-C₁₋₆ alkyl, C₁₋₆ alkyloxycarbonyl, C₁₋₆ alkyloxycarbonylamino, C₁₋₆ alkyloxycarbonyl(C₁₋₆ alkyl)amino, C₁₋₆ alkylcarbonyl, C₁₋₆ alkylcarbonyloxy, C₁₋₆ alkylcarbonylamino, C₁₋₆ alkylcarbonyl(C₁₋₆ alkyl)amino, carbamoyl, mono(C₁₋₆alkyl)carbamoyl, di(C₁₋₆ alkyl)carbamoyl, carbamoylamino, mono(C₁₋₆ alkyl)carbamoylamino, di(C₁₋₆ alkyl)carbamoylamino, mono(C₁₋₆alkyl)carbamoyl(C₁₋₆ alkyl)amino, di(C₁₋₆ alkyl)carbamoyl(C₁₋₆ alkyl)amino, carbamoyloxy, mono(C₁₋₆ alkyl)carbamoyloxy, di(C₁₋₆ alkyl)carbamoyloxy, C₁₋₆ alkylsulfonyl, C₁₋₆ alkylsulfonylamino, C₁₋₆ alkylsulfonyl(C₁₋₆ alkyl)amino, sulfamoyl, mono(C₁₋₆ alkyl)sulfamoyl, di(C₁₋₆ alkyl)sulfamoyl, sulfamoylamino, mono(C₁₋₆ alkyl)sulfamoylamino, di(C₁₋₆ alkyl)sulfamoylamino, mono(C₁₋₆ alkyl)sulfamoyl(C₁₋₆ alkyl)amino and di(C₁₋₆ alkyl)sulfamoyl(C₁₋₆ alkyl)amino groups.

In defining the above substituents, “halogen” includes fluoro, chloro, bromo and iodo.

“C₁₋₆ alkyl group” means a linear alkyl group having 1 to 6 carbon atoms or a branched alkyl group having 3 to 6 carbon atoms. Specific examples thereof include methyl, ethyl, n-propyl, isopropyl, n-butyl, isobutyl, sec-butyl, tert-butyl, n-pentyl, isopentyl, neopentyl, tert-amyl, 2-propyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl groups and the like.

“C₃₋₈ cycloalkyl group” means a cycloalkyl group having 3 to 8 carbon atoms. Examples thereof include cyclopropyl, cyclobutyl, cyclopentyl, cyclohexyl, cycloheptyl and cyclooctyl groups.

“C₁₋₆ haloalkyl group” means a C₁₋₆ alkyl group in which all or some of hydrogen atoms are substituted by halogen atom(s). Examples thereof include fluoromethyl, difluoromethyl, trifluoromethyl, 2-fluoroethyl and 1,2-difluoroethyl groups and the like.

“C₁₋₆ alkyloxy group” means a group in which a C₁₋₆ alkyl group is attached to an oxygen atom. Specific examples thereof include methoxy, ethoxy, n-propyloxy, isopropyloxy, n-butoxy, isobutoxy, tert-butoxy and n-pentyloxy groups and the like.

“C₁₋₆ haloalkyloxy group” means a group in which a C₁₋₆ haloalkyl group is attached to an oxygen atom. Specific examples thereof include fluoromethoxy, chloromethoxy, difluoromethoxy, dichloromethoxy, trifluoromethoxy, trichloromethoxy, 2-fluoroethoxy and 1,2-difluoroethoxy groups and the like.

“Monohydroxy(C₁₋₆ alkyl)oxy group” means a group in which one hydrogen atom of a C₁₋₆ alkyloxy group is substituted by hydroxy. Specific examples thereof include a 2-hydroxyethoxy group and the like.

“Dihydroxy(C₁₋₆ alkyl)oxy group” means a group in which two hydrogen atoms of a C₁₋₆ alkyloxy group are substituted by hydroxy. Specific examples thereof include a 1,2-dihydroxyethoxy group and the like.

“Mono(C₁₋₆ alkyl)amino group” means a group in which one hydrogen atom of an amino group (—NH₂) is substituted by a C₁₋₆ alkyl group. Specific examples thereof include methylamino, ethylamino, n-propylamino, isopropylamino, n-butylamino, sec-butylamino and tert-butylamino groups and the like.

“Di(C₁₋₆ alkyl)amino group” means a group in which two hydrogen atoms of an amino group (—NH₂) are substituted by C₁₋₆ alkyl groups. Specific examples thereof include dimethylamino, diethylamino, ethylmethylamino, di(n-propyl)amino, methyl(n-propyl)amino and diisopropylamino groups and the like.

“C₁₋₆ alkyloxy-C₁₋₆ alkyl group” means a C₁₋₆ alkyl group substituted by a C₁₋₆ alkyloxy group. Specific examples thereof include methoxymethyl, ethoxymethyl, n-propyloxymethyl, isopropyloxymethyl, 1-methoxyethyl and 2-methoxyethyl groups and the like.

“C₁₋₆ alkyloxycarbonyl group” means a group in which a C₁₋₆ alkyloxy group is attached to a carbonyl group (—CO—) and includes alkyloxycarbonyl groups having 1 to 6 carbon atoms. Specific examples thereof include methoxycarbonyl, ethoxycarbonyyl, n-propyloxycarbonyl, isopropyloxycarbonyl, n-butoxycarbonyl, isobutoxycarbonyl, tert-butoxycarbonyl and n-pentyloxycarbonyl groups and the like.

“C₁₋₆ alkyloxycarbonylamino group” means a group in which one hydrogen atom of an amino group is substituted by a C₁₋₆ alkyloxycarbonyl group and includes alkyloxycarbonylamino groups having 1 to 6 carbon atoms. Specific examples thereof include methoxycarbonylamino, ethoxycarbonylamino, n-propyloxycarbonylamino, isopropyloxycarbonylamino, n-butoxycarbonylamino, isobutoxycarbonylamino, tert-butoxycarbonylamino and n-pentyloxycarbonylamino groups and the like.

“C₁₋₆ alkyloxycarbonyl(C₁₋₆ alkyl)amino group” means a group in which a hydrogen atom on the nitrogen atom of a mono(C₁₋₆)alkylamino group is substituted by a C₁₋₆ alkyloxycarbonyl group. Specific examples thereof include methoxycarbonyl(methyl)amino, ethoxycarbonyl(methyl)amino and n-propyloxycarbonyl(methyl)amino groups and the like.

“C₁₋₆ alkylcarbonyl group” means a group in which a C₁₋₆ alkyl group is attached to a carbonyl group and includes alkyl carbonyl groups having 1 to 6 carbon atoms. Specific examples thereof include acetyl, propionyl, butyryl, isobutyryl, valeryl, isovaleryl and pivaloyl groups and the like.

“C₁₋₆ alkylcarbonyloxy group” means a group in which a C₁₋₆ alkylcarbonyl is attached to an oxygen atom. Specific examples thereof include acetoxy, propionyloxy, valeryloxy, isovaleryloxy and pivaloyloxy groups and the like.

“C₁₋₆ alkylcarbonylamino group” means a group in which one hydrogen atom of an amino group is substituted by a C₁₋₆ alkylcarbonyl group. Specific examples thereof include acetylamino, propionylamino, isobutyrylamino, valerylamino, isovalerylamino and pivaloylamino groups and the like.

“C₁₋₆ alkylcarbonyl(C₁₋₆ alkyl)amino group” means a group in which a hydrogen atom on the nitrogen atom of a mono(C₁₋₆ alkyl)amino group is substituted by a C₁₋₆ alkylcarbonyl group. Specific examples thereof include methylcarbonyl(methyl)amino, ethylcarbonyl(methyl)amino and n-propylcarbonyl(methyl)amino groups and the like.

“Mono(C₁₋₆ alkyl)carbamoyl group” means a group in which one hydrogen atom of a carbamoyl group (—CONH₂) is substituted by a C₁₋₆ alkyl group. Specific examples thereof include methylcarbamoyl, ethylcarbamoyl, n-propylcarbamoyl, isopropylcarbamoyl, n-butylcarbamoyl, sec-butylcarbamoyl and tert-butylcarbamoyl groups and the like.

“Di(C₁₋₆ alkyl)carbamoyl group” means a group in which two hydrogen atoms of a carbamoyl group are substituted by C₁₋₆ alkyl groups. Specific examples thereof include dimethylcarbamoyl, diethylcarbamoyl, ethylmethylcarbamoyl, di(n-propyl)carbamoyl, methyl(n-propyl)carbamoyl and diisopropylcarbamoyl groups and the like.

“Mono(C₁₋₆ alkyl)carbamoylamino group” means a group in which one hydrogen atom of an amino group is substituted by a mono(C₁₋₆ alkyl)carbamoyl group. Specific examples thereof include methylcarbamoylamino, ethylcarbamoylamino, n-propylcarbamoylamino, isopropylcarbamoylamino, n-butylcarbamoylamino, sec-butylcarbamoylamino and tert-butylcarbamoylamino groups and the like.

“Di(C₁₋₆ alkyl)carbamoylamino group” means a group in which one hydrogen atom of an amino group is substituted by a di(C₁₋₆ alkyl)carbamoyl group. Specific examples thereof include dimethylcarbamoylamino, diethylcarbamoylamino, di(n-propyl)carbamoylamino, diisopropylcarbamoylamino, di(n-butyl)carbamoylamino, di(sec-butyl)carbamoylamino and di(tert-butyl)carbamoylamino groups and the like.

“Mono(C₁₋₆ alkyl)carbamoyl(C₁₋₆ alkyl)amino group” means a group in which a hydrogen atom on the nitrogen atom of a mono(C₁₋₆ alkyl)amino group is substituted by a mono(C₁₋₆ alkyl)carbamoyl group. Specific examples thereof include monomethylcarbamoyl(methyl)amino, monoethylcarbamoyl(methyl)amino and [mono(n-propyl)carbamoyl](methyl)amino groups and the like.

“Di(C₁₋₆ alkyl)carbamoyl(C₁₋₆ alkyl)amino group” means a group in which a hydrogen atom on the nitrogen atom of a mono(C₁₋₆ alkyl)amino group is substituted by a di(C₁₋₆ alkyl)carbamoyl group. Specific examples thereof include dimethylcarbamoyl(methyl)amino, diethylcarbamoyl(methyl)amino and [di(n-propyl)carbamoyl](methyl)amino groups and the like.

“Mono(C₁₋₆ alkyl)carbamoyloxy group” means a group in which a mono(C₁₋₆ alkyl)carbamoyl group is attached to an oxygen atom. Specific examples thereof include methylcarbamoyloxy, ethylcarbamoyloxy, n-propylcarbamoyloxy, isopropylcarbamoyloxy, n-butylcarbamoyloxy, sec-butylcarbamoyloxy and tert-butylcarbamoyloxy groups and the like.

“Di(C₁₋₆ alkyl)carbamoyloxy group” means a group in which a di(C₁₋₆ alkyl)carbamoyl group is attached to an oxygen atom. Specific examples thereof include dimethylcarbamoyloxy, diethylcarbamoyloxy, ethylmethylcarbamoyloxy, di(n-propyl)carbamoyloxy, methyl(n-propyl)carbamoyloxy and diisopropylcarbamoyloxy groups and the like.

“C₁₋₆ alkylsulfonyl” group means a group in which a C₁₋₆ alkyl group is attached to a sulfonyl (—SO₂—) group. Specific examples thereof include methanesulfonyl, ethanesulfonyl, n-propylsulfonyl, isopropanesulfonyl, n-butanesulfonyl, sec-butanesulfonyl and tert-butanesulfonyl groups and the like.

“C₁₋₆alkylsulfonylamino group” means a group in which one hydrogen atom of an amino group is substituted by a C₁₋₆ alkylsulfonyl group. Specific examples thereof include methanesulfonylamino, ethanesulfonylamino, n-propanesulfonylamino, isopropanesulfonylamino, n-butanesulfonylamino, sec-butanesulfonylatnino and tert-butanesulfonylamino groups and the like.

“C₁₋₆ alkylsulfonyl(C₁₋₆ alkyl)amino group” means a group in which a hydrogen atom on the nitrogen atom of a mono(C₁₋₆ alkyl)amino group is substituted by a C₁₋₆ alkylsulfonyl group. Specific examples thereof include methanesulfonyl(methyl)amino, ethanesulfonyl(methyl)amino, n-propanesulfonyl(methyl)amino and isopropanesulfonyl(methyl)amino groups and the like.

“Mono(C₁₋₆ alkyl)sulfamoyl group” means a group in which a hydrogen atom of a sulfamoyl (—SO₂NH₂) group is substituted by a C₁₋₆ alkyl group. Specific examples thereof include monomethylsulfamoyl, monoethylsulfamoyl, mono(n-propyl)sulfamoyl, mono isopropylsulfamoyl, mono(n-butyl)sulfamoyl, mono(sec-butyl)sulfamoyl and mono(tert-butyl) sulfamoyl groups and the like.

“Di(C₁₋₆ alkyl)sulfamoyl group” means a group in which two hydrogen atoms of a sulfamoyl group are substituted by C₁₋₆ alkyl groups. Specific examples thereof include dimethylsulfamoyl, diethylsulfamoyl, di(n-propyl)sulfamoyl, diisopropylsulfamoyl, di(n-butyl)sulfamoyl, di(sec-butyl)sulfamoyl and di(tert-butyl)sulfamoyl groups and the like.

“Mono(C₁₋₆ alkyl)sulfamoylamino group” means a group in which one hydrogen atom of an amino group is substituted by a mono(C₁₋₆ alkyl)sulfamoyl group. Specific examples thereof include (monomethylsulfamoyl)amino, (monoethylsulfamoyl)amino, [mono(n-propyl)sulfamoyl]amino, (monoisopropylsulfamoyl)amino, [mono(n-butyl)sulfamoyl]amino, [mono(sec-butyl)sulfamoyl]amino and [mono(tert-butyl)sulfamoyl]amino groups and the like.

“Di(C₁₋₆ alkyl)sulfamoylamino group” means a group in which one hydrogen atom of an amino group is substituted by a di(C₁₋₆ alkyl)sulfamoyl group. Specific examples thereof include (dimethylsulfamoyl)amino, (diethylsulfamoyl)amino, (ethylmethylsulfamoyl)amino, [di(n-propyl)sulfamoyl]amino, [methyl(n-propyl)sulfamoyl]amino and (diisopropylsulfamoyl)amino groups and the like.

“Mono(C₁₋₆ alkyl)sulfamoyl(C₁₋₆ alkyl)amino group” means a group in which a hydrogen atom on the nitrogen atom of a mono(C₁₋₆ alkyl)amino group is substituted by a mono(C₁₋₆ alkyl)sulfamoyl group. Specific examples thereof include monomethylsulfamoyl(methyl)amino, monoethylsulfamoyl(methyl)amino and [mono(n-propyl)sulfamoyl](methyl)amino groups and the like.

“Di(C₁₋₆ alkyl)sulfamoyl(C₁₋₆ alkyl)amino group” means a group in which a hydrogen atom on the nitrogen atom of a mono(C₁₋₆ alkyl)amino group is substituted by a di(C₃₋₆ alkyl)sulfamoyl group. Specific examples thereof include dimethylsulfamoyl(methyl)amino, diethylsulfamoyl(methyl)amino and [di(n-propyl)sulfamoyl](methyl)amino groups and the like.

Examples of “aromatic carbon ring” include phenyl, naphthyl and the like.

“Partially unsaturated carbon ring” means a “partially unsaturated” or “unsaturated” ring or ring system having one or more double bonds. It may be either a monocyclic or bicyclic ring. Examples thereof include indane, indene, dehydronaphthalene and the like.

“Aromatic heterocycle” means a 5- or 6-membered monocyclic heteroaryl containing 1 or more (preferably 1 to 3) hetero atoms, which may be the same or different and are selected from the group consisting of oxygen, nitrogen and sulfur atoms, or a fused ring type heteroaryl in which the preceding monocyclic heteroaryl is fused with the above-described aryl, or the preceding monocyclic heteroaryls, which may be either the same or different, are fused together. Specific examples thereof include pyrrolyl, furyl, thienyl, imidazolyl, pyrazolyl, thiazolyl, isothiazolyl, oxazolyl, isooxazolyl, triazolyl, tetrazolyl, oxadiazolyl, 1,2,3-thidiazolyl, 1,2,4-thidiazolyl, 1,3,4-thidiazolyl, pyridyl, pyrazinyl, pyrimidinyl, pyridazinyl, 1,2,4-triazinyl, 1,3,5-triazinyl, indolyl, benzofuranyl, benzothienyl, benzoimidazolyl, benzopyrazolyl, benzoxazolyl, benzoisoxazolyl, benzothiazolyl, benzoisothiazolyl, indazolyl, purinyl, quinolyl, isoquinolyl, phthaladinyl, naphthylidinyl, quinoxalinyl, quinazolinyl, cinnolinyl, puteridinyl, pyrido[3,2-b]pyridyl and the like.

“Partially unsaturated heterocycle” means a heterocycle which is partially unsaturated and specific examples thereof include 2,3-dihydro-1H-pyrrolo[2,3-b]pyridine, 1,2-dihydro-2H-pyridine, 3,6-dihydro-2H-pyridine, 1,2-dihydropyrimidine, 1,2-dihydropyrazine, indoline and the like.

“Pharmaceutically acceptable salt” of the derivative represented by the formula (I) means a pharmaceutically acceptable and common salt. Examples thereof include acid addition salts at the amine moiety of the compound represented by the formula (I), acid addition salts at the nitrogen-containing heterocycle thereof, base addition salts at an acidic substituent of the compound represented by the formula (I) in the case of having the same, and the like.

Examples of the acid addition salt include inorganic acid salts such as hydrochlorides, sulfates, nitrates, phosphates, perchlorates and so on; organic acid salts such as maleates, fumarates, tartrates, citrates, ascorbates, trifluoroacetates and so on; and sulfonates such as methanesulfonates, isothiocyanates, benzenesulfonates, p-toluenesulfonates and so on.

Examples of the base addition salt include alkali metal salts such as sodium salts, potassium salts and so on; alkaline earth metal salts such as calcium salts, magnesium salts and so on; ammonium salts; and organic amine salts such as trimethylamine salts, triethylamine salts, dicyclohexylamine salts, ethanolamine salts, diethanolamine salts, triethanolamine salts, procaine salts, N,N′-dibenzylethylenediamine salts and so on.

To further disclose the derivatives according to the invention, the individual symbols used in the formula (I) and so on will be described in greater detail by citing preferred examples thereof.

R^{1a} and R^{1b} each independently represents a hydrogen atom or a C₁₋₆ alkyl group optionally having a substituent.

As examples of the substituent in the C₁₋₆ alkyl group optionally having a substituent, halogen such as fluoro, chloro and so on may be cited.

Specific examples of R^{1a} and R^{1b} include a hydrogen atom, methyl, ethyl n-propyl and the like and hydrogen atom is particularly recommended.

R^{2a} and R^{2b} each independently represents a hydrogen atom or a C₁₋₆ alkyl group optionally having a substituent, or R^{2a} and R^{2b} form together —C(R⁴)₂—C(R⁵)₂—.

R⁴ and R⁵ each independently represents a hydrogen atom or a C₁₋₆ alkyl group optionally having a substituent.

As examples of the substituent in the C₁₋₆ alkyl group optionally having a substituent, halogen such as fluoro, chloro and so on may be cited.

Specific examples of R^{2a} and R^{2b} include a hydrogen atom, methyl, ethyl, n-propyl, isopropyl, n-butyl, tert-butyl and so on; and groups consisting of R^{2a} and R^{2b} bonded to each other such as —CH₂CH₂—, —CH₂—CH(CH₃)— and —CH(CH₃)—CH₂—. Among them, a hydrogen atom, methyl and —CH₂CH₂— consisting of R^{2a} and R^{2b} bonded to each other are recommended as preferable ones.

R^{3a} and R^{3b} each independently represents a hydrogen atom or a C₁₋₆ alkyl group optionally having a substituent. Alternatively, R^{3a} and R^{3b} form together —C(R⁶)₂—C(R⁷)₂—.

R⁶ and R⁷ each independently represents a hydrogen atom or a C₁₋₆ alkyl group optionally having a substituent.

As examples of the substituent in the C₁₋₆ alkyl group optionally having a substituent, halogen such as fluoro, chloro and so on may be cited.

Specific examples of R^{3a} and R^{3b} include a hydrogen atom, methyl, ethyl, n-propyl, isopropyl, n-butyl, tert-butyl and so on; and groups consisting of R^{3a} and R^{3b} bonded to each other such as —CH₂CH₂—, —CH₂—CH(CH₃)— and —CH(CH₃)—CH₂—. It is preferable and recommended that R^{3a} and R^{3b} are both hydrogen atoms.

Y represents a hydrogen atom or a hydroxy group and a hydrogen atom is preferred.

Z represents —OR⁸, —N(R^{9a})(R^{9b})₂, —N(R¹⁰)—COO(R¹¹), —N(R¹²)—CO(R¹³), —C(R^{14a})(R^{14b})(R^{14c}), —O—SO₂R¹⁵ or —SO₂R¹⁶.

R⁸ represents a hydrogen atom, a C₁₋₆ alkyl group optionally having a substituent or a C₃₋₄ cycloalkyl group optionally having a substituent, wherein the C₁₋₆ alkyl group or C₃₋₄ cycloalkyl group is optionally substituted by a substituent selected from the group consisting of halogen, hydroxy, C₁₋₆ alkoxy, C₁₋₆ alkoxycarbonyl, C₁₋₆ alkylsulfonyl, alkyl)amino, di(C₁₋₆ alkyl)amino, carbamoyl, (C₁₋₆ alkyl)carbamoyl, di(C₁₋₆ alkyl)carbamoyl and cyano groups.

R^{9a}, R^{9b}, R¹⁰, R¹¹, R¹², R¹³, R^{14a}, R^{14b} and R^{14c} each independently represents a hydrogen atom or a C₁₋₆ alkyl group optionally having a substituent.

In the case where R^{9a}, R^{9b}, R¹⁰, R¹¹, R¹², R¹³, R^{14a}, R^{14b} or R^{14c} is “a C₁₋₆ alkyl group optionally having a substituent”, preferable examples of the substituent include halogen such as fluoro and chloro.

R¹⁵ and R¹⁶ each independently represents a C₁₋₆ alkyl group or a phenyl group optionally substituted by a C₁₋₆ alkyl group.

As Z,

1) specific examples of —OR⁸ include hydroxy, methoxy, ethoxy, n-propyloxy, isopropyloxy, fluoromethoxy, difluoromethoxy, trifluoromethoxy, 2-fluoroethoxy, 2-chloroethoxy, 2,2-difluoroethoxy, 2-methoxyethoxy, 2-hydroxyethoxy, 2-hydroxy-2-methylpropyloxy, methoxycarbonylmethoxy, carbamoylmethoxy, methylcarbamoylmethoxy, dimethylcarbamoylmethoxy, 2-dimethylaminoethoxy, cyanomethyloxy, cyanoethyloxy and cyclopropyloxy;

2) specific examples of —N(R^{9a})(R^{9b}) include amino, dimethylamino and diethylamino;

3) specific examples of —N(R¹⁰)—COO(R¹¹) include methoxycarbonylamino and ethoxycarbonylamino;

4) specific examples of —N(R¹²)—CO(R¹³) include methylcarboxamino and ethylcarboxamino;

5) specific examples of —C(R^{14a})(R^{14b})(R^{14c}) include methyl, ethyl, n-butyl, isobutyl, t-butyl and difluoromethyl;

6) specific examples of —O—SO₂R¹⁵ include methylsulfonyloxy, ethylsulfonyloxy, p-toluenesulfonyloxy and benzenesulfonyloxy; and

7) specific examples of —SO₂R¹⁶ include methylsulfonyl and ethylsulfonyl.

Among all, —OR⁸ or —O—SO₂R¹⁵ (in particular, —OR⁸) is preferable as Z. Recommended examples thereof include hydroxy, methoxy, ethoxy, n-propyloxy, isopropyloxy, cyclopropyloxy, 2-fluoroethoxy, 2,2-difluoroethoxy, 2,2,2-trifluoroethoxy, 2-hydroxyethoxy, 2-hydroxypropyloxy, dimethylaminoethoxy, dimethylcarbamoylmethoxy, difluoromethoxy, 2-hydroxy-2-methylpropyloxy, cyanomethyloxy, methylsulfonyloxy and so on. Particularly recommended are ethoxy, cyclopropyloxy, 2,2-difluoroethoxy, 2-hydroxy-2-methylpropyloxy, cyanomethyloxy and methylsulfonyloxy.

Ar₁ represents an aromatic carbon ring group optionally substituted by a substituent selected from the group α, or a nitrogen-containing aromatic heterocyclic group optionally substituted by a substituent selected from the group α.

As the aromatic carbon ring group or nitrogen-containing aromatic heterocyclic group represented by Ar₁, a 6-membered aromatic carbon ring group or a 6-membered nitrogen-containing aromatic heterocyclic group may be cited. Examples of the aromatic carbon ring in the 6-membered aromatic carbon ring group include a benzene ring, while examples of the nitrogen-containing aromatic heterocycle in the 6-membered nitrogen-containing aromatic heterocyclic group include a pyridine ring, a pyrazine ring, a pyrimidine ring and a pyradazine ring.

Preferable examples of the substituent selected from the group α in Ar₁ include halogen such as fluoro, chloro or bromo; and C₁₋₆ alkyl such as methyl, ethyl, n-propyl or isopropyl which may have one to four (preferably one or two) substituents.

Specific examples of Ar₁ include 6-membered aromatic carbon ring groups such as phenyl, 4-fluorophenyl and 3,4-difluorophenyl; and 6-membered nitrogen-containing aromatic heterocyclic groups such as pyridyl, 5-fluoropyridin-2-yl, 5-chloropyridin-2-yl and 6-chloropyridin-3-yl groups. As particularly preferable examples thereof, 6-membered aromatic carbon ring groups and 6-membered nitrogen-containing aromatic heterocyclic groups substituted by fluorine or chlorine atoms may be cited. In particular, 3,4-difluorophenyl and 5-chloropyridin-2-yl groups are recommended.

Ar₂ represents a group formed by removing two hydrogen atoms from an aromatic carbon ring group or an aromatic heterocyclic group wherein the aromatic carbon ring group or aromatic heterocyclic group is optionally substituted by a substituent selected from the group α.

As the aromatic carbon ring group represented by Ar₂, a 6-membered aromatic carbon ring group may be cited and examples thereof include or a benzene ring. As the aromatic heterocyclic group represented by Ar₂, a nitrogen-containing aromatic heterocyclic group, in particular, a 6-membered nitrogen-containing aromatic heterocyclic group may be cited and examples thereof include a pyridine ring, a pyrazine ring, a pyrimidine ring and a pyridazine ring.

Preferable examples of the substituent selected from the group a in Ar₂ include fluoro, chloro, methyl, ethyl, n-propyl, isopropyl, chloromethyl, fluoromethyl, methoxy, ethoxy, methylcarbonyl, methaneslufonyl and so on.

The substituent selected from the group α in Ar₁ and the substituent selected from the group a in Ar₂ may be either the same or different.

Preferable examples of the 6-membered aromatic carbon ring group include a 1,4-phenylenediyl group and so on; while preferable examples of the 6-membered nitrogen-containing aromatic heterocyclic group include a pyridine-2,5-diyl group, a pyrimidin-2,5-diyl group and so on.

As Ar₂, 1,4-phenylenediyl, 3-methanesulfonlyphenylene-1,4-diyl, pyridin-2,5-diyl, pyrimidine-2,5-diyl and so on are preferably recommended.

The ring group A represents:

1) an aromatic carbon ring group or a partially unsaturated carbon ring group, or

2) an aromatic heterocyclic group or a partially unsaturated heterocyclic group, wherein the group is optionally substituted by a substituent selected from the group consisting of halogen, C₁₋₆ alkyl, C₁₋₆ haloalkyl, C₁₋₆ alkyloxy, C₁₋₆ alkylcarbonylamino, C₁₋₆ alkylcarbonyl-C₁₋₆ alkylamino, cyano and oxo groups.

The aromatic carbon ring or partially unsaturated carbon ring in the ring group A means a monocyclic or bicyclic group. Specific examples thereof include a benzene ring, naphthalene, indane, dehydronaphthalene and so on.

The aromatic heterocyclic group or a partially unsaturated heterocyclic group in the ring group A means a monocyclic or bicyclic group. Specific examples thereof include pyridine, pyridazine, pyrimidine, pyrazine, imidazo[1,2-a]pyridine, imidazo[1,2-a]pyrimidine, imidazo[1,2-a]pyrazine, imidazo[1,2-b]pyridazine, 1H-pyrrolo[2,3-c]pyridine, 2,3-dihydro-1,3-benzoxazole, [1,2,4]triazolo-[4,3-a]pyridine, [1,2,4]triazolo-[1,5-a]pyridine, pyrazolo[1,5-b]pyridazine, 2,3-dihydro-1H-pyrrolo[2,3-b]pyridine, 1,2-dihydropyridine, 1,2-dihydropyrimidine, 1,2-dihydropyrazine, indole, indoline, 1H-pyrazole, isothiazole ring and so on.

It is preferable that the optional substituent of the ring group A is nil or halogen, C₁₋₆ alkylcarbonylamino, C₁₋₆ alkyl or oxo. Specific examples thereof include halogen such as fluoro, chloro and so on; C₁₋₆ alkylcarbonylamino groups such as methylcarbonylamino, ethylcarbonylamino, isopropylcarbonylamino and so on; an oxy group; and C₁₋₆ alkyl groups such as methyl, ethyl, n-propyl, isopropyl and so on. It is still preferred that the optional substituent is either nil or fluoro, methylcarbonylamino, methyl, etc.

Specific examples of the ring group A include phenyl, 3-methylcarboxaminophenyl, pyridin-2-yl, pyridin-3-yl, pyridin-4-yl, 2-fluoropyridin-3-yl, 2-fluoropyridin-5-yl, 2-fluoropyridin-6-yl, 3-fluoropyridin-5-yl, 3-fluoropyridin-6-yl, 6-fluoropyridin-3-yl, 2-methylpyridin-5-yl, 2-ethoxypyridin-5-yl, 2-methylcarboxaminopyridin-4-yl, 2-methylcarboxaminopyridin-6-yl, 3-methylcarboxaminopyridin-5-yl, 5-methylcarboxaminopyridin-3-yl, 3-isopropylcarboxaminopyridin-5-yl, 3-cyanopyridin-5-yl, 1-oxidopyridin-3-yl, 1-oxidopyridin-4-yl, 1-methyl-1H-pyrazol-4-yl, 1-methyl-1,2-dehydropyridin-2-on-4-yl, 1-methyl-1,6-dehydropyridin-6-on-4-yl, 1-difluoromethyl-1,2-dehydropyridin-2-on-4-yl, N-methoxy-1,2-dehydropyridin-2-on-4-yl, 1,3-dihydro-2H-indol-2-on-6-yl, 1,3-dihydro-2H-indol-2-on-7-yl, 2-oxo-2,3-dihydro-1,3-benzoxazol-5-yl, imidazo[1,2-a]pyridin-6-yl, 2-methylimidazo[1,2-a]pyridin-6-yl, 2,3-dimethylimidazo[1,2-a]pyridin-6-yl, imidazo[1,2-a]pyridin-7-yl, imidazo[1,2-a]pyrazin-3-yl, imidazo[1,2-a]pyrimidin-3-yl, 1H-pyrrolo[2,3-c]pyridin-3-yl, 1-methyl-1H-pyrrolo[2,3-c]pyridin-3-yl, [1,2,4]triazolo[4,3-a]pyridin-7-yl, [1,2,4]triazolo[4,3-a]pyridin-6-yl, [1,2,4]triazolo[1,5-a]pyridin-7-yl and pyrazolo[1,5-b]pyridazin-3-yl groups and so on. Preferably recommended are 6-fluoropyridin-3-yl, 5-methylcarboxaminopyridin-3-yl, 1-oxidopyridin-3-yl, 1-oxidopyridin-4-yl, 1-methyl-1H-pyrazol-4-yl, 1-methyl-1,2-dehydropyridin-2-on-4-yl, 1-methyl-1,6-dehydropyridin-6-on-4-yl, 2-oxo-2,3-dihydro-1,3-benzoxazol-5-yl, imidazo[1,2-a]pyridin-7-yl, imidazo[1,2-a]pyrazin-3-yl, imidazo[1,2-a]pyrimidin-3-yl, 1-methyl-1H-pyrrolo[2,3-c]pyridin-3-yl, [1,2,4]triazolo[4,3-a]pyridin-7-yl, [1,2,4]triazolo[1,5-a]pyridin-7-yl and pyrazolo[1,5-b]pyridazin-3-yl groups and so on.

Preferred examples of the compounds according to the invention are as follows:
N-{5-[1-({6-[(E)-(3,4-difluorophenyl)(methoxyimino)methyl]pyridin-3-yl}methyl)piperidin-4-yl]pyridin-3-yl}acetamide,
N-{5-[1-({6-[(E)-[(2,2-difluoroethoxy)imino](3,4-difluorophenyl)methyl]pyridin-3-yl}methyl)piperidin-4-yl]pyridin-3-yl}acetamide,
(E)-(3,4-difluorophenyl)(5-{[4-(1-methyl-1H-pyrazol-4-yl)piperidin-1-yl]methyl}pyridin-2-yl)methanone O-ethyloxime,
(E)-(3,4-difluorophenyl){5-[(4-imidazo[1,2-a]pyridin-7-yl-piperidin-1-yl)methyl]pyridin-2-yl}methanone O-ethyloxime,
5-[1-({6-(E)-(3,4-difluorophenyl)(ethoxyimino)methyl]pyridin-3-yl}methyl)piperidin-4-yl]-1,3-benzoxazole-2(3H)-one,
(E)-(3,4-difluorophenyl)(5-{[4-(1-methyl-1H-pyrrolo[2,3-c]pyridin-3-yl)-piperidin-1-yl]methyl}pyridin-2-yl)methanone O-ethyloxime,
(E)-(3,4-difluorophenyl){5-[(4-imidazo[1,2-a]pyrazin-3-yl)-piperidin-1-yl]methyl}pyridin-2-yl}methanone O-ethyloxime,
(E)-(3,4-difluorophenyl)(5-{[4-(6-fluoropyridin-3-yl)piperidin-1-yl]methyl}pyridin-2-yl)methanone O-(2-hydroxy-2-methylpropyl)oxime,
(E)-(3,4-difluorophenyl){5-[(4-pyrazolo[1,5-b]pyridazin-3-ylpiperidin-1-yl)methyl]pyridin-2-yl}methasone O-(2-hydroxy-2-methylpropyl)oxime,
(E)-(3,4-difluorophenyl)(5-{[4-(6-fluoropyridin-3-yl)piperidin-1-yl]methyl}pyridin-2-yl)methanone O-[(methylsulfonyl)methyl]oxime,
(E)-(3,4-difluorophenyl) {5-[(4-[1,2,4]-triazolo[4,3-a]pyridin-7-ylpiperidin-1-yl)methyl]pyridin-2-yl}methanone O-cyclopropyloxime,
{[((1E)-(3,4-difluorophenyl) {5-[(4-[1,2,4-triazolo[1,5-a]pyridin-7-ylpipetidin-1-yl)methyl]pyridin-2-yl}methylene)amino]oxy}acetonitrile,
5-[1-(1-{6-[(E)-(3,4-difluorophenyl)(ethoxyimino)methyl]pyridin-3-yl}ethyl)piperidin-4-yl]-1-methylpyridin-2(1H)-one,
4-[1-({5-[(E)-(3,4-difluorophenyl)(ethoxyimino)methyl]pyridin-2-yl}methyl)piperidin-4-yl]-1-methylpyridin-2(1H)-one,
(E)-(3,4-difluorophenyl)(6-{[4-(1-oxidopyridin-3-yl)piperidin-1-yl]methyl}pyridin-3-yl)methanone O-ethyloxime,
(Z)-(3,4-difluorophenyl){4-[(4-imidazo[1,2-a]pyrimidin-3-ylpiperidin-1-yl)methyl]phenyl}methanone O-(2-hydroxy-2-methylpropyl)oxime,
({[(1Z)-(3,4-difluorophenyl)(4-{[4-(1-oxidopyridin-4-yl)piperidin-1-yl]methyl}phenyl)methylene]amino}oxy)acetonitrile,
5-(1-{4-[(Z)-(5-chloropyridin-2-yl)(ethoxyimino)methyl]benzyl}piperidin-4-yl]-1-methylpyridin-2(1H)-one,
(Z)-(5-chloropyridin-2-yl){4-[(4-[1,2,4]triazolo[4,3-a]pyridin-7-ylpiperidin-1-yl)methyl]phenyl}methanone O-ethyloxime,
(E)-(3,4-difluorophenyl)(5-{[4-(6-fluoropyridin-3-yl)piperidin-1-yl]methyl}pyrimidin-2-yl)methanone-O-cyclopropyloxime,
({[(1E)-(3,4-difluorophenyl)(5-{[4-(6-fluoropyridin-3-yl]piperidin-1-yl]methyl}pyridin-2-yl)methylene]amino}oxy)acetonitrile, and so on.

### Method of Producing Compounds Represented by the Formula (I)

The compounds represented by the formula (I) can be prepared by, for example, the following production methods, though the invention is not restricted thereto.

### Production Method 1-1

In the production method 1-1, a compound represented by the formula (I) is obtained by reacting a compound represented by the formula (II) with a compound represented by the formula (III).

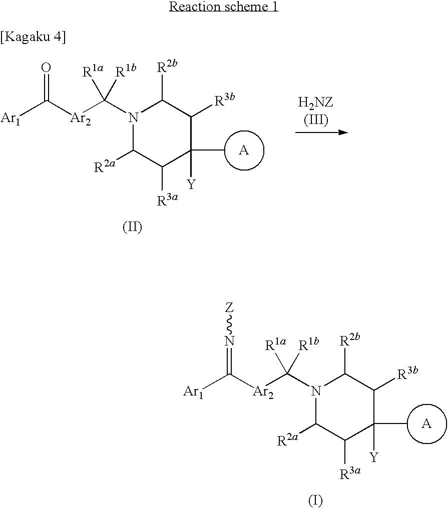

wherein each symbol is as defined above.

Namely, the compound represented by the formula (I) can be obtained by using the compound represented by the formula (II) and the compound represented by the formula (III) and conducting oximation or hydrazonation in accordance with a publicly known method.

The compound represented by the formula (III) may be used in an amount of, for example, 1.0 to 5.0 mol per mol of the compound represented by the formula (II) and preferably recommended is 1.0 to 1.5 mol.

1) Reaction Conditions for Oximation

Examples of the reaction solvent include lower alcohols such as methanol, ethanol, n-butanol and isopropyl alcohol, pyridine and so on.

The reaction temperature is, for example, 0 to 100° C. and preferably recommended is 10 to 30° C. The reaction is usually completed within 0.5 to 24 hours.

Examples of the compound represented by the formula (III) include hydroxylamine hydrochloride, O-methylhydroxylamine hydrochloride, O-ethylhydroxylamine hydrochloride, O-cyclopropylhydroxylamine hydrochloride, O-methylsulfonylhydroxylamine hydrochloride and so on.

2) Reaction Conditions for Hydrazonation

Examples of the reaction solvent include mixtures of a lower alcohol such as methanol, ethanol, n-butanol or isopropyl alcohol with acetic acid. Concerning the mixing ratio, it is recommended to use about 0.1 to 2.0 parts by volume of acetic acid per 10 parts by volume of the alcohol.

The reaction temperature is, for example, 0 to 150° C. and preferably recommended is 60 to 120° C. The reaction is usually completed within 0.5 to 24 hours.

Examples of the compound represented by the formula (III) include acetohydrazide, methoxycarbonyl hydrazine, methanesulfonamide, N-methylacetyl hydrazide and so on.

The liquid reaction mixture containing the compound represented by the formula (I) thus obtained also contains reagents, by-products and the like remaining therein. Thus, the compound represented by the formula (I) can be isolated through extraction and purification in accordance with publicly known methods (the same will apply the following production methods).

### Production Method 1-2

In the production method 1-2, a compound represented by the formula (I) is obtained by fusing a compound of the formula (I) wherein Z is a hydroxyl group, i.e., a compound represented by the formula (IIa) with a compound represented by the formula (IIIa).

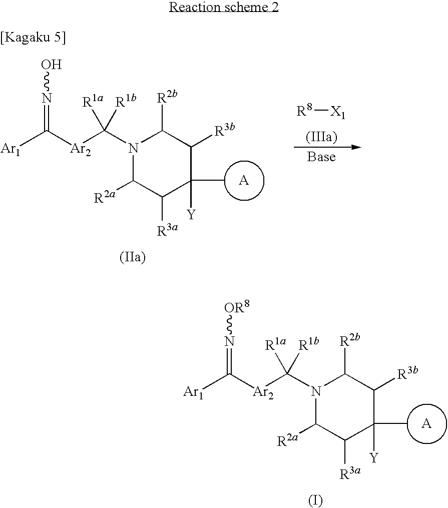

wherein X₁ represents a leaving group such as halogen, p-toluenesulfonyloxy, methanesulfonyloxy, etc.; and other symbols are each as defined above.

Namely, a compound represented by the formula (I) is obtained by condensing a compound represented by the formula (IIa) with a compound represented by the formula (IIIa) in a reaction solvent in the presence of a base.

The compound represented by the formula (IIIa) may be used in an amount of, for example, 1.0 to 2.0 mol per mol of the compound represented by the formula (IIa) and preferably recommended is 1.0 to 1.5 mol.

Examples of the reaction solvent include diethyl ether, tetrahydrofuran (hereinafter referred to as “THF”), 1,4-dioxane (hereinafter referred to as “dioxane”), dimethylformamide (hereinafter referred to as “DMF”), dimethyl sulfoxide (hereinafter referred to as “DMSO”) and so on.

Examples of the base include inorganic bases such as sodium carbonate, sodium hydrogencarbonate, potassium carbonate, potassium hydrogencarbonate, lithium carbonate and so on.

The reaction temperature is, for example, 0 to 100° C. and preferably recommended is 0 to 65° C. The reaction is usually completed within 0.5 to 24 hours.

Examples of the compound represented by the formula (IIIa) include:

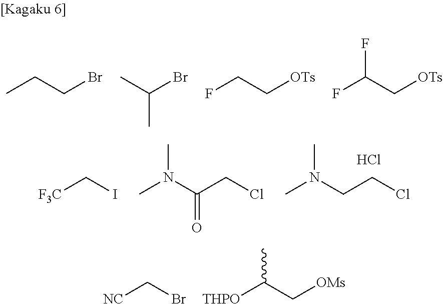

wherein Ts represents p-toluenesulfonyl; and Ms represents methanesulfonyl; and so on.

### Production Method 1-3

The production method 1-3 is a method of producing a compound represented by the formula (I) by using a compound represented by the formula (IVb) as the starting material.

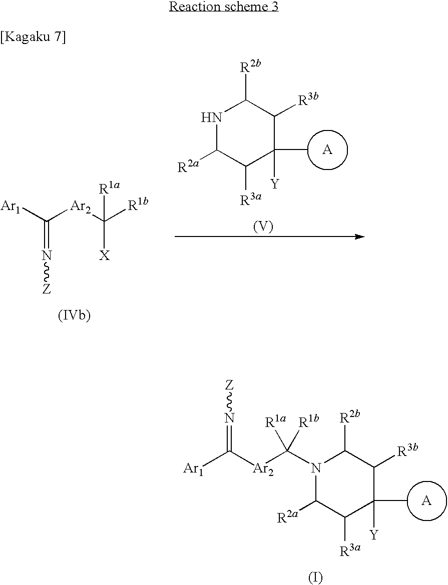

wherein X represents a leaving group such as a halogen atom, a benzenesulfonyloxy group, a p-toluenesulfonyloxy group or a methanesulfonyloxy group; and other symbols are each as defined above.

A compound represented by the formula (I) is obtained by reacting a compound represented by the formula (IVb) with a compound represented by the formula (V) in a reaction solvent preferably in the presence of a base.

The compound represented by the formula (V) may be used in an amount of, for example, 1.0 to 1.5 mol per mol of the compound represented by the formula (IVb) and preferably recommended is 1.0 to 1.3 mol.

Examples of the base include inorganic bases such as sodium carbonate, sodium hydrogencarbonate, potassium carbonate, potassium hydrogencarbonate, lithium carbonate and so on; and organic amines such as trimethylamine, triethylamine, diisopropylethylamine, pyridine and so on. The base may be used in an amount of, for example, 1.0 to 5.0 mol per mol of the compound represented by the formula (IVb) and preferably recommended is 1.1 to 1.5 mol.

Examples of the reaction solvent include halogenated hydrocarbons such as methylene chloride, chloroform, carbon tetrachloride and so on; ethers such as diethyl ether, THF, dioxane and so on; DMF, DMSO and so on.

The reaction temperature is, for example, 0 to 100° C. and preferably recommended is 10 to 40° C. The reaction is usually completed within 1 to 24 hours.

The compound represented by the formula (V) can be prepared by the methods described in WO 2004/069798 and WO 2004/064762. It is also possible to prepare the compound by the methods as will be described in Examples hereinafter.

### Production Method 2-1

The production method 2-1 is a method of producing a compound represented by the formula (II) or the formula (IIa).

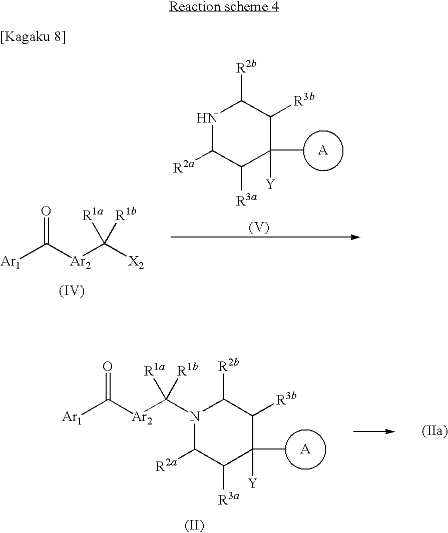

wherein X₂ has the same meaning as X₁; and other symbols are each as defined above.

A compound represented by the formula (II) is obtained by fusing a compound represented by the formula (IV) with a compound represented by the formula (V) in a reaction solvent.

The compound represented by the formula (V) may be used in an amount of, for example, 1.0 to 2.0 mol per mol of the compound represented by the formula (IV) and preferably recommended is 1.0 to 1.5 mol.

Examples of the reaction solvent include halogenated hydrocarbons such as chloroform, methylene chloride, carbon tetrachloride and so on; ethers such as THF, diethyl ether, dioxane and so on; DMF, DMSO and so on.

It is preferable to conduct the reaction in the presence of a base. Examples of the base include inorganic bases such as sodium carbonate, sodium hydrogencarbonate, potassium carbonate, potassium hydrogencarbonate, lithium carbonate and so on; and organic amines such as trimethylamine, triethylamine, diisopropylethylamine, pyridine and so on.

In the case of using a base, the amount of the base may be, for example, 1.0 to 5.0 mol per mol of the compound represented by the formula (V) and preferably recommended is 1.1 to 2.0 mol.

The reaction temperature is, for example, 0 to 100° C. and preferably recommended is 10 to 30° C. The reaction is usually completed within 0.5 to 24 hours.

The compound represented by the formula (IIa) can be prepared by using a compound represented by the formula (II) as the starting material and reacting it with hydroxylamine in accordance with the production method 1-1.

### Production Method 3-1

The production method 3-1 is a method of producing a compound represented by the formula (IVb).

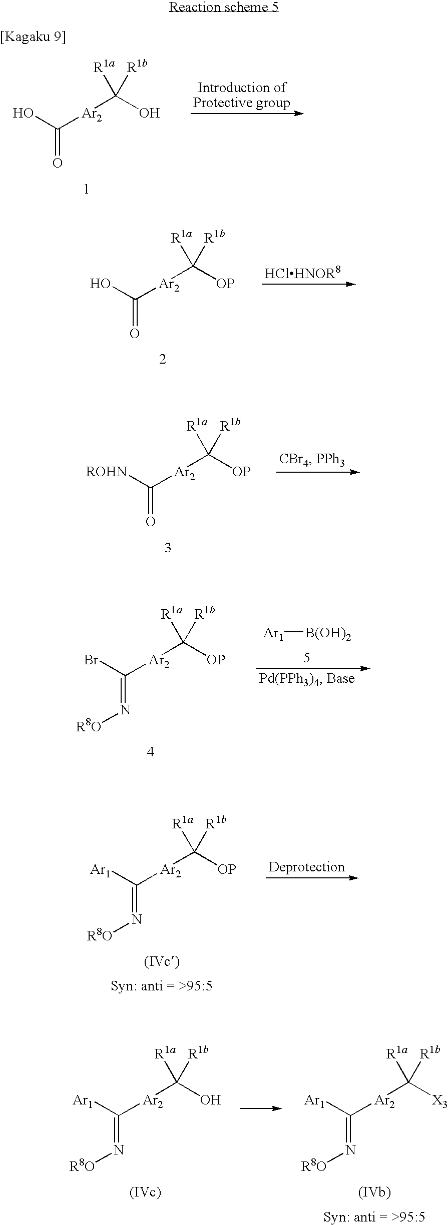

wherein P represents a hydroxyl-protective group; X₃ has the same meaning as X₁; and other symbols are each as defined above.

The hydroxyl group in a compound 1 is protected by a publicly known method to give a compound 2. Examples of the protective group include an acetyl group, a t-butyldimethylsilyl group and so on.

Subsequently, the compound 2 is condensed with an O-alkylhydroxylamine (for example, methylhydroxylamine hydrochloride, ethylhydroxylamine hydrochloride, 2-fluoroethylhydroxylamine hydrochloride, 2-trimethylsilylethylhydroxylamine hydrochloride or the like) at room temperature to give a compound 3.

Subsequently, the compound 3 is reacted under reflux in a solvent such as acetonitrile in the presence of tetrabromomethane and triphenylphosphine to give a compound 4.

Tetrabromomethane may be used in an amount of, for example, 1.5 to 3.0 mol per mol of the compound 3 and preferably recommended is 1.5 mol. On the other hand, triphenylphosphine may be used in an amount of, for example, 1.5 to 2.0 mol per mol of the compound 3 and preferably recommended is 2.0 mol.

The compound 4 thus obtained is reacted with a compound 5 in a solvent such as toluene or the like in the presence of tetrakis(triphenylphosphine)palladium and a base to give a compound represented by the formula (IVc′). Although the compound represented by the formula (IVc′) can be obtained while sustaining the stereochemistry of the compound 4, the (E) and (Z) styles vary depending on the types of Ar₁ and Ar₂. For the sake of convenience, an isomer having Ar₁ and an oxime substituent in the same side concerning the double bond is referred herein to as a syn isomer while one having in the opposite side is referred to as an anti isomer.

Examples of the base include sodium carbonate, potassium carbonate, sodium hydrogencarbonate, sodium hydroxide, potassium hydroxide and so on.

The compound 5 may be used in an amount of, for example, 1.5 to 2.0 mol per mol of the compound 4 and preferably recommended is 2.0 mol.

Tetrakis(triphenylphosphine)palladium may be used in an amount of, for example, 0.05 to 0.10 mol per mol of the compound 4 and preferably recommended is 0.05 mol. On the other hand, the base may be used in an amount of, for example, 1.5 to 2.0 mol per mol of the compound 4 and preferably recommended is 2.0 mol.

The reaction temperature is, for example, 45 to 100° C. and preferably recommended is 80° C. The reaction is usually completed within 8 to 24 hours.

Subsequently, the hydroxyl-protective group P of the compound represented by the formula (IVc′) is removed by a publicly known method to give a compound represented by the formula (IVc). Next, a leaving group (for example, a methanesulfonyloxy group, a p-toluenesulfonyloxy group or the like) is introduced into the hydroxyl group of the compound represented by the formula (IVc) to give a compound represented by the formula (IVb).

The introduction/removal of the protective group may be carried out by referring to Protective Groups in Organic Synthesis as will be described hereinafter.

Examples of the compound 1 include:

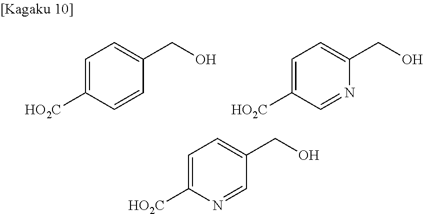

and so on, while examples of the compound 5 include:

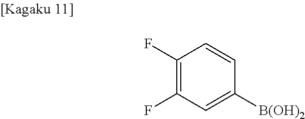

and so on.

### Production Method 3-2

The production method 3-2 is a method of producing a compound represented by the formula (IV) or the formula (IVb′).

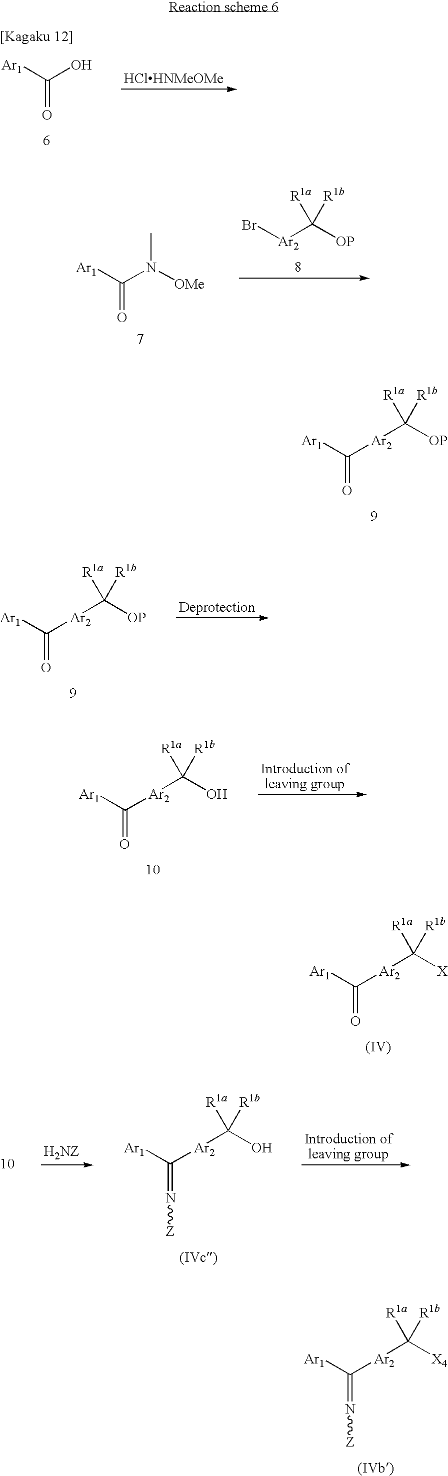

wherein X₄ has the same meaning as X₁; and other symbols are each as defined above.

Namely, a compound 6 is condensed with N-methoxy-N-methylamine hydrochloride at room temperature to give a compound 7. Subsequently, the compound 7 is reacted with a compound 8 in the presence of a base such as n-butyllithium, isopropylmagnesium chloride or the like at −78 to 0° C. to give a compound 9. The protective group of the compound 9 is removed by using tetrabutylammonium fluoride or an acid (for example, p-toluenesulfonic acid) to give a compound 10 and then the hydroxyl group of the compound 10 is converted into a leaving group (for example, mesyl chloride/triethylamine) to give a compound represented by the formula (IV). The compound 10 is reacted in accordance with the production method 1-1 to give a compound represented by the formula (IVc″) and then the hydroxyl group is converted into a leaving group (for example, mesyl chloride/triethylamine). Thus, a compound represented by the formula (IVb′) can be obtained.

Examples of the compound 6 include:

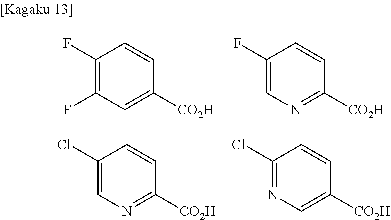

and so on. Examples of the compound 8 include:

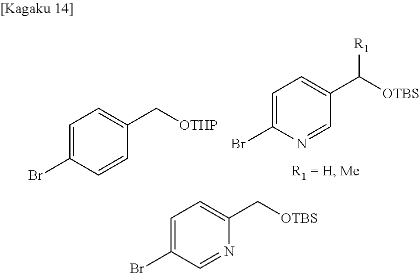

wherein HP represents tetrahydropyranyl; and TBS represents t-butylmethylsilyl; and so on.

### Production Method 3-3

The production method 3-3 is another method of producing a compound represented by the formula (IV).

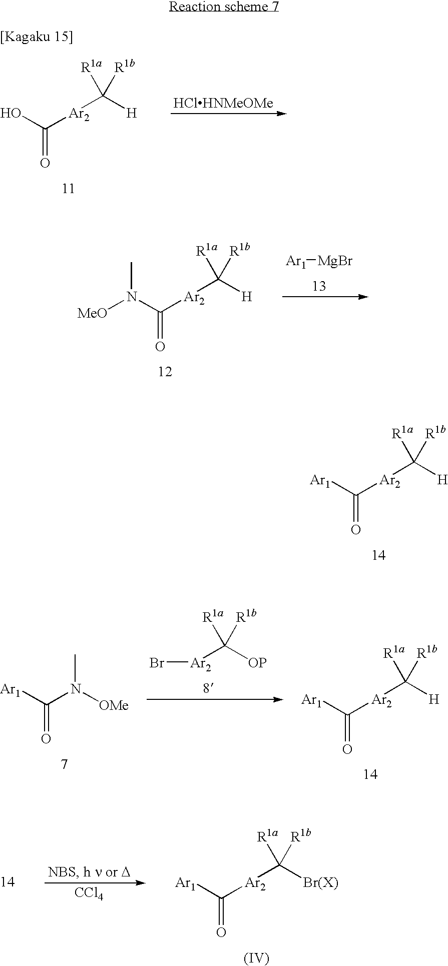

wherein each symbol is as defined above.

A compound 11 is fused with N-methoxy-N-methylamine hydrochloride at a room temperature to give a compound 12. Then, the compound 12 is reacted with a Grignard reagent 13 at 0 to 25° C. to give a compound 14.

In reacting the compound 12 with compound 13, the compound 13 is used in an amount of, for example, 1.0 to 1.5 mol per mol of the compound 12.

The reaction is conducted in an organic solvent such as THF, diethyl ether or the like. The reaction temperature is, for example, −78 to 0° C. and the reaction is usually completed within 2 to 12 hours.

On the other hand, a compound 14 can be obtained by fusing the compound 7 with the compound 8′.

That is, the compound 14 is obtained by reacting the compound 7 with the compound 8′ in a solvent such as THF, diethyl ether or the like in the presence of a base such as n-butyllithium, isopropylmagnesium chloride or the like at −78 to 0° C.

Subsequently, the compound 14 thus obtained is reacted with N-bromosuccinimide (NBS) in carbon tetrachloride under photo irradiation to give a compound represented by the formula (IV).

The reaction temperature is, for example, 60 to 80° C. and the reaction is usually completed within 4 to 24 hours.

Examples of the compound 11 include:

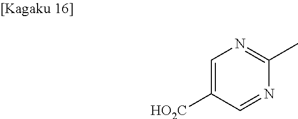

and so on, while examples of the compound 8′ include:

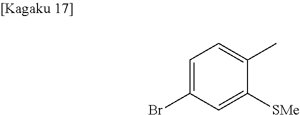

and so on.

Examples of the compound 13 include phenylmagnesium bromide, 4-fluorophenylmagnesium bromide, 3,4-difluorophenylmagnesium bromide and so on.

### Production Method 4

The production method 4 is another method of producing a compound represented by the formula (I) via a compound represented by the formula (IVa).

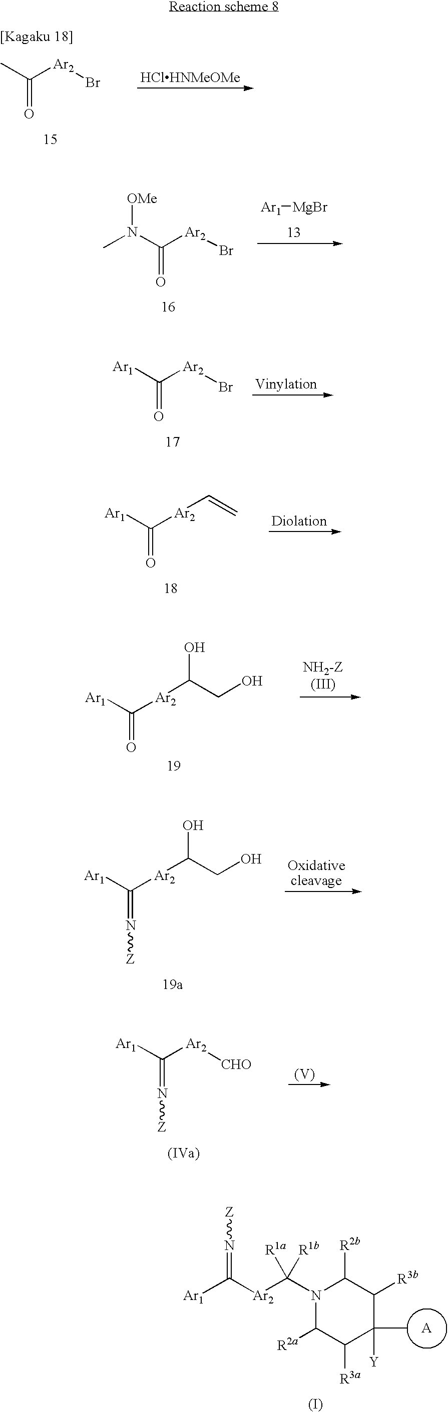

wherein each symbol is as defined above.

A compound 15 is fused with N-methoxy-N-methylamine hydrochloride at a room temperature to give a compound 16. Then, the compound 16 is reacted with a Grignard reagent 13 to give a compound 17. Subsequently, the compound 17 and potassium vinyltrifluoroborate are vinylated by conducting a cross-coupling reaction using PdCl₂dppf ([1,1′-bis(diphenylphosphino)ferrocene]palladium (II) dichloride) to give a compound 18.

In the vinylation as described above, potassium vinyltrifluoroborate is used in an amount of, for example, 1.0 to 2.0 mol per mol of the compound 17, while Pdcl₂dppf is used in an amount of, for example, 0.01 to 0.10 mol per mol of the compound 17.

The vinylation is usually conducted in an organic solvent such as n-butyl alcohol, isopropyl alcohol or the like. The reaction temperature is, for example, 80 to 120° C. and the reaction is usually completed within 1 to 4 hours.

Subsequently, the compound 18 is diolated with the use of osmium tetraoxide and N-methylmorpholine-N-oxide to give a compound 19.

Osmium tetraoxide is used in an amount of, for example, 0.01 to 0.05 mol per mol of the compound 18, while N-methylmorpholine-N-oxide is used in an amount of, for example, 1.0 to 1.5 mol per mol of the compound 18.

The diolation is conducted usually in a solvent mixture such as acetone-water, acetonitrile-water or the like. The reaction temperature is, for example, 20 to 30° C. and the reaction is usually completed within 1 to 24 hours.

Then, the compound 19 is reacted in accordance with the production method 1-1 to give a compound 19a. Subsequently, the diol moiety of the compound 19a is subjected to oxidative cleavage by a publicly known method with the use of sodium periodate. Thus, a compound represented by the formula (IVa) is obtained.

Subsequently, the compound represented by the formula (IVa) is reacted with a compound represented by the formula (V) in an organic solvent in the presence of a reducing agent.

In this reaction, the compound represented by the formula (IVa) and the compound represented by the formula (V) are usually employed at an equimolar ratio or either is employed in small molar excess.

Examples of the reducing agent include sodium cyanoborohydride, sodium triacetoxyborohydride, zinc biscyanoborohydride, nickel biscyanoborohydride and so on.

The reducing agent is used in an amount of, for example, 1.0 mol to molar excess per mol of the compound represented by the formula (IVa) and preferably recommended is 1.0 to 5.0 mol.

Examples of the reaction solvent include alcohols such as methanol, ethanol, propanol and so on; ethers such as diethyl ether, THF, dioxane and so on; halogenated hydrocarbons such as methylene chloride, chloroform, dichloroethane and so on; acetonitrile, etc.: and mixtures of the same.

The reaction temperature is, for example, −20 to 100° C. and preferably recommended is 0° C. to room temperature. The reaction is usually completed within 5 minutes to 24 hours, preferably within 1 to 6 hours.

As the compound 15, use can be made of a commercially available reagent. Alternatively, it can be prepared in accordance with the methods described in Examples.

### Production Method 5

The production method 5 is a method of producing a compound represented by the formula (V).

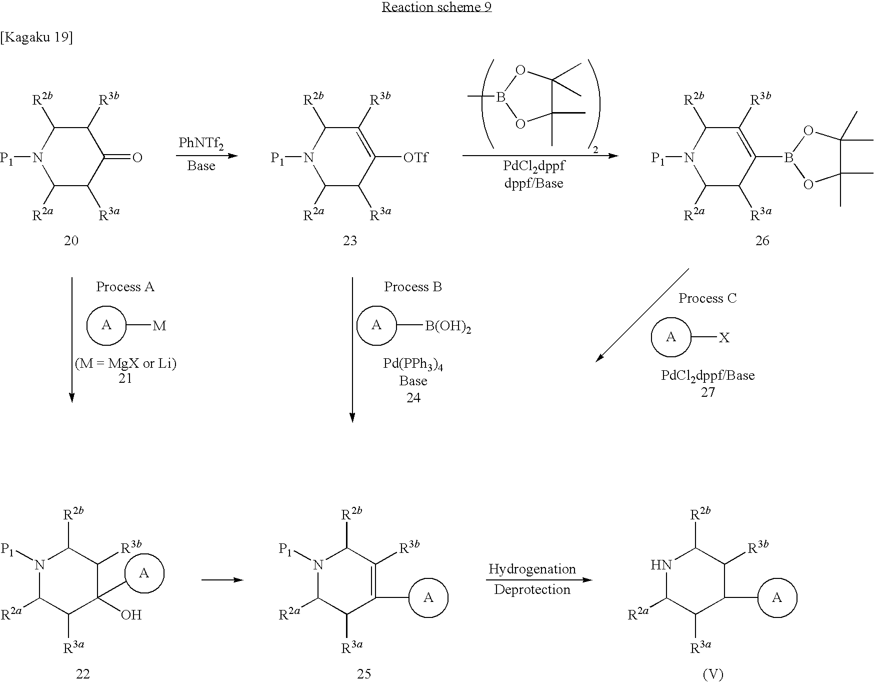

wherein P¹ represents an amino-protective group; and other symbols are each as defined above.

Process A

A compound 20 is reacted in an organic solvent with a compound 21 to give a compound 22. The compound 21 is used in an amount of, for example, 1.0 to 2.0 mol per mol of the compound 20 and preferably recommended is 1.0 to 1.2 mol.

Examples of the organic solvent include ethers such as THF, diethyl ether, dioxane and so on.

The reaction temperature is, for example, −78 to 20° C. and preferably recommended is −78° C. The reaction is usually completed within 1 to 12 hours.

Subsequently, the compound 22 is reacted with methanesulfonyl chloride in an organic solvent in the presence of dimethylaminopyridine to give a compound 25.

Subsequently, the compound 25 is hydrated in accordance with a publicly known method and, if necessary, deprotected by a method as will be described hereinafter. Thus, a compound represented by the formula (V) is obtained.

Examples of the compound 20 include:

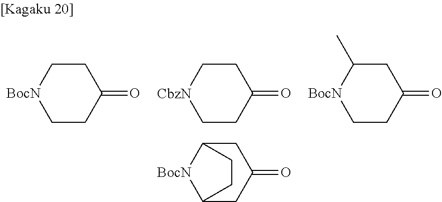

wherein Boc represents t-butyloxycarbonyl; and Cbz represents benzyloxycarbonyl; and so on, while examples of the compound 21 include:

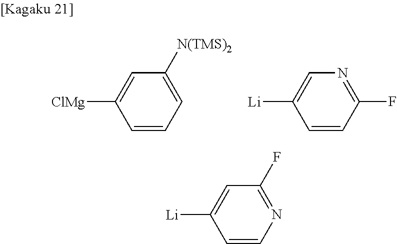

wherein TMS represents trimethylsilyl; and so on.

Process B

The compound 20 is reacted with N,N-phenylbis(trifluoromethanesulfonimide) (hereinafter sometimes abbreviated as “PhNTf₂”) in an organic solvent in the presence of a base to give a compound 23.

N,N-phenylbis(trifluoromethanesulfonimide) is used in an amount of, for example, 1.0 to 2.0 mol per mol of the compound 20 and preferably recommended is 1.0 mol.

Examples of the base include lithium diisopropylamide, lithium bishexamethyl disilazide and so on. The base is used in an amount of, for example, 1.0 to 2.0 mol per mol of the compound 20 and preferably recommended is 1.2 mol.

Examples of the organic solvent include ethers such as THF, diethyl ether, dioxane and so on.

The reaction temperature is, for example, −78 to 20° C. and preferably recommended is −78 to 0° C. The reaction is usually completed within 1 to 4 hours.

Subsequently, the compound 23 is fused with a compound 24 together with tetrakis(triphenylphosphine)palladium in an organic solvent in the presence of a base to give a compound 25.

The compound 24 is used in an amount of, for example, 1.0 to 2.0 mol per mol of the compound 23 and preferably recommended is 1.1 mol.

Tetrakis(triphenylphosphine)palladium is used in an amount of, for example, 0.05 to 0.20 mol per mol of the compound 23 and preferably recommended is 0.05 mol.

Examples of the base include inorganic bases such as sodium carbonate, sodium hydrogencarbonate, potassium carbonate, potassium hydrogencarbonate, lithium carbonate and so on. The base is used in an amount of, for example, 1.0 to 2.0 mol per mol of the compound 23 and preferably recommended is 1.2 mol.

Examples of the organic solvent include ethers such as dimethoxyethane, THF, diethyl ether, dioxane and so on; DMF, DMSO, etc.

The reaction temperature is, for example, 50 to 120° C. and preferably recommended is 80° C. The reaction is usually completed within 12 to 24 hours.

The compound 25 thus obtained can be converted into a compound represented by the formula (V) in accordance with the method of process A.

Examples of the compound 24 include:

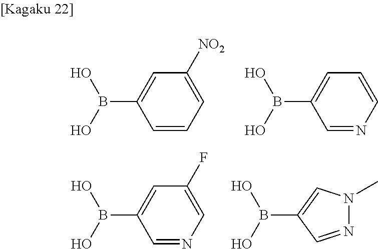

and so on.

Process C

The compound 23 is reacted with bis(pinacolate)diboron in an organic solvent in the presence of a base, PdCl₂dppf and dppf (1,1′-bis(diphenylphosphino)ferrocene) to give a compound 26.

The reaction as described above can be carried out by the method described in Tetrahedron Letters, Vol. 41, 3705 (2000). Alternatively, the reaction can be conducted by the methods as will be described in Examples hereinafter.

Subsequently, the compound 26 is reacted with a compound 27 in an organic solvent in the presence of PdCl₂dppf to give a compound 25.

The compound 26 is used in an amount of, for example, 1.0 to 2.0 mol per mol of the compound 27 and preferably recommended is 1.1 mol.

PdCl₂dppf is used in an amount of, for example, 0.05 to 0.20 mol per mol of the compound 27 and preferably recommended is 0.05 mol.

Examples of the organic solvent include DMF, DMSO and so on.

The reaction temperature is, for example, 50 to 120° C. and preferably recommended is 80° C. The reaction is usually completed within 12 to 24 hours.

Examples of the compound 27 include:

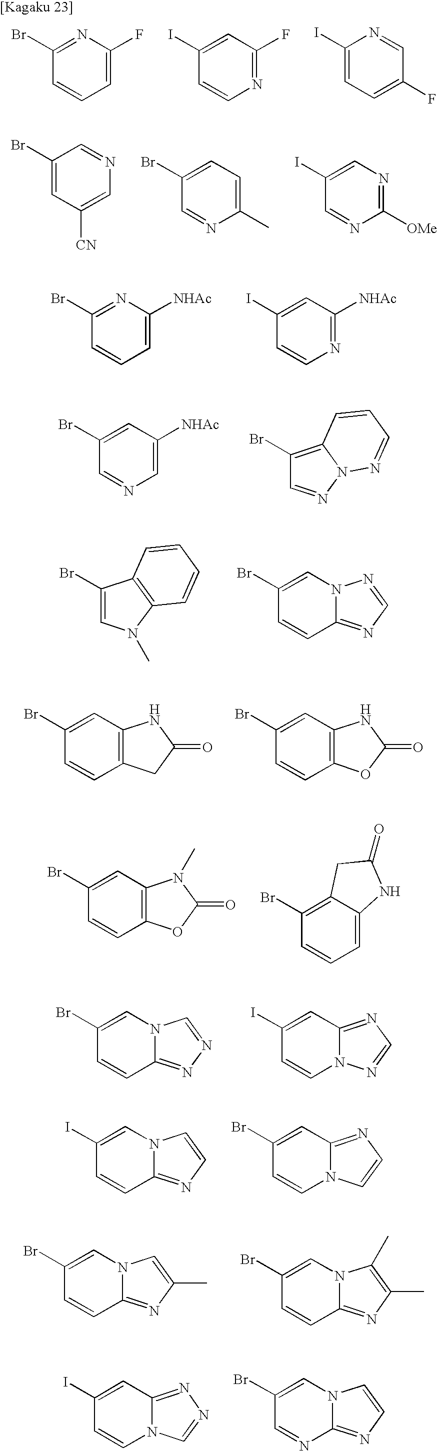

and so on.

The compound 25 thus obtained can be converted into a compound represented by the formula (V) by performing reaction in accordance with the method of process A.

When a reactant material in the above-described production method contains an amino group, an imino group, a hydroxyl group, a carboxyl group, an oxo group, a carbonyl group or the like not participating in the reaction, such amino group, imino group, hydroxyl group, carboxyl group, oxo group or carbonyl group may be protected with an appropriate amino-protective group, a hydroxyl-protective group, a carboxyl-protective group, an oxo-protective group or a carbonyl-protective group. Then, the reactions in the production method as described above can be conducted and, after the completion of the reactions, the protective group can be removed.

Means for introducing and removing protective groups differ depending on kind of the protective groups and stability of target compounds. For example, the introduction or removal may be conducted in accordance with the methods described in literature [cf. Protective Groups in Organic Synthesis, T. W. Greene, John Wiley & Sons Co., (1981)] or those analogous thereto, by, e.g., solvolysis using an acid or a base, i.e., a method using, for example, from 0.01 mole to a large molar excess of an acid, preferably trifluoroacetic acid, formic acid, hydrochloric acid or the like; or from equimolar to a large molar excess of a base, preferably potassium hydroxide, calcium hydroxide or the like, that acts on the object compound; chemical reduction using a hydrogenated metal complex; catalytic reduction using a palladium-on-carbon catalyst or a Raney nickel catalyst, and so on.

The amino- or imino-protective group is not particularly restricted so long as it has the aimed function. Examples thereof include aralkyl groups such as benzyl, p-methoxybenzyl, 3,4-dimethoxybenzyl, o-nitrobenzyl, p-nitrobenzyl, benzhydril, trityl and the like; C₁₋₆ alkanoyl groups such as formyl, acetyl, propionyl, butyryl, pivaloyl and the like; a benzoyl group; arylalkanoyl groups such as phenylacetyl, phenoxyacetyl and the like; C₁₋₆ alkoxycarbonyl groups such as methoxycarbonyl, ethoxycarbonyl, propyloxycarbonyl, tert-butoxycarbonyl (Boc) and the like; alkyloxycarbonyl groups such as benzyloxycarbonyl (Cbz), p-nitrobenzyloxycarbonyl, phenethyloxycarbonyl, and the like; C₁₋₆ alkylsilyl groups such as trimethylsilyl, tert-butyldimethylsilyl and the like; a tetrahydropyranyl group; a trimethylsilylethoxymethyl group; C₁₋₆alkylsulfonyl groups such as methyl sulfonyl, ethylsulfonyl groups and the like; arylsulfonyl groups such as benzenesulfonyl, toluenesulfonyl and the like. In particular, acetyl, benzoyl, tert-butoxycarbonyl, trimethylsilylethoxymethyl and methylsulfonyl groups, etc. are preferred.

The hydroxyl-protective group is not particularly restricted so long as it has the aimed function. Examples thereof include C₁₋₆ alkyl groups such as methyl, ethyl, propyl, isopropyl, tert-butyl and the like; C₁₋₆ alkylsilyl groups such as trimethylsilyl (TMS), tert-butyldimethylsilyl (TBS) and the like; C₁₋₆ alkoxymethyl groups such as methoxymethyl, 2-methoxyethoxy-methyl and the like; a tetrahydropyranyl (THP) group; a trimethylsilylethoxymethyl group; aralkyl groups such as benzyl, p-methoxybenzyl, 2,3-dimethoxybenzyl, o-nitrobenzyl, p-nitrobenzyl, trityl and the like; and acyl groups such as formyl, acetyl and the like. In particular, methyl, methoxymethyl, tetrahydropyranyl, trityl, trimethylsilylethoxymethyl, tert-butyldimethylsilyl and acetyl groups, etc. are preferred.

The carboxyl-protective group is not particularly restricted so long as it has the aimed function. Examples thereof include C₁₋₆ alkyl groups such as methyl, ethyl, propyl, isopropyl, tert-butyl and the like; C₁₋₆ haloalkyl groups such as 2,2,2-trichloroethyl and the like; C₁₋₆ alkenyl groups such as 2-propenyl; and aralkyl groups such as benzyl, p-methoxybenzyl, p-nitrobenzyl, benzhydryl, trityl and the like. In particular, methyl, ethyl, tert-butyl, 2-propenyl, benzyl, p-methoxybenzyl and benzhydryl groups, etc. are preferred.

The oxo- or carbonyl-protective group is not particularly restricted so long as it has the aimed function. Examples thereof include acetals and ketals such as ethylene ketal, dimethyl ketal, S,S′-dimethyl ketal and the like.

The compounds represented by the formula (I) thus obtained can be easily isolated and purified by commonly known separation means, for example, solvent extraction, recrystallization, column chromatography, preparative thin layer chromatography and the like.

The effect of as an MCH receptor antagonist of the compounds according to the invention can be proved by, for example, the following pharmacological test example.

### Pharmacological Test Example

### MCH Binding Inhibition Test

A cDNA sequence encoding human MCH-1R [FEBS Letters, Vol. 398, 253 (1996); Biochimica et Biophisica Acta, Vol. 1401, 216 (1998)] was cloned into a plasmid vector pEF/myc/cyto (manufactured by In Vitro Gene). The expression plasmid thus obtained was transfected into host cells CHO-K1 (American Type Culture Collection) by using lipofectAMINE PLUS reagent (manufactured by Life Technologies) to thereby give MCH-1R-expressing cells.

A membrane specimen prepared from the MCH-1R-expressing cells was incubated together with a test compound and 50 pM of [¹²⁵I]MCH (manufactured by NEN) in an assay buffer (a 50 mM Tris buffer (pH7.4) containing 10 mM of magnesium chloride, 2 mM of ethylenediaminetetracetic acid, 0.01% of bacitracin and 0.2% of bovine serum albumin) at 25° C. for 1 hour, and then filtered through a glass filter GF/C (manufactured by Whatman). After washing the glass filter with a 50 mM Tris buffer (pH7.4) containing 10 mM of magnesium chloride, 2 mM of ethylenediaminetetracetic acid and 0.04% of Tween-20, the radioactivity on the glass filter was determined. Nonspecific binding was measured in the presence of 1 μM human MCH and the 50% inhibitory concentration (IC₅₀) of the test compound to the specific binding to [¹²⁵I]MCH was determined. Table 1 indicates the results.

TABLE 1

| Example | Structure | IC₅₀ (nM) |
|---|---|---|
| 3-26 | 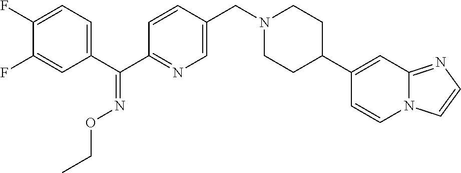 | 0.27 |
| 3-33 | 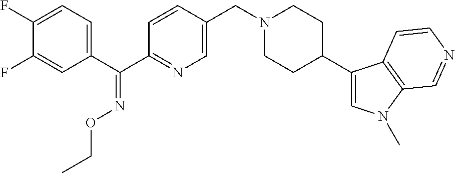 | 0.11 |
| 3-51 | 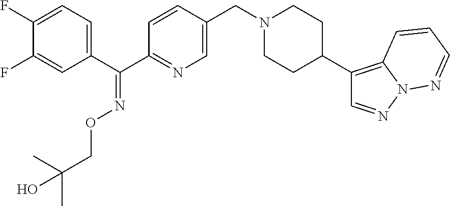 | 11.20 |
| 3-53 | 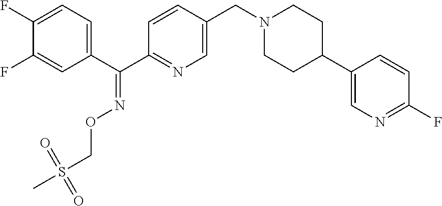 | 6.70 |
| 3-68 | 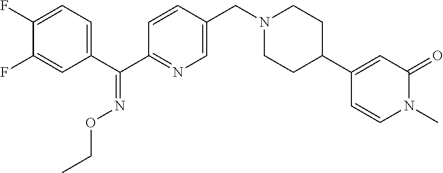 | 6.70 |
| 4-1 | 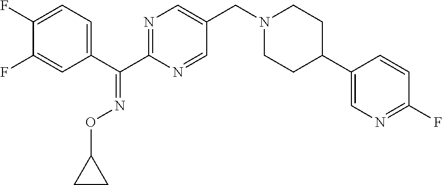 | 9.70 |

As shown above, the compounds according to the invention strongly inhibited the binding of MCH to MCH-1R, indicating an excellent effect as an MCH-1R antagonist.

Accordingly, the compounds of the invention are useful as preventives or remedies for various diseases in which MCH participates, for example, metabolic disorders such as obesity, diabetes, hormone disorder, hyperlipidemia, gout, fatty liver, and so on; circulatory diseases such as stenocardia, acute or congestive heart failure, myocardial infarction, coronary atherosclerosis, hypertension, renal diseases, electrolyte abnormality and so on; central/peripheral nervous system disorders such as bulimia, emotional disturbance, depression, anxiety, epilepsy, delirium, dementia, schizophrenia, attention-deficit hyperactivity disorder, memory impairment, sleep disorders, cognitive failure, dyskinesia, paresthesias, smell disorders, morphine tolerance, drug dependence, alcoholism and so on; reproductive disorders such as infertility, preterm labor, sexual dysfunction and so on; digestive disorders; respiratory disorders; cancer or pigmentation and, in particular, as preventives or remedies for obesity, diabetes, fatty liver, bulimia, depression and anxiety.

Medicinal Compositions Containing Compounds Represented by Formula (I)

The compounds of the invention can be administered orally or parenterally, and when formulated into preparation forms adapted for administration, these compounds are usable as medicinal compositions for preventing, treating or remedying the diseases as cited above.

In the clinical use of the compounds of the invention, pharmaceutically-acceptable additives may be added thereto to formulate various preparations in accordance with the intended administration route thereof, and then the preparations may be administered. Various additives generally used in the field of pharmaceutical compositions may be used herein, including, for example, gelatin, lactose, sucrose, titanium oxide, starch, crystalline cellulose, hydroxypropylmethylcellulose, carboxymethylcellulose, corn starch, microcrystalline wax, white petrolatum, magnesium metasilicate aluminate, anhydrous calcium phosphate, citric acid, trisodium citrate, hydroxypropylcellulose, sorbitol, sorbitan fatty acid esters, polysorbate, sucrose fatty acid esters, polyoxyethylene, hardened castor oil, polyvinylpyrrolidone, magnesium stearate, light silicic acid anhydride, talc, vegetable oils, benzyl alcohol, gum arabic, propylene glycol, polyalkylene glycols, cyclodextrin, hydroxypropylcyclodextrin and so on.

Examples of the forms of preparations comprising the compounds of the invention mixed with such additives include solid preparations such as tablets, capsules, granules, dusts and suppositories; and liquid preparations such as syrups, elixirs and injections. These preparations can be produced in any method known in the field of pharmaceutical compositions. The liquid preparations may be in such a form that is dissolved or suspended in water or in any other appropriate vehicles before using. In the case of injections, in particular, the preparation may be dissolved or suspended in a physiological saline or a glucose solution if desired, and a buffer and a preservative may be further added thereto.

Those preparations can contain the compounds of the invention in an amount of 1 to 99.9% by weight, preferably 1 to 60% by weight, based on the whole of individual pharmaceutical preparation. These preparations may also contain therapeutically active other compound(s).

In case of using the compounds of the invention as preventing or treating the above-described diseases, their dosages and administration frequency differ depending on sex, age, body weight and seriousness of symptoms of individual patients and the kind and scope of intended therapeutic effect. For oral administration, it is generally preferred to administer 0.001 to 50 mg/kg body/day of such a compound, as a single dose or several divided doses. The dosage is preferably from about 0.01 to about 25 mg/kg/day and from about 0.05 to about 10 mg/kg/day is still preferred.

The compounds of the invention can be used in combination with drugs effective for hypertension, obesity-associated hypertension, hypertension-associated diseases, cardiac hypertrophy, left ventricular hypertrophy, metabolic disorder, obesity, obesity-associated diseases and the like (hereafter referred to as “drug for combined use”). Such drugs can be administered either simultaneously, separately or successively, for preventing or treating above-described diseases. When a compound of the invention is used simultaneously with one, two or more of drugs for combined use, they may be formulated into a medical preparation suited for single administration form. However, for occasions of combination therapy, a composition containing the compound of the invention and drug(s) for combined use may be administered to a subject of medication in different packages, either simultaneously, separately or successively. Also, they may be administered at time interval(s).

The dose(s) of such drug(s) for combined use may be determinable in accordance with clinically adopted dose, which can be appropriately selected depending on the individual subject of medication, administration route, target disease, combination of drugs, and so on. The administration form of drug(s) for combined use is not particularly restricted. Namely, it is sufficient that the compound of the invention is combined with selected drug(s) for combined use before the administration.

Examples of the administration forms include: 1) administration of single preparation which is obtained by simultaneously formulating a compound of the invention together with drug(s) for combined use; 2) simultaneous administration of two kinds of preparations, which are obtained by separately formulating a compound of the invention and drug(s) for combined use, via a same administration route; 3) administration at a certain time interval, via a same administration route, of two kinds of preparations which are obtained by separately formulating a compound of the invention and drug(s) for combined use; 4) simultaneous administration of two kinds of preparations, which are obtained by separately formulating a compound of the invention and drug(s) for combined use, via different administration routes; and 5) administration of two kinds preparations, which are obtained by separately formulating the compound of the invention and drug(s) for combined use, via different administration routes at a certain time interval (e.g., administration by the order of the compound of the invention followed by the drug(s) for combined use, or vice versa). The mixing ratio of a compound of the invention to drug(s) for combined use can be appropriately selected, according to individual subject of medication, administration route, disease and so on.

Examples of the drugs for combined use which can be used in the invention include remedies for diabetes, hyperlipidemia, hypertension, obesity and the like. It is also possible to use a combination of two or more of these drugs for combined use at an adequate ratio.

Examples of the remedies for diabetes as described above include: 1) PPAR γ agonists such as glitazones [e.g., ciglitazone, darglitazone, englitazone, isaglitazone (MCC-555) and so on], pioglitazone, rosiglitazone, troglitazone, BRL49653, CLX-0921, 5-BTZD, GW-0207, LG-100641, LY-300512 and so on; 2) biganides such as metformin, buformin, phenformin and so on; 3) protein tyrosine phosphatase-1B inhibitors; 4) sulfonylureas such as acetohexamide, chloropropamide, diabinese, glibenclamide, glipizide, glyburide, glimepiride, gliclazide, glipentide, gliquidone, glisolamide, tolazamide, tolbutamide and so on; 5) meglitinides such as repaglinide, nateglinide and so on; 6) α-glucosidohydroxylase inhibitors such as acarbose, adiposine, camiglibose, emiglitate, miglitol, voglibose, pradimicin-Q, salbostatin, CKD-711, MDL-25,673, MDL-73,945, MOR14 and so on; 7) α-amylase inhibitors such as tendamistat, trestatin, A13688 and so on; 8) insulin secretion promoters such as linogliride, A-4166 and so on; 9) fatty acid oxidation repressors such as clomoxir, etomoxir and so on; 10) A2 antagonists such as midaglizole, isaglidole, deriglidole, idazoxan, earoxan, fluparoxan and so on; 11) insulin or insulin mimetics such as biota, LP-100, novarapid, insulindetemir, insulini lispro, insulin glargine, insulin zinc, Lys-Pro-insulin, GLP-1(73-7), GLP 1 amide (7-36) and so on; 12) non-thiazolidindiones such as JT-501, farglitazar and so on; and 13) PPAR α/γ dual agonists such as MK-0767, CLX-0940, GW-1536, GW-1929, GW-2433, KRP-297, L-796449, LR-90, SB219994 and so on.

Examples of the remedies for hyperlipidemia as described above include: 1) cholic acid absorbefacients such as cholesterylamine, colesevelem, colestipol, dialkylaminoalkyl derivatives of cross-dextran, Colestid™, LoCholest™, Questram™ and so on; 2) HMG-CoA reductase inhibitors such as atorvastatin, itavastatin, fluvastatin, lovastatin, pravastatin, rivastatin, rosuvastatin, simvastatin, ZD4522 and so on; 3) HMG-CoA synthesis inhibitors; 4) cholesterol absorption inhibitors such as snatol ester, β-sitosterol, sterol gluoside, ezetimibe and so on; 5) acyl coenzyme A cholesterol transacylase inhibitors such as avasimibe, eflucimibe, KY-505, SMP-709 and so on; 6) CETP inhibitors such as HT 705, torcetrapib, CP532632, BAY-63-2149, SC-591, SC-795 and so on; 7) squalene synthesis inhibitors; 8) antioxidants such as probucol; 9) PPARα agonists such as beclofibrate, benzafibrate, ciprofibrate, clofibrate, ethofibrate, fenofibrate, gemcabene, gemfibrozil, GW-7647, BM-170744, LY-518674, fabric acid derivatives [e.g., Atromid™, Lopid™, Tricor™ and so on; 10) FXR receptor antagonists such as GW-4064, SR-103912 and so on; 11) LXR receptor agonists such as GW3965, T9013137, XTCO-179628 and so on; 12) lipoprotein synthesis inhibitors such as niacin; 13) renin-angiotensin inhibitors; 14) microsomal triglyceride transport inhibitors; 15) cholic acid resorption inhibitors such as BARA 1453, SC435, PHA384640, S-435, AZD7706 and so on; 16) PPARδ agonists such as GW501516, GW590735 and so on; 17) triglyceride synthesis inhibitors; 18) MTTP inhibitors such as LAB687, CP346086 and so on; 19) low density lipoprotein receptor inducers; 20) squalene epoxidase inhibitors; 21) platelet aggregation inhibitors; and 22) 5-lipoxygenase-activating protein inhibitors such as MK-591 and so on.

Examples of the remedies for hypertension as described above include: 1) diuretic such as thiazide-type diuretic (e.g., chlorothialidon, chlorothiazide, dichlorophenamide, hydrofluorothiazide, indapamide, hydrochlorothiazide and so on), loop-type diuretic (e.g., bumetanide, ethacrynic acid, furosemide, torsemide and so on), sodium-type diuretic (e.g., amiloride, triamterene and so on); and aldosterone antagonist-type diuretic, (e.g., spironolactone, epirenone and so on); 2) β-adrenaline blockers such as acebutolol, atenolol, betaxolol, bevantolol, bisoprolol, bopindolol, carteolol, carvedilol, celiprolol, esmolol, indenolol, metaproplol, nadolol, nebivolol, penbutolol, pindolol, propanolol, sotalol, tertatolol, tilisolol, timolol and so on; 3) calcium channel blockers such as amlodipine, aranidipine, azelnidipine, barnidipine, benidipine, bepridil, cinaldipine, clevidipine, diltiazem, efonidipine, felodipine, gallopamil, isradipine, lacidipine, lemildipine, lercanidipine, nicardipine, nifedipine, nilvadipine, nimodepine, nisoldipine nitrendipine, manidipine, pranidipine, verapamil and so on; 4) angiotensin convertase inhibitors such as benazepril, captopril, cilazapril, delapril, enalapril, fosinopril, imidapril, losinopril, moexipril quinapril, quinaprilat, ramipril, perindopril, perindropril, quanipril, spirapril, tenocapril, trandolapril, zofenopril and so on; 5) neutral endopeptidase inhibitors such as omapatrilat, cadoxatril, ecadotril, fosidotril, sampatrilat, AVE7688, ER4030 and so on; 6) endothelin antagonists such as tezosentan, A308165, YM62899 and so on; 7) vasodilators such as hydrazine, clonidine, minoxidil, nicotinyl alcohol and so on; 8) angiotension II antagonists such as candesartan, eprosartan, irbesartan, losartan, pratosartan, tasosartan, telmisartan, valsartan, EXP-3137, F16828K, RNH6270 and so on; 9) α/β adrenaline blockers such as nipradilol, arotinolol, amosulalol and so on; 10) α1 blockers such as terazosin, urapidil, prazosin, bunazosin, trimazosin, doxazocin, naftopidil, indoramin, WHIP164, XEN010 and so on; 11) α2 agonists such as lofexidine, tiamenidine, moxonidine, rilmenidine, guanobenz and so on; and 12) aldosteron inhibitors; and so on.

Examples of the anti-obesity agents as described above include: 1) 5HT (serotonin) transporter inhibitors such as paroxetine, fluoxetine, fenfluramine, fluvoxamine, sertraline, imipramine and so on; 2) norepinephrine transporter inhibitors such as GW320659, desipramine, talsupram, nomifensine and so on; 3) cannabinoid 1 receptor 1 (CR-1) antagonists/inverse agonists such as rimonabant (Sanofi Synthelabo), SR-147778 (Sanofi Synthelabo), BAY-65-2520 (Bayer), SLV-319 (Sorbay) and the compounds disclosed in U.S. Pat. Nos. 5,532,237, 4,973,587, 5,013,837, 5,081,122, 5,112,820, 5,292,736, 5,624,941 and 6,028,084, WO96/33159, WO98/33765, WO98/43636, WO98/43635, WO01/09120, WO01/96330, WO98/31227, WO98/41519, WO98/37061, WO00/10967, WO00/10968, WO97/29079, WO99/02499, WO01/58869, WO02/076949, WO01/64632, WO01/64633, WO01/64634, WO03/006007, WO03/007887 and EP-658546, and so on; 4) ghrelin antagonists such as the compounds disclosed in WO01/87355, WO02/08250 and so on; 5) histamine (H3) antagonists/inverse agonists such as thioperamide, 3-(1H-imidazol-4-yl)propyl N-(pentenyl)carbonate, clobenpropit, iodophenpropit, imoproxifan, GT2395, A331440, the compounds disclosed in WO02/15905, O-[3-(1H-imidazo-4-yl)propanol carbamate, piperazin-containing H3 receptor antagonist (Lazewska, D. et al., Pharmazie, 56:927-32 (2001), benzophenone derivatives (Sasse, A. et al., Arch. Pharm. (Weinheim) 334:45-52 (2001)) substituted N-phenylcarbamates (Reidemeister, S. et al., Pharmazie, 55:83-6 (2000)), proxyphene derivatives (Sasse, A. et al., J. Med. Chem. 43:3335-43 (2000)) and so on; 6) MCH-1R antagonists such as T-226296(Takeda), SNP-7941(Synaptic) and the compounds disclosed in WO01/82925, WO01/87834, WO02/051809, WO02/06245, WO02/076929, WO02/076947, WO02/04433, WO02/51809, WO02/083134, WO02/094799, WO03/004027 and JP2001-226269A, and so on; 7) MCH-2R agonists/antagonists; 8) NPY1 antagonists such as 3-chloro-5-(1-(6-[2-(5-ethyl-4-methyl-thiazol-2-yl)-ethyl]-4-morpholinyl-4-yl-pyridin-2-ylamino)-ethyl)phenyl]carbamic acid isopropyl ester, BIBP3226, BIB03304, LY-357897, CP-671906, GI-264879, and the compounds disclosed in U.S. Pat. No. 6,001,836, WO96/14307, WO01/23387, WO99/51600, WO01/85690, WO01/85098, WO01/85173 and WO01/89528, and so on; 9) NPY5 antagonists such as 152804, GW-569180A, GW-594884A, GW-587081X, GW-548118X, FR235,208, FR226928, FR240662, FR252384, 1229U91, GI-264879A, CGP71683A, LY-377897, LY366377, PD-160170, SR-120562A, SR-120819A, JCF-104, H409/22, and the compounds disclosed in U.S. Pat. Nos. 6,140,354, 6,191,160, 6,258,837, 6,313,298, 6,337,332, 6,329,395, 340,683, 6,326,375, 6,329,395, 6,337,332 and 6,335,345, EP-01010691, EP-01044970, WO97/19682, WO97/20820, WO97/20821, WO97/20822, WO97/20823, WO98/27063, WO00/107409, WO00/185714, WO00/185730, WO00/64880, WO00/68197, WO00/69849, WO01/09120, WO01/14376, WO01/85714, WO1/85730, WO01/07409, WO01/02379, WO01/02379, WO01/23388, WO01/23389, WO01/44201, WO01/62737, WO01/62738, WO01/09120, WO02/20488, WO02/22592, WO02/48152, WO02/49648, WO02/094789 and Norman et al., J. Med. Chem. 43:42884312 (2000), and so on; 10) leptins such as human recombinant leptin (PEG-OB, Hoffman La Roche), recombinant methionyl-leptin (Amgen) and so on; 11) leptin derivatives such as the compounds disclosed in U.S. Pat. Nos. 5,552,524, 5,552,523, 5,552,522 and 5,521,283, WO96/23513, WO96/23514, WO96/23515, WO96/23516, WO96/23517, WO96/23518, WO96/23519 and WO96/23520, and so on; 12) opioid antagonists such as Nalmefene™ (Revex), 3-methoxynaltrexone, naloxone, naltrexone, the compounds disclosed in WO00/21509 and so on; 13) orexin antagonists such as SB-334867A and the compounds disclosed in WO01/96302, WO01/68609, WO02/51232, WO02/51838, WO03/023561, and so on; 14) bombesin receptor subtype 3 agonists; 15) cholecystokinin A (CCK-A) agonists such as AR-R15849, GI-181771, JMV-180, A-71378, A-71623, SR-146131, the compounds disclosed in U.S. Pat. No. 5,739,106, and so on; 16) CNTFs (ciliary neurotrophic factors) such as G1-181771 (Glaxo-SmithKline), SR146131 (Sanofi Synthelabo), butabindide, PD170,292, PD149164 (Pfizer) and so on; 17) CNTF derivatives such as axokine (Regeneron), the compounds disclosed in WO94/09134, WO98/22128 and WO99/43813, and so on; 18) growth hormone secretion receptor agonists such as NN703, hexarelin, MK-0677, SM-130686, CP-424,391, L-692,429, L-163,255, the compound disclosed in U.S. Pat. No. 6,358,951, U.S. Patent Application Nos. 2002/049196 and 2002/022637, WO01/56592 and WO02/32888, and so on; 19) serotonin receptor 2C agonists such as BVT933, DPCA37215, IK264, PNU22394, WAY161503, R-1065, YM348, the compounds disclosed in U.S. Pat. No. 3,914,250, WO02/36596, WO02/48124, WO02/10169, WO01/66548, WO02/44152, WO02/51844, WO02/40456 and WO02/40457, and so on; 20) melanocortin 3 receptor agonists; 21) melanocortin 4 receptor agonists such as CHIR86036 (Chiron), ME-10142, ME-10145 (Melacure), the compounds disclosed in WO99/64002, WO00/74679, WO01/991752, WO01/74844, WO01/70708, WO01/70337, WO01/91752, WO02/059095, WO02/059107, WO02/059108, WO02/059117, WO02/12166, WO02/11715, WO02/12178, WO02/15909, WO02/068387, WO02/068388, WO02/067869, WO03/007949 and WO03/009847, and so on; 22) monoamine resorption inhibitors such as Sibutramine™ (Meridia/Reductil) and salts thereof, the derivatives disclosed in U.S. Pat. Nos. 4,746,680, 4,806,570 and 5,436,272, US Patent Application No. 2002/0006964, WO01/27068 and WO01/62341, and so on; 23) serotonin re-uptake inhibitors such as dexfenfluramine, fluoxetine, the compounds disclosed in U.S. Pat. No. 6,365,633, WO01/27060 and WO01/162341, and so on; 24) glucagons-like peptide 1 agonists; 25) Topiramate™ (Topimax); 26) phytopharm compounds 57 (e.g., CP644,673); 27) acetyl CoA carboxylase 2 (ACC2) inhibitors; 28) β-adrenalin receptor 3 agonists such as AD9677/TAK677(Dainippon Pharmaceutical/Takeda Pharmaceutical), CL-316,243, SB418790, BRL-37344, L-796568, BMS-196085, BRL-35135A, CGPI2177A, BTA-243, W427353, Trecadrine, Zeneca D7114, SR59119A, the compounds disclosed in U.S. Pat. Nos. 5,705,515 and 5,451,677, WO01/74782 and WO02/32897, and so on; 29) diacylglycerolacyl transferase 1 inhibitors; 30) diacylglycerolacyl transferase 2 inhibitors; 31) fatty acid synthesis inhibitors such as Cerulenin, C75 and so on; 32) phosphodiesterase inhibitors such as theofylline pentoxyfylline, zaprinast, sildenafil, aminone, milrinone, cilostamide, rolipram, cilomilast and so on; 33) thyroid hormone β agonists such as KB-2611 (KaroBio BMS), the compounds disclosed in WO02/15845 and JP2000-256190A, and so on; 34) UCP (uncoupling protein)-1, 2 or 3 activators such as phytanic acid, 4-[(E)-2-(5,6,7,8-tetrahydro-5,5,8,8-tetramethyl-2-naphthalenyl)-1-propen-yl]benzoic acid (TTNPB), retinoic acid, the compounds disclosed in WO99/00123, and so on; 35) acyl estrogens such as oleoylestrone, the compounds disclosed in del Mar-Grasa, M. et al., Obesity Research, 9: 202-9 (2001) and so on; 36) glucocorticoid antagonists; 37) 11-β hydroxysteroid dehydrognase 1-type inhibitors such as BVT 3498, BVT 2733, the compounds disclosed in WO01/90091, WO 01/90090 and WO01/90092, and so on; 38) stearyl-CoA desaturase 1 inhibitors; 39) dipeptidyl peptidase IV inhibitors such as isoleucine thiazolidide, valine pyrrolidide, NVP-DPP728, AF237, P93/01, TSL225, TMC-2A/2B/2C, FE999011, P9310/K364, VIP0177, SDZ274444, the compounds disclosed in WO03/004498, WO03/004496, EP1258476, WO02/083128, WO02/062764, WO03/000250, WO03/002530, WO03/002531, WO03/002553, WO03/002593, WO03/000180 and WO03/000181, and so on; 40) lipase inhibitors such as Tetrahydro Lipstatin™ (Orlistat/Xenical), Triton WR1339, RHC80267, lipstatin, tea saponin, diethylunibelliferyl phosphate, FL-386, WAY-121898, BAY-N-3176, valilactone, esteracin, ebelactone A, ebelectone B, RHC80267, the compounds disclosed in WO01/77094, U.S. Pat. Nos. 4,598,089, 4,452,813, 5,512,565, 5,391,571, 5,602,151, 4,405,644, 4,189,438 and 4,242,453, and so on; 41) fatty acid transporter inhibitors; 42) dicarboxylate transporter inhibitors; 43) glucose transporter inhibitors; 44) phosphate transporter inhibitors; and so on.

Those combination drugs are obtained by combining a compound of the invention with one or more of the above drugs for combined use. Furthermore, the above-described combination drugs are useful for preventing, treating or remedying metabolic disorders, when combined with one or more drugs selected from the group consisting of diabetes-treating agents and hyperlipidemia-treating agents. In particular, combination containing a hypertension-treating agent together with an antiobesity agent is useful for preventing or treating metabolic disorders with synergistic effect, when a diabetes-treating agent and/or a hyperlipidemia-treating agent are added thereto.

On the other hand, the compounds according to the invention can be used combinedly with antipsychotic drugs. It has been known that antipsychotic drugs, in particular, atypical antipsychotic drugs exhibit a side effect of increasing body weight. Combined use of the compounds of the invention with the antipsychotic drugs is efficacious against this side effect. Examples of such antipsychotic drugs include olanzapine, Risperidone, quetiapine, Ziprasidone, aripiprazole, Paliperidone, Clozapine and so on. By combining the compounds of the invention with the antipsychotic drugs, metabolic parameters induced by the antipsychotic drugs (e.g., increases in blood pressure, glucose and lipids) can be improved. To the administration conditions such as dosages, subjects for medication, administration routes, administration forms and so on, the methods as described above are applicable.

### BEST MODE FOR CARRYING OUT THE INVENTION

Next, the invention will be described in greater detail with reference to the following Examples. However, it is to be understood that the invention is not restricted thereto. As a silica gel for columns, use was made of Wakogel™ C-200 (Wako Pure Chemical Industries, Ltd.). As charged silica gel columns, FLASH+™ cartridge, KP-Sil or FPNH, FLASH12+, FLASH25+S, FLASH25+M, Flash40+M, etc. (Biotage Japan) were used. In preparative thin layer chromatography, Kieselgel 60F254 (Merck) was employed. ¹HNMR spectra were measured by using JNM-AL400 (manufactured by JEOL) or MERCURY vx400 (manufactured by VARIAN). Mass spectra were measured by using Quattroll (manufactured by MICRO MASS).

### EXAMPLES

### Referential Example 1

### Synthesis of Compound 10 (Ketoalcohol 10)

### Referential Example 1-1

### Synthesis of 3,4-difluoro-N-methoxy-N-methylbenzamide

To a chlorofoim solution (115 ml) of 3,4-difluorobenzoic acid (10.0 g), N,O -dimethylhydroxylamine hydrochloride (12.3 g), 1-hydroxybenzotriazole hydrate (14.5 g), N-[3-(dimethylamino)propyl]-N′-ethylcarboximide hydrochloride (18.2 g) and triethylamine (44.5 ml) were added at 0° C. and the resultant mixture was stirred overnight at room temperature. Then water was added to the liquid reaction mixture followed by extraction with chloroform. The organic layer was washed with a saturated aqueous sodium chloride solution and dried over anhydrous sodium sulfate. The organic layer was concentrated under reduced pressure and the residue was purified by silica gel column chromatography (hexane:ethyl acetate=8:2 to 7:3). Thus, the title compound (12.8 g) was obtained as a colorless oily substance.

ESI-MS Found: m/z 202[M+H]⁺

### Referential Example 1-2

### Synthesis of 5-fluoro-N-methoxy-N-methylpyridine-2-carboxamide

The procedure of Referential Example 1-1 was followed but using 5-fluoropyridine-2-carboxylic acid (4.50 g) to give the title compound (5.88 g) as a colorless oily substance.

ESI-MS Found: m/z 185[M+H]⁺

### Referential Example 1-3

### Synthesis of 5-chloro-N-methoxy-N-methylpyridine-2-carboxamide

The procedure of Referential Example 1-1 was followed but using 5-chloropyridine-2-carboxylic acid (1.00 g) to give the title compound (1.03 g) as a colorless oily substance.

ESI-MS Found: ink 201 [M+H]⁺

### Referential Example 1-4

### Synthesis of 6-chloro-N-methyl-N-(methyloxy)-3-pyridinecarboxamide

The procedure of Referential Example 1-1 was followed but using 6-chloropyridine-3-carboxylic acid (5.00 g) to give the title compound (4.96 g) as a colorless oily substance.

ESI-MS Found: m/z 201[M+H]⁺

### Referential Example 1-5

### Synthesis of 1-(6-bromopyridin-3-yl)ethanol

To a diethyl ether solution (225 ml) of 2,5-dibromopyridine (15.0 g), 2.59 M hexane solution of (29.3 ml) of n-butyllithium was added at −78° C. and the resultant mixture was stirred for 1 hour. After adding dimethylacetamide (7.30 ml) thereto, the mixture was stirred at 0° C. for 1 hour. After adding sodium borohydride (4.79 g) and methanol (50.0 ml) at 0° C. thereto, the mixture was stirred for 1 hour. Then an aqueous acetic acid solution and a 2N aqueous sodium hydroxide solution were added to the liquid reaction mixture followed by extraction with chloroform. The organic layer was washed with water and dried over anhydrous sodium sulfate. The organic layer was concentrated under reduced pressure and the residue was purified by silica gel column chromatography (hexane:ethyl acetate=7:3 to 5:5). Thus, the title compound (7.62 g) was obtained as a brown oily substance.

ESI-MS Found: m/z 202[M+H]⁺

### Referential Example 1-6

### Synthesis of 2-bromo-5-(1-{[tert-butyl(dimethyl)silyl]oxy}ethyl)pyridine

To a DMF solution (20.0 ml) of the compound (6.00 g) obtained in Referential Example 1-5 and imidazole (4.05 g), t-butyldimethylsilyl chloride (4.93 g) was added at 0° C. and the resultant mixture was stirred overnight. After adding water, the liquid reaction mixture was extracted with diethyl ether. The organic layer was washed with water and dried over anhydrous sodium sulfate. The organic layer was concentrated under reduced pressure and the residue was purified by silica gel column chromatography (hexane:ethyl acetate-98:2 to 95:5). Thus, the title compound (7.91 g) was obtained as a colorless oily substance.

ESI-MS Found: m/z 316[M+H]⁺

### Referential Example 1-7

### Synthesis of (3,4-difluorophenyl){4-[(tetrahydro-2H-pyran-2-yloxy)methyl]phenyl}methanone

To a THF solution (50.0 ml) of 2-[(4-bromobenzyl)oxy]tetrahydro-2H-pyran (6.04 g), a 1.6 M hexane solution (19.0 ml) of n-butyllithium was added at −78° C. and the resultant mixture was stirred for 1 hour. Then, a THF solution (12.0 ml) of the amide (5.00 g) obtained in Referential Example 1-1 was added thereto at −78° C. the resultant mixture was stirred for 1 hour and then for additional 1 hour at 0° C. After adding an aqueous ammonium chloride solution, the liquid reaction mixture was extracted with ethyl acetate. The organic layer was washed with a saturated aqueous solution of sodium chloride and dried over anhydrous sodium sulfate. The organic layer was concentrated under reduced pressure and the residue was purified by silica gel column chromatography (hexane:ethyl acetate-95:5 to 8:2). Thus, the title compound (4.06 g) was obtained as a yellow oily substance.

ESI-MS Found: m/z 333[M+H]⁺

### Referential Example 1-8

### Synthesis of (3,4-difluorophenyl)[4-(hydroxymethyl)phenyl]methane

To a methanol solution (60.0 ml) of the compound (4.00 g) obtained in Referential Example 1-7, p-toluenesulfonic acid hydrate (22.9 mg) was added at room temperature and the resultant mixture was stirred for 5.5 hours. After adding an aqueous sodium hydrogencarbonate solution, the liquid reaction mixture was extracted with ethyl acetate. The organic layer was washed with a saturated aqueous solution of sodium chloride and dried over anhydrous sodium sulfate. The organic layer was concentrated under reduced pressure and the residue was purified by silica gel column chromatography (hexane:ethyl acetate-9:1 to 6:5). Thus, the title compound (2.75 g) was obtained as a white solid.

ESI-MS Found: m/z 249[M+H]⁺

### Referential Example 1-9

The following ketoalcohols 10 were obtained by using the compounds 7 and 8 (including literature-known compounds) obtained in the above Referential Example 1 and conducting reactions under the conditions as shown in Referential Examples 1-7 and 1-8 and Table 2.

TABLE 2
Synthesis of ketoalcohol 10

| 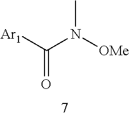 | 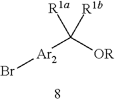 | 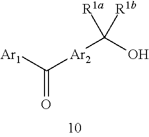 | ESI-MS |
|---|---|---|---|
| 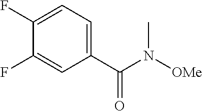 | 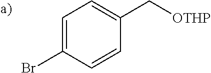 | 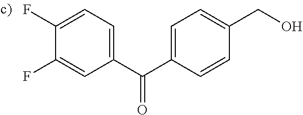 | 249 [M + H]⁺ |
| | 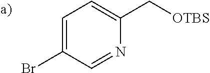 | 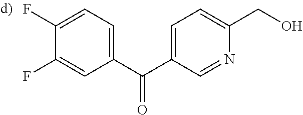 | 250 [M + H]⁺ |
| | 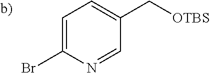 | 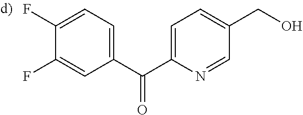 | 250 [M + H]⁺ |
| | 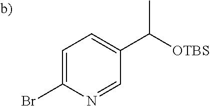 | 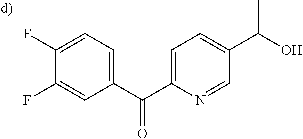 | 264 [M + H]⁺ |
| 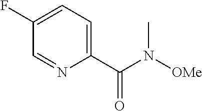 | 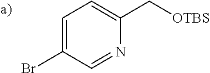 | 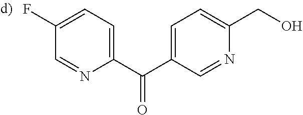 | 233 [M + H]⁺ |
| 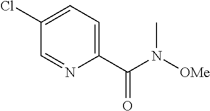 | 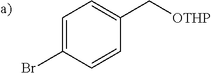 | 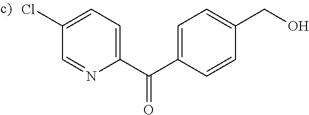 | 248 [M + H]⁺ |
| 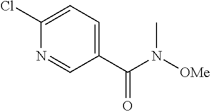 | 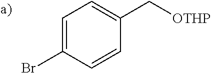 | 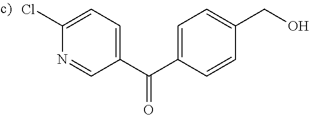 | 248 [M + H]⁺ |

conditions: a) n-BuLi, THF, −78° C., 1-2 h
b) n-BuLi, toluene-hexane, −78° C., 1 h, 0° C., 18 h
c) p-TsOH, MeOH, r.t., 1 h d) TBAF, THF, 0° C., 1-2 h

### Referential Example 2

### Synthesis of Compound Represented by Formula (IV) (Bromide (IV)

### Referential Example 2-1

### Synthesis of N-methoxy-N,5-dimethylpyrazine-2-carboxamide

The procedure of Referential Example 1-1 was followed but using 5-methylpyrazine-2-carboxylic acid (10.0 g) to give the title compound (13.2 g) as a yellow oily substance.

ESI-MS Found: m/z 182[M+H]⁺

### Referential Example 2-2

### Synthesis of (3,4-difluorophenyl)(5-methylpyrazin-2-yl)methanone

To a THF solution (50.0 ml) of the compound (3.00 g) obtained in Referential

Example 2-1, a 0.5 M THF solution (33.2 ml) of 3,4-difluorophenylmagnesium bromide was added at −78° C. and the resultant mixture was stirred for 2 hours. After heating to 0° C., an aqueous ammonium chloride solution and an aqueous sodium hydrogencarbonate solution were added thereto followed by extraction with ethyl acetate. The organic layer was washed with a saturated aqueous solution of sodium chloride and dried over anhydrous sodium sulfate. The organic layer was concentrated under reduced pressure and the residue was purified by silica gel column chromatography (hexane:ethyl acetate=8:2). Thus, the title compound (1.85 g) was obtained as a pale yellow solid.

ESI-MS Found: m/z 235[M+H]⁺

### Referential Example 2-3

### Synthesis of [5-(bromomethyl)pyrazin-2-yl](3,4-difluorophenyl)methanone

To a carbon tetrachloride solution (29.0 ml) of the compound (500 mg) obtained in Referential Example 2-2, N-bromosuccinimide (495 mg) was added at room temperature and the resultant mixture was heated and refluxed under photo irradiation for 3.5 hours. After filtrating through a Kiriyama funnel, the filtrate was concentrated under reduced pressure. Then, the residue was purified by preparative thin layer silica gel chromatography (hexane:ethyl acetate=7:3). Thus, the title compound (291 mg) was obtained as a yellow oily substance.

ESI-MS Found: m/z 313[M+H]⁺

### Referential Example 2-4

### Synthesis of N-methoxy-N,2-dimethylpyrimidine-5-carboxamide

The procedure of Referential Example 1-1 was followed but using 2-methylpyrimidine-5-carboxylic acid (3.00 g) to give the title compound (3.12 g) as a yellow oily substance.

ESI-MS Found: m/z 182[M+H]⁺

### Referential Example 2-5

### Synthesis of 4-bromo-1-methyl-2-(methylthio)benzene

To a DMF solution (15.0 ml) of 4-bromo-2-fluorotoluene (3.00 ml), sodium methylsulfide (1.84 g) was added at room temperature and the resultant mixture was stirred at 50° C. for 18 hours. After adding an aqueous sodium hydrogencarbonate solution, the liquid reaction mixture was extracted with diethyl ether. The organic layer was washed with water and a saturated aqueous solution of sodium chloride and dried over anhydrous sodium sulfate. The organic layer was concentrated under reduced pressure and the residue was purified by silica gel column chromatography (hexane:diethyl ether=200:1 to 50:1). Thus, the title compound (2.51 g) was obtained as a pale yellow oily substance.

ESI-MS Found: rah 218[M+H]⁺

### Referential Example 2-6

The following bromides (IV) were obtained by using the compounds 12 and 7 (including literature-known compounds) obtained in the above Referential Example 2 and conducting reactions under the conditions as shown in Referential Examples 1-7, 2-2 and 2-3 and Table 3.

TABLE 3
Synthesis of bromide (IV)

| 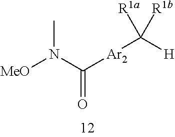 | 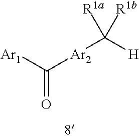 | 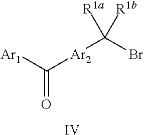 | ESI-MS |
|---|---|---|---|
| 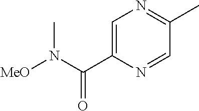 | 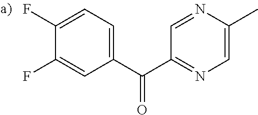 | 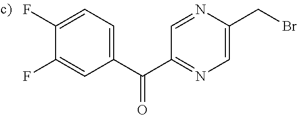 | 313 [M + H]⁺ |
| 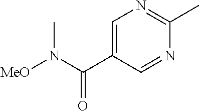 | 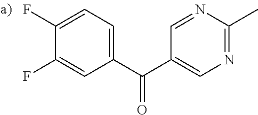 | 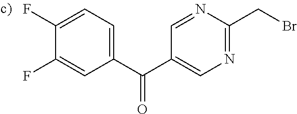 | 313 [M + H]⁺ |
| 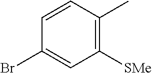 | 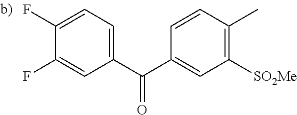 | 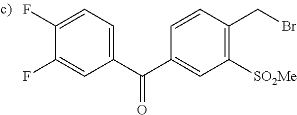 | 389 [M + H]⁺ |

conditions: a) 3,4-Difluorophenyl magnesium bromide, THF, 0° C., 1-3 h
b) (1) n-BuLi, THF, −78° C., 1 h, then 3,4-Difluoro-N-methoxy-N-methylbenzamide
(2) mCPBA, CHCl₃, 0° C., 4 h
c) NBS, h ν, CHCl₃, reflux, 2-5 h or NBS, AlBN, CHCl₃, reflux, 2-5 h

### Referential Example 3

### Synthesis of Compound Represented by Formula (V) (Amine (V))

### Referential Example 3-1

### Synthesis of tert-butyl 4-trifluoromethanesulfonyloxy-3,6-dihydro-2H-pyridin-1-yl-carboxylate

To a tetrahydrofuran solution (400 ml) of tert-butyl 4-oxy-piperidine-1-carboxylate (39.9 g), a 1.0 M tetrahydrofuran solution (220 ml) of lithium bis(trimethylsilyl)amide was added at −78° C. and the resultant mixture was stirred for 30 minutes. Next, a tetrahydrofuran solution (200 ml) of N-phenylbis(trifluoromethanesulfonimide) (71.5 g) was added thereto at −78° C. and the resultant mixture was stirred at room temperature for 3 hours. After adding water, the liquid reaction mixture was extracted with ethyl acetate. The organic layer was washed thrice with water and then once with a saturated aqueous solution of sodium chloride and dried over anhydrous sodium sulfate. The organic layer was concentrated under reduced pressure. Thus, the title compound (65.1 g) was obtained as a yellow solid.

ESI-MS Found: m/z 354[M+Na]⁺

### Referential Example 3-2

### Synthesis of benzyl 4-{[(trifluoromethyl)sulfonyl]oxy}-3,6-dihydropyridine-1(2H)carboxylate

The procedure of Referential Example 3-1 was followed but using benzyl 4-oxy-piperidine-1-carboxylate (46.7 g) to give the title compound (78.3 g) as a pale brown oily substance.

ESI-MS Found: m/z 388[M+Na]⁺

### Referential Example 3-3

### Synthesis of tert-butyl 2-methyl-4-{[(trifluoromethyl)sulfonyl]oxy}-3,6-dihydropyridine-1(2H)carboxylate

The procedure of Referential Example 3-1 was followed but using tert-butyl 2-methyl-4-oxopiperidine-1-carboxylate (1.0 g) to give the title compound (1.56 g) as a pale brown oily substance.

ESI-MS Found: m/z, 368[M+Na]⁺

### Referential Example 3-4

### Synthesis of tert-butyl 3-{[(trifluoromethyl)sulfonyl]oxy}-8-azabicyclo[3.2.1]octa-2-ene-8-carboxylate

The procedure of Referential Example 3-1 was followed but using tert-butyl 3-oxo-8-azabicyclo[3.2.1]octane-8-carboxylate (900 mg) to give the title compound (1.35 g) as a pale brown oily substance.

ESI-MS Found: m/z 358[M+H]⁺

### Referential Example 3-5

### Synthesis of tert-butyl 4-(4,4,5,5-tetramethyl-[1,3,2]dioxaborolan-2-yl)-3,6-dihydro-2H-pyridine-1-carboxylate

To a dimethyl sulfoxide solution (830 ml) of the compound (65.1 g) obtained in Referential Example 3-1, bis(pinacolate)diboron (42.1 g), potassium acetate (48.9 g), 1,1′-bis(diphenylphosphino)ferrocene (4.60 g) and [1,1′-bis(diphenylphosphino)ferrocene]palladium chloride (6.78 g) were added and the resultant mixture was stirred at 80° C. for 3 hours. After adding water, the liquid reaction mixture was extracted with ethyl acetate. The organic layer was washed with water and a saturated aqueous solution of sodium chloride and dried over anhydrous sodium sulfate. The organic layer was concentrated under reduced pressure and the residue was purified by silica gel column chromatography (hexane:ethyl acetate=90:10). The solid thus obtained was crystallized from hexane. Thus, the title compound (16.9 g) was obtained as a white solid.

ESI-MS Found: m/z 332[M+Na]⁺

### Referential Example 3-6

### Synthesis of benzyl 4-(4,4,5,5-tetramethyl-1,3,2-dioxaborolan-2-yl)-3,6-dihydropyridine-1(2H)-carboxylate

The procedure of Referential Example 3-5 was followed but using the compound (40.2 g) obtained in Referential Example 3-2 to give the title compound (24.7 g) as a white solid.

ESI-MS Found: m/z 366[M+Na]+

### Referential Example 3-7

### Synthesis of tent-butyl 2-methyl-4-(4,4,5,5-tetramethyl-1,3,2-dioxaborolan-2-yl)-3,6-dihydropyridine-1(2H)-carboxylate

The procedure of Referential Example 3-5 was followed but using the compound (1.56 g) obtained in Referential Example 3-3 to give the title compound (791 mg) as a colorless oily substance.

ESI-MS Found: m/z 346[M+Na]⁺

### Referential Example 3-8

### Synthesis of tert-butyl 3-(4,4,5,5-tetramethyl-1,3,2-dioxaborolan-2-yl)-8-azabicyclo[3.2.1]octa-2-en-8-carboxylate

The procedure of Referential Example 3-5 was followed but using the compound (1.35 g) obtained in Referential Example 3-4 to give the title compound (1.21 g) as a pale yellow solid.

ESI-MS Found: m/z 336[M+H]⁺

### Referential Example 3-9

### Synthesis of tert-butyl 6-fluoro-3′,6′-dihydro-2,4′-bipyridine-1′(2′H)-carboxylate

To an N,N-dimethylformamide solution (65 ml) of the compound (3.46 g) obtained in Referential Example 3-5, 2-bromo-6-fluoropyridine (1.82 g), potassium carbonate (4.15 g) and [1,1′-bis(diphenylphosphino)ferrocene]palladium chloride (0.73 g) were added and the resultant mixture was stirred at 80° C. for 18 hours. After adding water, the liquid reaction mixture was extracted with ethyl acetate. The organic layer was washed with a saturated aqueous solution of sodium chloride and dried over anhydrous sodium sulfate. The organic layer was concentrated under reduced pressure and the residue was purified by silica gel column chromatography (hexane:ethyl acetate-100:0 to 50:50). Thus, the title compound (0.79 g) was obtained as a green oily substance.

ESI-MS Found: m/z 301[M+Na]+

### Referential Example 3-10

### Synthesis of tert-butyl 4-(6-fluoropyridin-2-yl)piperidine-1-carboxylate

To a methanol solution (10.0 ml) of the compound (789 mg) obtained in Referential Example 3-9, 20% palladium hydroxide-carbon (400 mg) was added. After stirring the resultant mixture in a hydrogen atmosphere (1 atm) at room temperature for 5 hours, the reaction system was purged with nitrogen. The reaction mixture was filtered through celite and the filtrate was concentrated under reduced pressure. The residue was purified by silica gel column chromatography (hexane:ethyl acetate-100:0 to 50:50). Thus, the title compound (363 mg) was obtained as a colorless oily substance.

ESI-MS Found: m/z 181[M-Boc+H]⁺

### Referential Example 3-11

### Synthesis of 2-fluoro-6-piperidin-2-ylpyridine hydrochloride

To the compound (105 mg) obtained in Referential Example 3-10, a 4N hydrochloric acid-ethyl acetate solution (2.0 ml) was added and the resultant mixture was stirred at room temperature for 30 minutes. After concentrating the solvent under reduced pressure, the title compound was obtained as a while solid.

ESI-MS Found: m/z 181[M+H]+

### Referential Example 3-12

### Synthesis of 3-bromo-1-methyl-1H-pyrrolo[2,3-c]pyridine

To an N,N-dimethylformamide solution (8.8 ml) of 3-bromo-1H-pyrrolo[2,3-c]pyridine (345 mg), dimethyl sulfate (201 μl) and sodium hydride (84 mg) were added at 0° C. and the resultant mixture was stirred for 1.5 hour. After adding a saturated aqueous solution of sodium hydrogencarbonate, the liquid reaction mixture was extracted with ethyl acetate. The organic layer was washed with a saturated aqueous solution of sodium chloride and dried over anhydrous sodium sulfate. Then, the organic layer was concentrated under reduced pressure and the residue was purified by thin layer silica gel chromatography (chloroform:methanol=95:5). Thus, the title compound (73.8 mg) was obtained.

ESI-MS Found: m/z 211 [M]⁺

### Referential Example 3-13

### Synthesis of 3-bromopyrazolo[1,5-b]pyridazine

To a DMF solution (1100 ml) of the pyrazolo[1,5-b]pyridazine-3-carboxylic acid (40.0 g), sodium hydrogencarbonate (24.7 g) and N-bromosuccinimide (52.4 g) were added and the resultant mixture was stirred at room temperature for 3 hours. After adding water, the liquid reaction mixture was extracted with diethyl ether. The organic layer was washed with water and then dried over anhydrous sodium sulfate. The organic layer was concentrated under reduced pressure and the residue was purified by silica gel column chromatography (methylene chloride:hexane=5:5). Thus, the title compound (38.0 g) was obtained as a white solid.

ESI-MS Found: m/z 198[M]⁺

### Referential Example 3-14

The following amines (V) were obtained by using the compound 26 and compound 27 (including literature-known compounds) obtained in the above Referential Example 3 and conducting reactions under the conditions as shown in Referential Examples 3-9, 3-10 and 3-11 (in the case of the amine-protective group being a t-butoxycarbonyl group).

TABLE 4-1
Synthesis of amine (V)

| 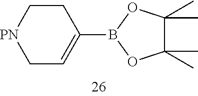 | Ar—X 27 | 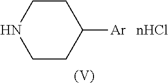 | ESI-MS |
|---|---|---|---|
| 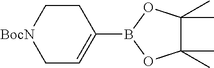 | 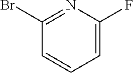 | 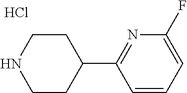 | 181 [M + H]⁺ |
| | 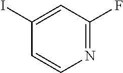 | 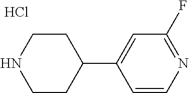 | 181 [M + H]⁺ |
| | 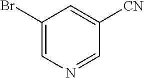 | 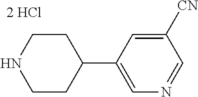 | — |
| | 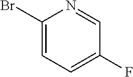 | 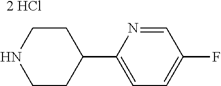 | — |
| | 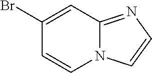 | 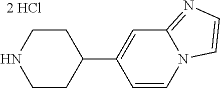 | 202 [M + H]⁺ |
| | 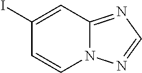 | 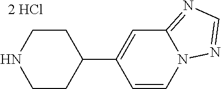 | 203 [M + H]⁺ |
| | 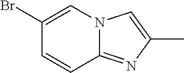 | 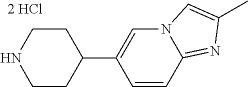 | 216 [M + H]⁺ |
| | 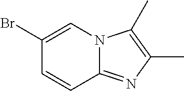 | 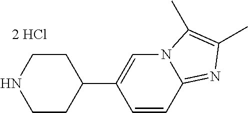 | 230 [M + H]⁺ |
| | 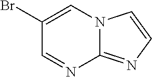 | 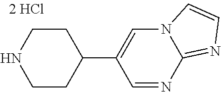 | 203 [M + H]⁺ |

TABLE 4-2
Synthesis of amine (V)

| 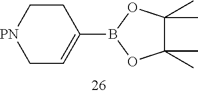 | Ar—X 27 | 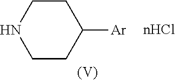 | ESI-MS |
|---|---|---|---|
| 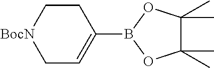 | 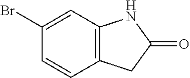 | 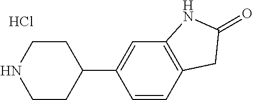 | 217 [M + H]⁺ |
| | 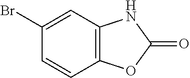 | 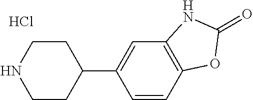 | 219 [M + H]⁺ |
| | 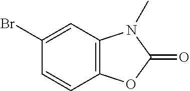 | 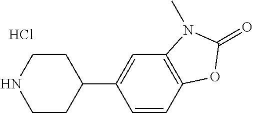 | 233 [M + H]⁺ |
| | 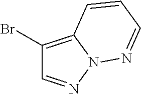 | 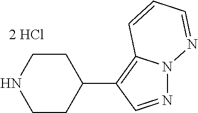 | 203 [M + H]⁺ |
| | 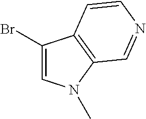 | 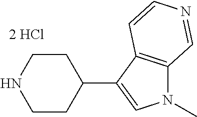 | — |
| 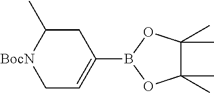 | 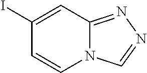 | 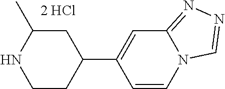 | 217 [M + H]⁺ |
| 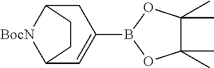 | 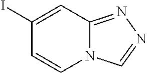 | 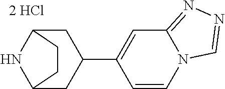 | 229 [M + H]⁺ |

TABLE 4-3
Synthesis of amine (V)

| 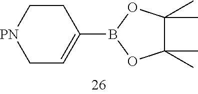 | Ar-X 27 | 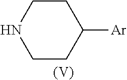 | ESI-MS |
|---|---|---|---|
| 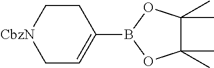 | 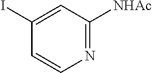 | 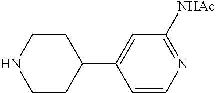 | 220 [M + H]⁺ |
| | 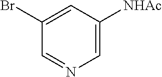 | 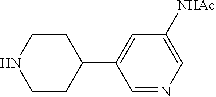 | 220 [M + H]⁺ |
| | 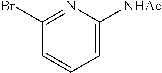 | 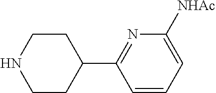 | 220 [M + H]⁺ |
| | 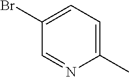 | 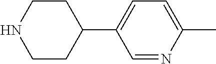 | 177 [M + H]⁺ |
| | 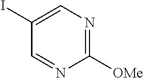 | 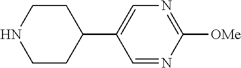 | 194 [M + H]⁺ |
| | 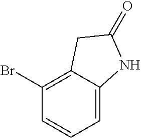 | 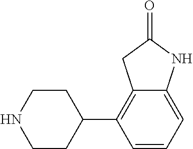 | 217 [M + H]⁺ |
| | 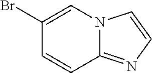 | 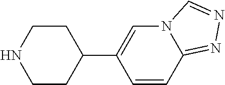 | 203 [M + H]⁺ |
| | 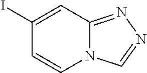 | 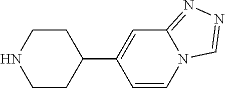 | 203 [M + H]⁺ |
| | 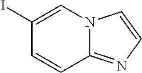 | 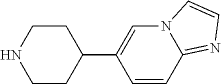 | 202 [M + H]⁺ |
| | 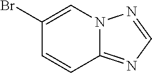 | 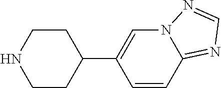 | 203 [M + H]⁺ |

### Referential Example 3-15

### Synthesis of tert-butyl 4-(2-oxo-2,3-dihydro-1H-indol-6-yl)piperidine-1-carboxylate

The procedures of Referential Examples 3-9 and 3-10 were followed but using 6-bromo-1,3-dihydro-2H-indol-2-one (1.0 g) to give the title compound (61.7 mg) as a yellow amorphous substance.

ESI-MS Found: m/z 339[M+Na]⁺

### Referential Example 3-16

### Synthesis of 1-methyl-6-piperidin-4-yl-1,3-dihydro-2H-indol-2-one hydrochloride

The procedures of Referential Examples 3-12 and 3-11 were followed but using the compound (61.7 mg) obtained in Referential Example 3-15 to give the title compound as a brown solid.

ESI-MS Found: m/z 231[M+H]⁺

### Referential Example 3-17

### Synthesis of N-methyl-N-(3-piperidin-4-ylphenyl)acetamide hydrochloride

The procedures of Referential Examples 3-12 and 3-11 were followed but using tert-butyl 4[3-(acetylamino)phenyl]piperidine-1-carboxylate (955 mg) to give the title compound (572 mg) as a white amorphous substance.

ESI-MS Found: m/z 233[M+H]⁺

### Referential Example 3-18

### Synthesis of tert-butyl 5-fluoro-3′,6′-dihydro-3,4′-bipyridine-1′(2′H)-carboxylate

To an acetonitrile solution (10 ml) of the compound (1.23 g) obtained in Referential Example 3-1, (5-fluoropyridin-3-yl)boronic acid (262 mg), tetrakistriphenylphosphine palladium (107 mg) and a 0.4 M aqueous sodium carbonate solution (10 ml) were added and the resultant mixture was stirred at 80° C. for 12 hours. After adding water, the liquid reaction mixture was extracted with ethyl acetate. The organic layer was washed with water and a saturated aqueous solution of sodium chloride and dried over anhydrous magnesium sulfate. Then, the organic layer was concentrated under reduced pressure and the residue was purified by silica gel column chromatography (hexane:ethyl acetate-100:0 to 75:25). Thus, the title compound (445 mg) was obtained as a mixture with piperidone.

ESI-MS Found: m/z 279[M+Na]⁺

### Referential Example 3-19

### Synthesis of 3-fluoro-5-piperidin-4-ylpyridine hydrochloride

The procedures of Referential Examples 3-10 and 3-11 were followed but using the compound obtained in Referential Example 3-18 to give the title compound as a pale yellow solid.

ESI-MS Found: m/z 181[M+H]⁺

### Referential Example 3-20

### Synthesis of 4-(1-methyl-1H-pyrazol-4-yl)piperidine dihydrochloride

The procedures of Referential Examples 3-18, 3-10 and 3-11 were followed but using 1-methyl-4-(4,4,5,5-tetramethyl-1,3,2-dioxabororan-2-yl)-1H-pyrazole to give the title compound as a white solid.

ESI-MS Found: m/z 166[M+H]⁺

### Referential Example 3-21

### Synthesis of tert-butyl 4-(6-fluoropyridin-3-yl)-4-hydroxypiperidine-1-carboxylate

To a diethyl ether solution (100 ml) of 5-bromo-2-fluoropyridine (5.27 g), a 1.6 M hexane solution (19.0 ml) of n-butyllithium was added at −78° C. and the resultant mixture was stirred for 10 minutes. Next, a tetrahydrofuran solution (50 ml) of t-butyl 4-oxy-piperidine-1-carboxylate (5.98 g) was added at −78° C. thereto followed by stirring for 40 minutes. After adding a saturated aqueous solution of ammonium chloride, the liquid reaction mixture was extracted with ethyl acetate. The organic layer was washed with a saturated aqueous solution of sodium chloride and dried over anhydrous sodium sulfate. Then, the organic layer was concentrated under reduced pressure and the residue was purified by silica gel column chromatography (hexane:ethyl acetate=100:0 to 0:100). Thus, the title compound (7.05 g) was obtained as a white solid.

ESI-MS Found: m/z 319[M+Na]⁺

### Referential Example 3-22

### Synthesis of tert-butyl 6-fluoro-3′,6′-dihydro-3,4′-bipyridine-1′(2′H)-carboxylate

To a chloroform solution (180 ml) of the compound (7.05 g) obtained in Referential Example 3-21, 4-dimethylaminpyridine (18.2 g) and chloromethanesulfonic acid (4.61 ml) were added at 0° C. and the resultant mixture was stirred at room temperature for 2 days. After adding an aqueous citric acid solution (32 g/250 ml), the liquid reaction mixture was extracted with ethyl acetate. The organic layer was washed successively with water, a saturated aqueous solution of sodium hydrogencarbonate and a saturated aqueous solution of sodium chloride in this order and then dried over anhydrous sodium sulfate. Then, the organic layer was concentrated under reduced pressure and the residue was purified by silica gel column chromatography (hexane:ethyl acetate=100:0 to 50:50). Thus, the title compound (6.18 g) was obtained as a yellow oily substance.

ESI-MS Found: m/z 301[M+Na]⁺

### Referential Example 3-23

### Synthesis of 2-fluoro-5-piperidin-4-ylpyridine hydrochloride

The procedures of Referential Examples 3-10 and 3-11 were followed but using the compound obtained in Referential Example 3-22 to give the title compound as a white solid.

ESI-MS Found: m/z 181[M+H]⁺

### Referential Example 3-24

### Synthesis of 4-(6-fluoropyridin-3-yl)piperidin-4-ol hydrochloride

The procedure of Referential Example 3-11 was followed but using the compound obtained in Referential Example 3-21 to give the title compound as a white solid.

ESI-MS Found: m/z 197[M+H]⁺

### Referential Example 3-25

### Synthesis of benzyl 4-(3-aminophenyl)-4-hydroxypiperidine-1-carboxylate

To a tetrahydrofuran solution (20 ml) of benzyl 4-oxy-piperidine-1-carboxylate (1.17 g), a 1 M tetrahydrofuran solution (19.0 ml) of {3-[bis(trimethylsilyl)amino]phenyl}magnesium chloride was added at room temperature and the resultant mixture was stirred for 1.5 hour. After adding a 2 N aqueous hydrochloric acid solution, a 15% aqueous sodium hydroxide solution was added to the liquid reaction mixture followed by extraction with ethyl acetate. The organic layer was washed with a saturated aqueous solution of sodium chloride and dried over anhydrous sodium sulfate. Then, the organic layer was concentrated under reduced pressure and the residue was purified by silica gel column chromatography (hexane:ethyl acetate=100:0 to 0:100). Thus, the title compound (0.82 g) was obtained as an orange amorphous substance.

ESI-MS Found: m/z 309[M−H₂O+H]⁺

### Referential Example 3-26

### Synthesis of benzyl 4-[3-(acetylamino)phenyl]-4-hydroxypiperidine-1-carboxylate

To an N,N-dimethylformamide solution (10 ml) of the compound (821 mg) obtained in Referential Example 3-25, acetic acid (159 μl), 2-(1H-7-azabenzotriazol-1-yl)-1,1,3,3-tetramethyluronium hexafluorophosphate (1.05 g) and diisopropylethylamine (878 μl) were added at 0° C. and the resultant mixture was stirred at room temperature for 17 hours. After adding water, the liquid reaction mixture was extracted with ethyl acetate. The organic layer was washed with a saturated aqueous solution of sodium chloride and dried over anhydrous sodium sulfate. Then, the organic layer was concentrated under reduced pressure and the residue was purified by silica gel column chromatography (chloroform:methanol=100:0 to 90:10). Thus, the title compound (1.07 g) was obtained as an orange oily substance.

ESI-MS Found: m/z 369[M+H]⁺

### Referential Example 3-27

### Synthesis of N-[3-(4-hydroxypiperidin-4-yl)phenyl]acetamide

The procedure of Referential Example 3-10 was followed but using the compound (1.07 g) obtained in Referential Example 3-26 to give the title compound (657 mg) as a white amorphous substance.

ESI-MS Found: m/z 235[M+H]⁺

### Referential Example 3-28

### Synthesis of tert-butyl 4-(2-fluoropyridin-4-yl)piperidine-1-carboxylate

The procedures of Referential. Examples 3-21, 3-22 and 3-10 were followed but using 2-fluoro-4-iodopyridine (44.6 g) to give the title compound (15.2 g) as a pale yellow oily substance.

ESI-MS Found: m/z 281[M+H]⁺

### Referential Example 3-29

### Synthesis of tert-butyl 4-(2-oxo-1,2-dihydropyridin-4-yl)piperidine-1-carboxylate

To the compound (12.0 g) obtained in Referential Example 3-28, a 3N aqueous hydrochloric acid solution (143 ml) was added and the resultant mixture was stirred at 100° C. for 7 hours. Then, the liquid reaction mixture was cooled to 0° C. After adding sodium hydroxide (19.8 g), 1,4-dioxane (143 ml) and di-tert-butyl Bicarbonate (10.3 g), the resultant mixture was stirred at room temperature for 15 hours. Then, 1,4-dioxane was distilled off under reduced pressure and the white solid thus formed was collected by filtration. The obtained solid was washed with water. Thus, the title compound (12.9 g) was obtained as a white solid.

ESI-MS Found: m/z 301 [M+Na]⁺

### Referential Example 3-30

### Synthesis of 4-piperidin-4-ylpyridin-2(1H)-one hydrochloride

To the compound obtained in Referential Example 3-28, a 3N aqueous hydrochloric acid solution was added and the resultant mixture was stirred at 100° C. for 7 hours. Then, the liquid reaction mixture was concentrated under reduced pressure to give the title compound as a white solid.

ESI-MS Found: m/z 179[M+H]⁺

### Referential Example 3-31

### Synthesis of tert-butyl 4-(1-methyl-2-oxo-4-5-dihydropyridin-4-yl)piperidine-1-carboxylate and tert-butyl 4-(2-methoxypyridin-4-yl)piperidine-1-carboxylate

To an N,N-dimethylformamide solution (50 ml) of the compound (1.39 g) obtained in Referential Example 3-29, methyl iodide (374 μl) and caesium fluoride (1.52 g) were added at 0° C. and the resultant mixture was stirred at room temperature for 22 hours. After adding water, the liquid reaction mixture was extracted with ethyl acetate. The organic layer was washed with a saturated aqueous solution of sodium chloride and dried over anhydrous sodium sulfate. Then, the organic layer was concentrated under reduced pressure and the residue was purified by silica gel column chromatography (hexane:ethyl acetate=95:5 to 0:100 followed by chloroform:methanol:95:5). Thus, tert-butyl 4-(1-methyl-2-oxo-1-2-dihydropyridin-4-yl)piperidine-1-carboxylate (1.65 g) was obtained as a yellow amorphous substance while tert-butyl 4-(2-methoxypyridin-4-yl)piperidine-1-carboxylate (0.12 g) was obtained as a yellow oily substance.

### tert-butyl 4-(1-methyl-2-oxo-1-2-dihydropyridin-4-yl)piperidine-1-carboxylate

ESI-MS Found: m/z 293[M+H]⁺

### tert-butyl 4-(2-methoxypyridin-4-yl)piperidine-1-carboxylate

ESI-MS Found: m/z 293[M+H]⁺

### Referential Example 3-32

### Synthesis of tert-butyl 4-[1-(difluoromethyl)-2-oxo-1,2-dihydropyridin-4-yl]piperidine-1-carboxylate

To an acetonitrile solution (12.5 ml) of the compound (0.50 g) obtained in Referential Example 3-28, sodium hydrogencarbonate (749 mg) and 2-(fluorosulfonyl)difluoroeacetic acid (736 pd) were added at room temperature and the resultant mixture was stirred at 45° C. for 4 hours. After adding a saturated aqueous sodium hydrogencarbonate solution, the liquid reaction mixture was extracted with ethyl acetate. The organic layer was washed with a saturated aqueous solution of sodium chloride and dried over anhydrous sodium sulfate. Then, the organic layer was concentrated under reduced pressure and the residue was purified by silica gel column chromatography (hexane:ethyl acetate=100:0 to 70:30). Thus, the title compound (443 mg) was obtained as a yellow oily substance.

ESI-MS Found: m/z 329[M+H]

### Referential Example 3-33

### Synthesis of 1-methyl-4-piperidin-4-ylpyridin-2(1H)-one hydrochloride

The procedure of Referential Example 3-11 was followed but using tert-butyl 4-(1-methyl-2-oxo-1-2-dihydropyridin-4-yl)piperidine-1-carboxylate (1.65 g) obtained in Referential Example 3-31 to give the title compound (930 mg) as a white solid.

ESI-MS Found: m/z 193 [M+H]⁺

### Referential Example 3-34

### Synthesis of 2-methoxy-4-piperidin-4-ylpyridine dihydrochloride

The procedure of Referential Example 3-11 was followed but using tert-butyl 4-(2-methoxypyridin-4-yl)piperidine-1-carboxylate (120 mg) obtained in Referential Example 3-31 to give the title compound (111 mg) as a white solid.

ESI-MS Found: m/z 193[M+H]⁺

### Referential Example 3-35

The following amines (V) were obtained by using the compounds 27 and alkylating agents (including literature-known compounds) and conducting reactions under the conditions as shown in Referential Examples 3-28 to 3-33.

TABLE 5-1
Synthesis of amine (V)

| Ar—X 27 | R—X | 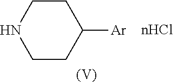 | ESI-MS |
|---|---|---|---|
| 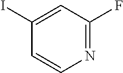 | none | 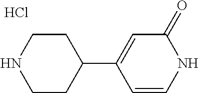 | 179 [M + H]⁺ |
| | CH₃—I | 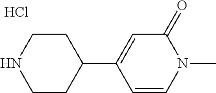 | 193 [M + H]⁺ |
| | | 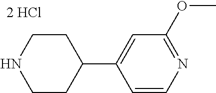 | 193 [M + H]⁺ |
| | 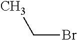 | 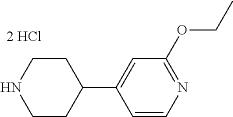 | 207 [M + H]⁺ |
| | FSO₂CF₂CO₂H | 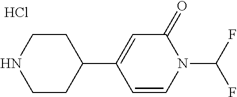 | 229 [M + H]⁺ |
| 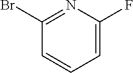 | none | 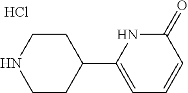 | 179 [M + H]⁺ |
| | CH₃—I | 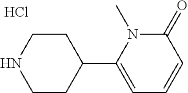 | 193 [M + H]⁺ |
| | | 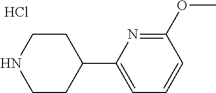 | 193 [M + H]⁺ |

TABLE 5-2
Synthesis of amine (V)

| Ar—X 27 | R—X | 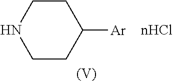 | ESI-MS |
|---|---|---|---|
| 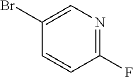 | none | 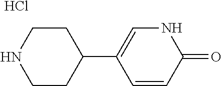 | 179 [M + H]⁺ |
| | CH₃—I | 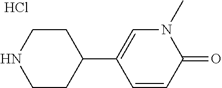 | 193 [M + H]⁺ |
| | 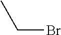 | 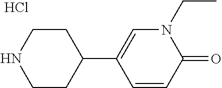 | 207 [M + H]⁺ |
| | | 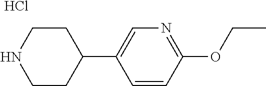 | 207 [M + H]⁺ |
| | FSO₂CF₂CO₂H | 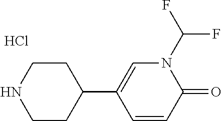 | 229 [M + H]⁺ |
| 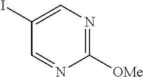 | none | 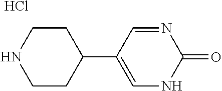 | — |
| | CH₃—I | 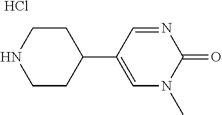 | — |

### Referential Example 3-36

### Synthesis of tert-butyl 4-pyridin-4-ylpiperidine-1-carboxylate

The procedures of Referential Examples 3-9 and 3-10 were followed but using 4-bromopyridine hydrochloride (925.4 mg) to give the title compound (1.77 g) as a yellow oily substance.

ESI-MS Found: m/z 263[M+H]⁺

### Referential Example 3-37

### Synthesis of tert-butyl 4-(1-oxidopyridine-4-yl)piperidine-1-carboxylate

To a chloroform solution (10 ml) of the compound (1.01 g) obtained in Referential Example 3-36, m-chlorobenzoic acid (1.38 g) was added at room temperature and the resultant mixture was stirred for 5 hours. After adding water, the liquid reaction mixture was extracted with ethyl acetate. The organic layer was washed with a saturated aqueous solution of sodium chloride and dried over anhydrous sodium sulfate. Then, the organic layer was concentrated under reduced pressure and the residue was purified by silica gel column chromatography (chloroform:methanol=100:0 to 90:10). Thus, the title compound (889.8 mg) was obtained as a yellow oily substance.

ESI-MS Found: m/z 279[M+H]⁺

### Referential Example 3-38

### Synthesis of 4-piperidin-4-ylpyridine-1-oxide hydrochloride

The procedure of Referential Example 3-11 was followed but using the compound (890 mg) obtained in Referential Example 3-37 to give the title compound (806 mg) as a white solid.

ESI-MS Found: m/z 179[M+H]⁺

### Referential Example 3-39

### Synthesis of 3-piperidin-4-ylpyridine-1-oxide hydrochloride

The procedures of Referential Examples 3-36 to 3-38 were followed but using 3-bromopyridine to give the title compound as a white solid.

ESI-MS Found: m/z 179[M+H]⁺

### Referential Example 4

### Synthesis of Compound Represented by Formula (II) (Ketone (II)

### Referential Example 4-1

### Synthesis of (3,4-difluorophenyl){4-[(6-fluoro-1H,1′H-spiro[flo[3,4-c]pyridin-3,4′-piperidin]-1′-yl)methylphenyl]methanone

To an ethyl acetate solution (13.0 ml) of the compound (1.00 g) obtained in Referential Example 1-8 and triethylamine (1.98 ml), chloromethanesulfonic acid (780 μl) was added at 0° C. and the resultant mixture was stirred for 20 minutes. After adding an aqueous sodium hydrogencarbonate solution, the liquid reaction mixture was extracted with ethyl acetate. The organic layer was washed with a saturated aqueous solution of sodium chloride and dried over anhydrous sodium sulfate. Then, the organic layer was concentrated under reduced pressure. Thus, a crude product (1.33 g) of 4-(3,4-difluorobenzoyl)benzylmethanesulfonate was obtained as a white solid.

To a chloroform solution (1.20 ml) of 2-fluoro-6-piperidin-4-ylpyrimidine hydrochloride (81.2 mg) obtained in Referential Example 3-14, triethylamine (126 μl) was added at 0° C. Next, the mesilate (100 mg) obtained above was added thereto and the resultant mixture was stirred at room temperature for 15 hours. After adding an aqueous sodium hydrogencarbonate solution, the liquid reaction mixture was extracted with chloroform. The organic layer was washed with a saturated aqueous solution of sodium chloride and dried over anhydrous sodium sulfate. Then, the organic layer was concentrated under reduced pressure and the residue was purified by silica gel column chromatography (chloroform:methanol 95:5). Thus, the title compound (103 mg) was obtained as an orange oily substance.

ESI-MS Found: m/z 411[M+H]⁺

### Referential Example 4-2

The procedure of Referential Example 4-1 was followed but using the compounds 10 or bromides (IV) obtained in Referential Examples 1 and 2 or various amines (V) obtained in Referential Example 3 to give the following ketones (II).

TABLE 9
Table 6: Synthesis of ketone (II)

| 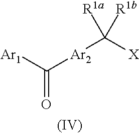 | 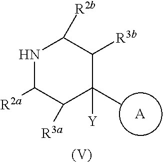 | 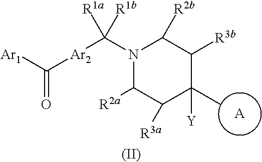 | ESI-MS |
|---|---|---|---|
| 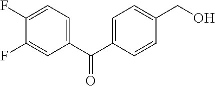 | 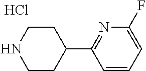 | 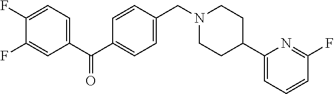 | 411 [M + H]⁺ |
| | 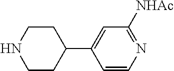 | 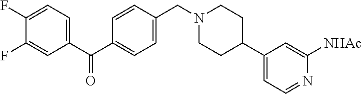 | 450 [M + H]⁺ |
| | 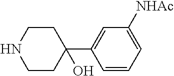 | 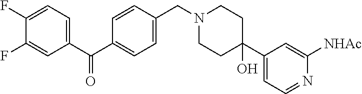 | 465 [M + H]⁺ |
| 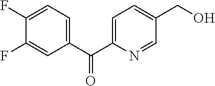 | 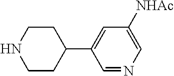 | 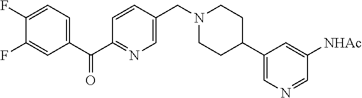 | 451 [M + H]⁺ |
| | 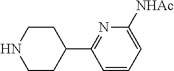 | 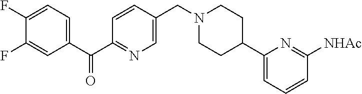 | 451 [M + H]⁺ |
| 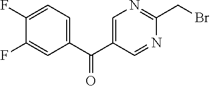 | 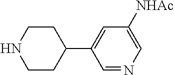 | 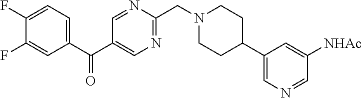 | 452 [M + H]⁺ |
| 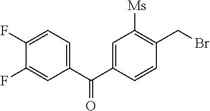 | 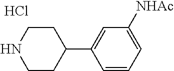 | 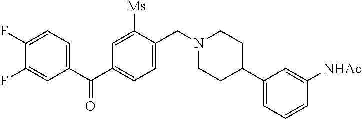 | — |
| 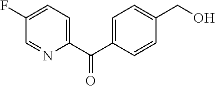 | 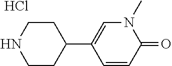 | 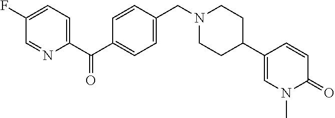 | 406 [M + H]⁺ |

### Referential Example 5

### Synthesis of Compound Represented by Formula (IVc″) (Oxime (IVc″))

### Referential Example 5-1

### Synthesis of (Z)-(3,4-difluorophenyl)[5-(hydroxymethyl)-2-pyridinyl]methanone o-(2-hydroxy-2-methylpropyl)oximde and (E)-(3,4-difluorophenyl)[5-(hydroxymethyl)-2-pyridinyl]methanone o-(2-hydroxy-2-methylpropyl)oximde

To a pyridine solution (25.0 ml) of (3,4-difluorophenyl)[5-(hydroxymethyl)-2-pyridinyl]methanone (1.36 g) obtained in Referential Example 1-9, O-(2-hydroxy-2-methylpropyl)hydroxylamine hydrochloride (2.41 g) was added and the resultant mixture was stirred at room temperature overnight. After distilling off pyridine under reduced pressure and adding an aqueous sodium hydrogencarbonate solution, the liquid reaction mixture was extracted with ethyl acetate. The organic layer was washed with a saturated aqueous solution of sodium chloride and dried over anhydrous sodium sulfate. Then, the organic layer was concentrated under reduced pressure and the residue was purified by silica gel column chromatography (hexane:ethyl acetate=100:0 to 0:100). After resolving geometric isomers by high performance liquid chromatography (YMC-CombiPrep™ proC-18, H₂O (0.1% TFA):CH₃CN (0.1% TFA)=90:10 to 50:50), the (Z)-isomer (435 mg) of the title compound was obtained as a white amorphous substance, while the (E)-isomer (1.17 g) of the title compound was obtained as a pale yellow amorphous substance.

### (Z)-Isomer of Title Compound

¹HNMR (400 MHz, CDCl₃, δ ppm): 1.21 (6H, s), 4.10 (2H, s), 4.83 (2H, s), 7.08-7.17 (2H, ni), 7.31-7.37 (1H, m), 7.50 (1H, d, J=8.3 Hz), 7.88 (1H, dd, J=8.3, 2.2 Hz), 8.72 (1H, d, J=1.5 Hz).

ESI-MS Found: m/z 337[M+H]⁺

### (E)-Isomer of Title Compound

¹ HNMR (400 MHz, CDCl₃, δ ppm): 1.25 (6H, s), 4.15 (2H, s), 4.77 (2H, s), 7.15-7.33 (3H, m), 7.78-7.79 (2H, m), 8.56 (1H, s).

ESI-MS Found: m/z 337[M+H]⁺

### Referential Example 5-2

The following oximes (IVc″) were obtained by using the compounds 10 or promides (IV) obtained in Referential Examples 1 and 2 and various hydroxylamine hydrochlorides (III) (including literature-known compounds) and conducting the procedure as shown in Referential Example 5-1.

TABLE 7-1
Synthesis of oxime (IVc″)

| 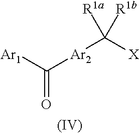 | H₂NZ.HCl (III) | 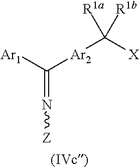 | ESI-MS |
|---|---|---|---|
| 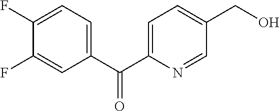 | 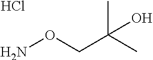 | 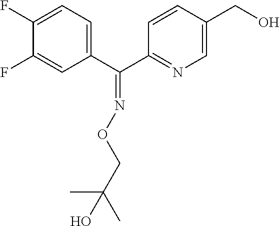 | (Z) 337 [M + H]⁺ (E) 337 [M + H]⁺ |
| | 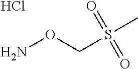 | 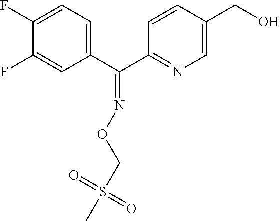 | (Z) 357 [M + H]⁺ (E) 357 [M + H]⁺ |
| | H₂NOH.HCl | 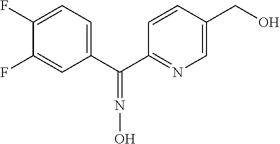 | 265 [M + H]⁺ |
| 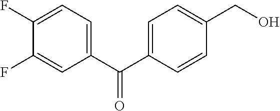 | 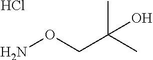 | 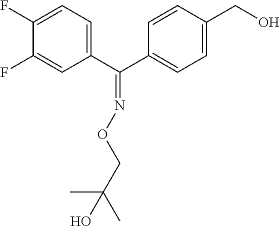 | (Z) 336 [M + H]⁺ (E) 336 [M + H]⁺ |
| | H₂NOH.HCl | 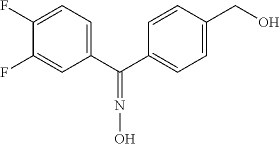 | 264 [M + H]⁺ |
| 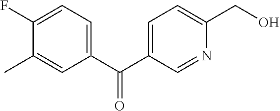 | H₂NOH.HCl | 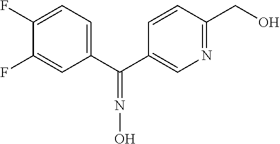 | (Z) 265 [M + H]⁺ (E) 265 [M + H]⁺ |

TABLE 7-2
Synthesis of oxime (IVc″)

| 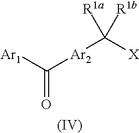 | H2NZ.HCl (III) | 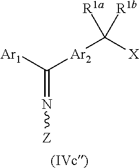 | ESI-MS |
|---|---|---|---|
| 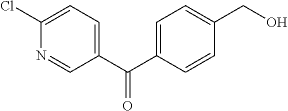 | 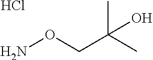 | 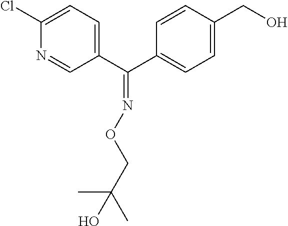 | 335 [M + H]⁺ |
| 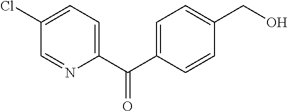 | 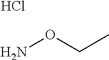 | 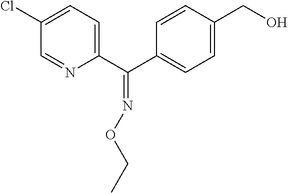 | 291 [M + H]⁺ |
| 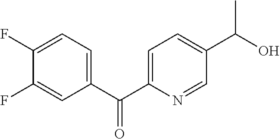 | 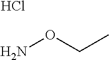 | 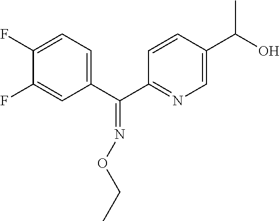 | 307 [M + H]⁺ |

### Referential Example 5-3

### Synthesis of [({(1Z)-(3,4-difluorophenyl)[5-(hydroxymethyl)pyridin-2-yl]methylene}amino)oxy]acetonitrile and [({(1E)-(3,4-difluorophenyl)[5-(hydroxymethyl)pyridin-2-yl]methylene}amino)oxy]acetonitrile

To a DMF solution (20.0 ml) of (3,4-difluorophenyl)[5-(hydroxymethyl)pyridin-2-yl]methanone oxime (2.15 g) obtained in Referential Example 5-2, potassium carbonate (2.25 g) and bromoacetonitrile (0.68 ml) were added at room temperature and the resultant mixture was stirred overnight. After adding an aqueous sodium hydrogencarbonate solution, the liquid reaction mixture was extracted with ethyl acetate. The organic layer was washed with water and a saturated aqueous solution of sodium chloride and dried over anhydrous sodium sulfate. Then, the organic layer was concentrated under reduced pressure and the residue was purified by preparative thin layer silica gel chromatography (chloroform:methanol=10:0 to 19:1). After resolving geometric isomers by high performance liquid chromatography (YMC-CombiPrep™ proC-18, H₂O (0.1% TFA):CH₃CN (0.1% TFA)=90:10 to 50:50), the (Z)-isomer (523 mg) of the title compound was obtained as a pale yellow solid, while the (E)-isomer (1.20 g) of the title compound was obtained as a colorless solid.

### (Z)-Isomer of Title Compound

ESI-MS Found: m/z 304[M+H]⁺

### (E)-Isomer of Title Compound

ESI-MS Found: m/z 304[M+H]⁺

### Referential Example 5-4

The following oximes (IVc″) were obtained by using various oximes (Z═N—OH: formula (IVc″)) obtained in Referential Example 5-2 and the compounds represented by formula (IIIa) (including literature-known compounds) and conducting the procedure as shown in Referential Example 5-3.

TABLE 8
Synthesis of oxime (IVc″)

| 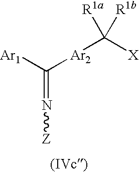 | H₂NZ•HCl (IIIa) | 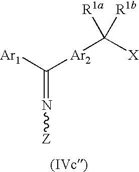 | ESI-MS |
|---|---|---|---|
| 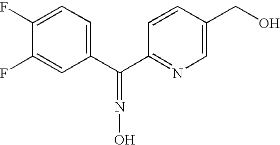 | 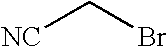 | 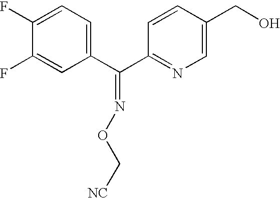 | (Z) 304 [M + H]⁺ (E) 304 [M + H]⁺ |
| | 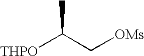 | 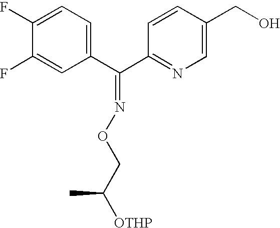 | 407 [M + H]⁺ |
| | 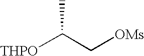 | 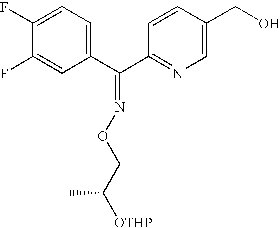 | 407 [M + H]⁺ |
| 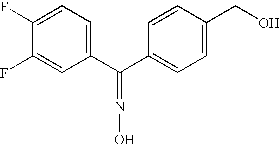 | 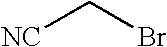 | 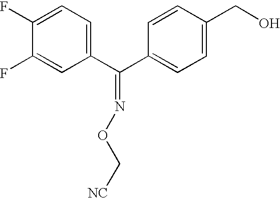 | 303 [M + H]⁺ |
| 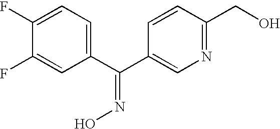 | 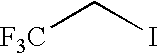 | 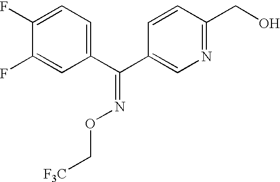 | — |

### Referential Example 6

### Synthesis of Compound Represented by Formula (IVc′) (Oxime (IVc′))

### Referential Example 6-1

### Synthesis of 4-[(acetyloxy)methyl]benzoic acid

To a chloroform solution (300 ml) of 4-(hydroxymethyl)benzoic acid (18.3 g), acetic anhydride (60.0 ml) and pyridine (200 ml) were added and the resultant mixture was stirred at 80° C. for 3 hours. After concentrating the liquid reaction mixture under reduced pressure, water (600 ml) was added to the residue followed by stirring at 90° C. for 5 hours. Then, the liquid reaction mixture was cooled to 0° C. and the white solid thus formed was collected by filtration. The obtained solid was washed with water and dissolved in ethyl acetate. The organic layer was washed with a saturated aqueous solution of sodium chloride and dried over anhydrous sodium sulfate. The organic layer was concentrated under reduced pressure. Then, the title compound (21.0 g) was obtained as a white solid.

ESI-MS Found: m/z 217[M+Na]⁺

### Referential Example 6-2

### Synthesis of 4-{[(2-fluoroethoxy)amino]carbonyl}benzyl acetate

To a DMF solution (160 ml) of the compound (7.77 g) obtained in Referential Example 6-1, O-(2-fluoroethyl)hydroxylamine hydrochloride (2.41 g), N-ethyldiisopropylamine (35.0 ml) and O-(7-azabenzotriazol-1-yl)-N,N,N′,N′-tetramethyluronium hexafluorophosphoric acid (16.7 g) were added at 0° C. and the resultant mixture was stirred at room temperature overnight. After adding water, the liquid reaction mixture was extracted with ethyl acetate. The organic layer was washed with a saturated aqueous solution of sodium chloride and dried over anhydrous sodium sulfate. The organic layer was concentrated under reduced pressure and the residue was purified by silica gel column chromatography (hexane:ethyl acetate=50:50 to 0:100). Thus, the title compound (7.39 g) was obtained as a white solid.

ESI-MS Found: m/z 256[M+H]⁺

### Referential Example 6-3

### Synthesis of 4-{(Z)-bromo[(2-fluoroethoxy)imino]methyl}benzyl acetate

To an acetonitrile solution (150 ml) of the compound (8.41 g) obtained in Referential Example 6-2, carbon tetrabromide (14.9 g) and triphenylphosphine (11.8 g) were added and the resultant mixture was stirred at 80° C. for 4 hours. After cooling the liquid reaction mixture to room temperature, the white solid thus formed was filtered off. The filtrate was concentrated under reduced pressure and the residue was purified by silica gel column chromatography (hexane:ethyl acetate-100:0 to 50:50). Thus, the title compound (8.76 g) was obtained as a yellow oily substance.

ESI-MS Found: m/z 318[M+H]⁺

### Referential Example 6-4

### Synthesis of 4-{(Z)-(3,4-difluorophenyl)[(2-fluoroethoxy)imino]methyl}benzyl acetate

To a toluene solution (116 ml) of the compound (9.26 g) obtained in Referential Example 6-3, 3,4-difluorophenylboric acid (5.51 g), palladium acetate (653 mg), triphenylphosphine (1.53 g) and a 2M aqueous sodium carbonate solution (29.0 ml) were added and the resultant mixture was stirred at 80° C. overnight. After adding an aqueous sodium hydrogencarbonate solution, the liquid reaction mixture was extracted with ethyl acetate. The organic layer was washed with a saturated aqueous solution of sodium chloride and dried over anhydrous sodium sulfate. The organic layer was concentrated under reduced pressure and the residue was purified by silica gel column chromatography (hexane:ethyl acetate=100:0 to 0:100). Thus, the title compound (8.97 g) was obtained as a yellow oily substance.

ESI-MS Found: m/z 352[M+H]⁺

### Referential Example 6-5

### Synthesis of (Z)-(3,4-difluorophenyl)[4-(hydroxymethyl)phenyl]methanone O-(2-fluoroethyl)oxime

To a solution of the compound (8.97 g) obtained in Referential Example 6-4 in a mixture of methanol (200 ml) with water (50.0 ml), potassium carbonate (6.91 g) was added and the resultant mixture was stirred at room temperature for 1 hour. After distilling off methanol, an aqueous ammonium chloride solution was added and the mixture was extracted with ethyl acetate. The organic layer was washed with a saturated aqueous solution of sodium chloride and dried over anhydrous sodium sulfate. The organic layer was concentrated under reduced pressure and the residue was purified by silica gel column chromatography (chloroform:ethyl acetate=100:0 to 70:30). Thus, the title compound (7.71 g) was obtained as a yellow oily substance.

ESI-MS Found: m/z 310[M+H]⁺

### Referential Example 6-6

### Synthesis of (Z)-[3,4-bis(methyloxy)phenyl][4-(hydroxymethyl)phenyl]methanone O-2-fluoroethyl)oxime

The procedures of Referential Examples 6-4 and 6-5 were followed but using the compound obtained in Referential Example 6-3 and 3,4-dimethoxyboronic acid. Thus, the title compound was obtained as a yellow oily substance.

ESI-MS Found: m/z 334[M+H]⁺

### Referential Example 6-7

### Synthesis of {5-[(Z)-bromo(ethoxyimino)methyl]pyridine-2-yl}methyl acetate

The procedures of Referential Examples 6-1 to 6-3 were followed but using 6-(hydroxymethyl)pyridine-3-carboxylic acid. Thus, the title compound was obtained as a pale yellow solid.

ESI-MS Found: m/z 301[M+H]⁺

### Referential Example 6-8

### Synthesis of (E)-(3,4-difluorophenyl)[6-hydroxymethyl)pyridine-3-yl]methanone O-ethyloxime

The procedures of Referential Examples 6-4 and 6-5 were followed but using the compound obtained in Referential Example 6-7. Thus, the title compound was obtained as a colorless oily substance.

ESI-MS Found: m/z 293[M÷H]⁺

### Referential Example 6-9

### Synthesis of {6-[(Z)-bromo(ethoxyimino)methyl]pyridine-3-yl]methyl acetate

The procedures of Referential Examples 6-1 to 6-3 were followed but using 5-(hydroxymethyl)pyridine-2-carboxylic acid. Thus, the title compound was obtained as a yellow oily substance.

ESI-MS Found: m/z 301[M+H]⁺

### Referential Example 6-10

### Synthesis of (E)-(3,4-difluorophenyl)[5-(hydroxymethyl)pyridine-2-yl]methanone O-ethyloxime

The procedures of Referential Examples 6-4 and 6-5 were followed but using the compound obtained in Referential Example 6-9. Thus, the title compound was obtained as a yellow oily substance.

ESI-MS Found: m/z 293[M+H]⁺

### Referential Example 7

### Synthesis of Compound Represented by Formula (IVa)

### Referential Example 7-1

### Synthesis of 5-bromo-N-methoxy-N-methylpyrimidine-2-carboxamide

The procedure of Referential Example 1-1 was followed but using 5-bromopyrimidine-2-carboxylic acid (21.6 g). Thus, the title compound (19.3 g) was obtained as a pale yellow solid.

ESI-MS Found: m/z 246[M+H]⁺

### Referential Example 7-2

### Synthesis of (5-bromopyrimidin-2-yl)(3,4-difluorophenyl)methanone

The procedure of Referential Example 2-2 was followed but using the compound (10.0 g) obtained in Referential Example 7-1. Thus, the title compound (11.7 g) was obtained as a pale yellow solid.

ESI-MS Found: m/z 299[M+H]⁺

### Referential Example 7-3

### Synthesis of 3,4-difluorophenyl)(5-vinylpyrimidin-2-yl)methanone

To an isopropyl alcohol solution (223 ml) of potassium vinyltrifluoroborate (7.19 g), [1,1′-bis(diphenylphosphino)ferrocene]palladium (II) dichloride (654 mg) and the compound (11.0 g) obtained in Referential Example 7-2, triethylamine (6.28 ml) was added and the resultant mixture was stirred at 80° C. for 2 hours. After concentrating the liquid reaction mixture under reduced pressure, the residue was purified by silica gel column chromatography (hexane:ethyl acetate=85:15 to 5:5). The solid thus formed was washed with a solvent mixture of diisropropyl ether/hexane (5:5). Thus, the title compound (7.80 g) was obtained as a pale brown solid.

ESI-MS Found: m/z 247[M+H]⁺

### Referential Example 7-4

### Synthesis of (3,4-difluorophenyl)[5-(1,2-dihydroxyethyl)pyrimidin-2-yl]methanone

To a solution of the compound (7.51 g) obtained in Referential Example 7-3 and 4-methylmorpholine-N-oxide (7.37 g) in a mixture of acetonitrile (90.0 ml) with water (30.0 ml), a 0.1 M aqueous osmium tetraoxide solution (3.05 ml) was added at room temperature and the resultant mixture was stirred overnight. After cooling to 0° C., an aqueous sodium thiosulfate solution was added to the liquid reaction mixture followed by extraction with ethyl acetate. The organic layer was washed with a 1N aqueous hydrochloric acid solution, an aqueous sodium hydrogencarbonate solution and a saturated aqueous solution of sodium chloride and then dried over anhydrous sodium sulfate. The organic layer was concentrated under reduced pressure and the residue was purified by silica gel column chromatography (chloroform:methanol=95:5 to 9:1). Thus, the title compound (6.62 g) was obtained as a brown solid.

ESI-MS Found: m/z 281[M+H]⁺

### Referential Example 7-5

### Synthesis of (Z)-(3,4-difluorophenyl)[5-(1,2-dihydroxyethyl)pyrimidin-2-yl)methanone O-cyclopropyloxime and (E)-(3,4-difluorophenyl)(5-(1,2-dihydroxyethyl)pyrimidin-2-yl)methanone O-cyclopropyloxime

The procedure of Referential Example 5-1 was followed but using the compound (199 mg) obtained in Referential Example 7-4 and O-cyclopropylhydroxylamine hydrochloride. Thus, the (Z)-isomer (63.9 mg) of the title compound was obtained as a pale brown oily substance, while the (E)-isomer (118 mg) of the title compound was obtained as a pale brown oily substance.

### (Z)-Isomer of Title Compound

ESI-MS Found: m/z 336[M+H]⁺

### (E)-Isomer of Title Compound

ESI-MS Found: m/z 336[M+H]⁺

### Referential Example 7-6

### Synthesis of 2-[(E)-[(cyclopropyloxy)imino](3,4-difluorophenyl)methyl]pyrimidine-5-carbaldehyde

To a THF solution (3.00 ml) of the (E)-compound (118 mg) obtained in Referential Example 7-5, an aqueous solution (3.00 ml) of sodium periodate (113 mg) was added at 0° C. and the resultant mixture was stirred at room temperature overnight. After cooling to 0° C., an aqueous ammonium chloride solution was added to the liquid reaction mixture followed by extraction with ethyl acetate. Thus, the title compound (106 mg) was obtained as a white amorphous substance.

ESI-MS Found: m/z 304[M+H]⁺

### Example 1-1

### Synthesis of N-{5-[1-({6-[(Z)-(3,4-difluorophenyl)(hydroxyimino)methyl]pyridin-3-yl}methyl)piperidin-4-yl]pyridine-3-yl}acetamide and N-{5-[1-({6-[(E)-(3,4-difluorophenyl)(hydroxyimino)methyl]pyridin-3-yl}methyl)piperidin-4-yl]pyridine-3-yl}acetamide

To a pyridine solution (11.0 ml) of N-[5-(1-{[6-(3,4-difluorobenzoyl)pyridin-3-yl]methyl)piperidin-4-yl)pyridine-3-yl}acetamide (500 mg) obtained in Referential Example 4-2, hydroxylamine hydrochloride (385 mg) was added at 0° C. and the resultant mixture was stirred at room temperature overnight. After adding an aqueous sodium hydrogencarbonate solution, the liquid reaction mixture was extracted with diethyl ether. The organic layer was washed with water and a saturated aqueous solution of sodium chloride and then dried over anhydrous sodium sulfate. The organic layer was concentrated under reduced pressure and the residue was purified by preparative thin layer silica gel chromatography (chloroform:methanol=100:0 to 80:20). By resolving geometric isomers by CHIRALPAK AD-H (hexaneisopropanol:diethylamine=75:25:0.025), the (Z)-isomer (100 mg, eluted earlier) of the title compound was obtained as a white solid, while the (E)-isomer (331 mg, eluted later) of the title compound was obtained as a white solid.

### (Z)-Isomer of Title Compound

¹HNMR (400 MHz, CD₃ OD, δ ppm): 1.80-1.93 (4H, m), 2.18 (3H, s), 2.25-2.33 (2H, m), 2.65-2.74 (1H, m), 3.08-3.14 (2H, m), 3.72 (2H, s), 7.14-7.18 (1H, m), 7.22-7.29 (1H, m), 7.43 (1H, ddd, J=11.9, 7.5, 2.1 Hz), 7.63 (1H, d, J=8.0 Hz), 8.01-8.03 (2H, m), 8.20 (1H, d, J=1.8 Hz), 8.60 (1H, d, J=2.3 Hz), 8.66 (1H, d, J=1.8 Hz).

ESI-MS Found: m/z 466[M+H]⁺

### (E)-Isomer of Title Compound

¹HNMR (400 MHz, CD₃ OD, δ ppm): 1.75-1.89 (4H, m), 2.15 (3H, s), 2.18-2.26 (2H, m), 2.59-2.68 (1H, m), 3.00-3.06 (2H, m), 3.63 (2H, s), 7.13-7.18 (1H, m), 7.28-7.40 (2H, m), 7.81-7.88 (2H, m), 7.98-8.00 (1H, m), 8.15 (1H, d, J=1.8 Hz), 8.44-8.46 (1H, m), 8.56 (1H, d, J=2.3 Hz).

ESI-MS Found: m/z 466[M+H]⁺

By using various ketones obtained in Referential Example 4 and hydroxylamine hydrochloride (including literature-known compounds), the procedure of Example 1-1 was followed to give the compounds of Examples 1-2 to 1-8.

### Example 1-2

### N-{5-[1-({6-[(Z)-(3,4-Difluorophenyl)(methoxyimino)methyl]pyridin-3-yl}methyl)piperidin-4-yl]pyridine-3-yl}acetamide

¹HNMR (400 MHz, CDCl₃, δ ppm): 1.80-1.85 (4H, m), 2.14-2.20 (5H, m), 2.55-2.61 (1H, m), 3.01-3.04 (2H, m), 3.60 (2H, s), 3.98 (3H, s), 7.07-7.18 (2H, m), 7.36-7.41 (1H, m), 7.52 (1H, d, J=8.0 Hz), 7.75 (1H, s), 7.84-7.86 (1H, m), 8.09 (1H, s), 8.23 (1H, s), 8.36 (1H, d, J=2.0 Hz), 8.65 (1H, s).

ESI-MS Found: m/z 480[M+H]⁺

### N-{5-[1-({6-[(E)-(3,4-Difluorophenyl)(methoxyimino)methyl]pyridin-3-yl}methyl)piperidin-4-yl]pyridine-3-yl}acetamide

¹ HNMR (400 MHz, CDCl₃, δ ppm): 1.74-1.82 (4H, m), 2.09-2.18 (5H, m), 2.51-2.57 (1H, m) 2.95-2.98 (2H, m), 3.56 (2H, s), 4.04 (3H, s), 7.11-7.32 (3H, m), 7.76 (2H, s), 8.06 (1H, s), 8.19 (1H, s), 8.39 (1H, s), 8.45 (1H, s), 8.51 (1H, s).

ESI-MS Found: m/z 480[M+H]⁺

### Example 1-3

### N-{5-[1-({5-[(E)-(3,4-Difluorophenyl)(ethoxyimino)methyl]pyrimidin-2-yl}methyl)piperidin-4-yl]pyridine-3-yl}acetamide

¹HNMR (400 MHz, CDCl₃, δ ppm): 1.33 (3H, t, J=7.0 Hz), 1.81-1.99 (4H, m), 2.20 (3H, s), 2.28 (2H, td, J=11.7, 2.3 Hz), 2.54-2.62 (1H, m), 3.11-3.14 (2H, m), 3.89 (2H, s), 4.30 (2H, q, J=7.0 Hz), 7.08-7.12 (1H, m), 7.23-7.36 (2H, m), 7.67 (1H, s), 8.02-8.04 (1H, m), 8.23 (1H, s), 8.41 (1H, d, J=2.2 Hz), 8.80 (2H, s).

ESI-MS Found: m/z 495[M+H]⁺

### N-{5-[1-({5-[(Z)-(3,4-Difluorophenyl)(ethoxyimino)methyl]pyrimidin-2-yl}methyl)piperidin-4-yl]pyridine-3-yl}acetamide

¹HNMR (400 MHz, CDCl₃, δ ppm): 1.31 (3H, t, J=7.0 Hz), 1.84-1.87 (2H, m), 1.93-2.03 (2H, m), 2.20 (3H, s), 2.29-2.35 (2H, m), 2.57-2.65 (1H, m), 3.18-3.20 (2H, m), 3.92 (2H, s), 4.28 (2H, q, J=7.1 Hz), 7.10-7.20 (2H, m), 7.44 (1H, ddd, J=11.3, 7.6, 2.0 Hz), 7.96 (1H, s), 8.08 (1H, s), 8.23 (1H, s), 8.41 (1H, d, J=2.0 Hz), 8.77 (2H, s).

ESI-MS Found: m/z 495[M+H]⁺

### Example 1-4

### N-{6-[1-({6-[(E)-(3,4-Difluorophenyl)(ethoxylmino)methyl]pyridin-3-yl}methyl)piperidin-4-yl]pyridine-2-yl}acetamide

¹HNMR (400 MHz, CDCl₃, δ ppm): 1.33 (3H, t, J=7.0 Hz), 1.78-1.89 (4H, m), 2.08-2.16 (2H, m), 2.19 (3H, s), 2.51-2.60 (1H, m), 2.94-3.00 (2H, m), 3.56 (2H, s), 4.30 (2H, q, J=7.0 Hz), 6.90 (1H, d, J=7.6 Hz), 7.13-7.24 (2H, m), 7.29-7.34 (1H, m), 7.62 (1H, t, J=7.9 Hz), 7.74-7.80 (2H, m), 7.88 (1H, s), 7.99 (1H, d, J=8.0 Hz), 8.51 (1H, s).

ESI-MS Found: m/z 494[M+H]⁺

### N-{6-[1-({6-[(Z)-(3,4-Difluorophenyl)(ethoxyimino)methyl]pyridin-3-yl}methyl)piperidin-4-yl]pyridine-2-yl}acetamide

¹ HNMR (400 MHz, CDCl₃, δ ppm): 1.31 (3H, t, J=7.0 Hz), 1.81-1.94 (4H, m), 2.16-2.21 (5H, m), 2.56-2.65 (1H, m), 3.02-3.08 (2H, m), 3.62 (2H, s), 4.26 (2H, q, J=7.0 Hz), 6.93 (1H, d, J=7.6 Hz), 7.07-7.21 (2H, m), 7.35-7.41 (1H, m), 7.57 (1H, d, 8.0 Hz), 7.64 (1H, t, J=7.9 Hz), 7.83-7.87 (2H, m), 8.01 (1H, d, J=8.4 Hz), 8.67 (1H, s).

ESI-MS Found: m/z 494[M+H]⁺

### Example 1-5

### N-{4-[1-(4-{(E)-(3,4-Difluorophenyl)[2-fluoroethoxy)imino]methyl}benzyl)piperidin-4-yl]pyridine-2-yl}acetamide

¹HNMR (400 MHz, CDCl₃, δ ppm): 1.81-1.86 (4H, m), 2.10-2.16 (2H, m), 2.19 (3H, s), 2.55-2.57 (1H, m), 3.04-3.07 (2H, m), 3.58 (2H, s), 4.36-4.45 (2H, m), 4.62-4.76 (2H, m), 6.93-6.94 (1H, m), 7.10-7.18 (2H, m), 7.32-7.44 (5H, m), 8.14-8.17 (2H, m), 9.01 (1H, s).

ESI-MS Found: m/z 511[M+H]⁺

### N-{4-[1-(4-{(Z)-(3,4-Difluorophenyl)[(2-fluoroethoxy)imino]methyl}benzyl)piperidin-4-yl]pyridine-2-yl}acetamide

¹HNMR (400 MHz, CDCl₃, δ ppm): 1.77-1.83 (4H, m), 2.05-2.12 (2H, m), 2.19 (3H, s), 2.50-2.58 (1H, m), 2.98-3.01 (2H, m), 3.55 (2H, s), 4.37-4.46 (2H, m), 4.62-4.76 (2H, m), 6.91-6.92 (1H, m), 7.11-7.15 (1H, m), 7.19-7.35 (4H, m), 7.40-7.42 (2H, m), 8.13-8.16 (2H, m), 8.93 (1H, s).

ESI-MS Found: m/z 511[M+H]⁺

### Example 1-6

### N-{3-[1-(4-{(Z)-(3,4-Difluorophenyl)[(2-fluoroethoxy)imino]methyl}benzyl)-4-hydroxypiperidin-4-yl]phenyl}acetamide

¹ HNMR (400 MHz, CDCl₃, δ ppm): 1.68-1.71 (2H, m), 2.03-2.14 (5H, m), 2.44-2.49 (2H, m), 2.73-2.76 (2H, m), 3.57 (2H, s), 4.37-4.46 (2H, m), 4.62-4.76 (2H, m), 7.11-7.14 (1H, m), 7.18-7.47 (10H, m), 7.60 (1H, s).

ESI-MS Found: m/z 526[M+H]⁺

### Example 1-7

### N-(3-{1-[4-{(Z)-(3,4-Difluorophenyl)[(2-fluoroethoxy)imino]methyl}-2-(methylsulfonyl)benzyl)piperidin-4-yl}phenyl)acetamide

¹HNMR (400 MHz, CDCl₃, δ ppm): 1.59-1.66 (4H, m), 1.82-1.85 (2H, m), 2.13 (3H, s), 2.17-2.22 (2H, m), 2.50-2.53 (1H, m), 3.04-3.07 (2H, m), 3.45 (3H, s), 3.96 (2H, s), 4.36-4.43 (2H, m), 4.59-4.71 (2H, m), 6.92-6.94 (1H, m), 7.07-7.46 (6H, m), 7.55 (1H, s), 8.06 (1H, s).

ESI-MS Found: m/z 588[M+H]⁺

### N-(3-{1-[4-{(E)-(3,4-Difluorophenyl)[(2-fluoroethoxy)imino]methyl}-2-(methylsulfonyl)benzyl)piperidin-4-yl}phenyl)acetamide

¹HNMR (400 MHz, CDCl₃, δ ppm): 1.58-1.62 (4H, m), 1.79-1.82 (2H, m), 2.12 (3H, s), 2.12-2.18 (2H, m), 2.47-2.53 (1H, m), 2.99-3.01 (2H, m), 3.45 (3H, s), 3.92 (2H, s), 4.37-4.46 (2H, m), 4.59-4.72 (2H, m), 6.88-6.90 (1H, m), 7.06-7.07 (1H, m), 7.20-7.27 (3H, m), 7.39-7.42 (2H, m), 7.53 (1H, d, J=7.8 Hz), 8.25 (1H, s).

ESI-MS Found: m/z 588[M+H]⁺

### Example 1-8

### 5-(1-{4-[(Z)-(Ethoxyimino)(5-fluoropyridin-2-yl)methyl]benzyl)piperidin-4-yl)-1-methylpyridin-2(1H)-one

¹ HNMR (400 MHz, CDCl₃, δ ppm): 1.29 (3H, t, J=7.0 Hz), 1.50-1.75 (4H, m), 1.95-2.10 (2H, m), 2.20-2.30 (1H, m), 2.93-3.02 (2H, m), 3.52 (3H, s), 3.53 (2H, s), 4.24 (2H, q, J=7.0 Hz), 6.54 (1H, d, J=9.2 Hz), 7.05 (1H, d, J=2.4 Hz), 7.24-7.32 (3H, m), 7.38-7.42 (2H, m), 7.51 (1H, dt, J=8.4, 3.0 Hz) 7.62 (1H, dd, J=8.4, 4.0 Hz), 8.57 (1H, d, J=3.0 Hz).

ESI-MS Found: m/z 449.3 [M+H]⁺

### 5-(1-{4-[(E)-(Ethoxylmino)(5-fluoropyridin-2-yl)methyl]benzyl)piperidin-4-yl)-1-methylpyridin-2(1H)-one

¹HNMR (400 MHz, CDCl₃, δ ppm): 1.34 (3H, t, J=7.0 Hz), 1.60-1.80 (4H, m), 2.00-2.13 (2H, m), 2.20-2.35 (1H, m), 3.00-3.08 (2H, m), 3.52 (3H, s), 3.56 (2H, s), 4.29 (2H, q, J=7.0 Hz), 6.55 (1H, d, J=9.2 Hz), 7.07 (1H, d, J=2.8 Hz), 7.26-7.32 (2H, m), 7.35-7.45 (4H, m), 7.81 (1H, dd, J=8.8, 4.4 Hz), 8.43 (1H, d, J=2.8 Hz).

ESI-MS Found: m/z 449.3 [M+H]+

### Example 2-1

### Synthesis of N-{5-[1-({6-[(E)-(3,4-difluorophenyl)(propoxyimino)methyl]pyridin-3-yl}methyl)piperidin-4-yl]pyridine-3-yl}acetamide

To a DMF solution (500 μl) of N-{5-[1-({6-[(E)-(3,4-difluorophenyl)(hydroxyimino)methyl]pyridin-3-yl}methyl)piperidin-4-yl]pyridine-3-yl}acetamide (23 mg) obtained in Example 1-1, potassium carbonate (21 mg) and 1-bromopropane (9.1 μl) were added at 0° C. and the resultant mixture was stirred at room temperature overnight. After adding an aqueous sodium hydrogencarbonate solution, the liquid reaction mixture was extracted with diethyl ether. The organic layer was washed with water and a saturated aqueous solution of sodium chloride and then dried over anhydrous sodium sulfate. The organic layer was concentrated under reduced pressure and the residue was purified by silica gel column chromatography (chloroform:methanol=100:0 to 90:10). Thus, the title compound (24.4 mg) was obtained as a white solid.

¹HNMR (400 MHz, CDCl₃, δ ppm): 0.95 (3H, t, J=7.4 Hz), 1.70-1.83 (6H, m), 2.11-2.16 (5H, m), 2.51-2.59 (1H, m), 2.96-2.99 (2H, m), 3.57 (2H, s), 4.21 (2H, t, J=6.7 Hz), 7.13-7.24 (2H, m), 7.28-7.34 (1H, m), 7.75-7.81 (2H, m), 8.05-8.07 (1H, m), 8.21 (1H, s), 8.25 (1H, s), 8.39 (1H, d, J=2.2 Hz), 8.51 (1H, s).

ESI-MS Found: m/z 508[M+H]⁺

By using various ketones obtained in Referential Example 4 and alkylating agents, the procedures of Examples 1-1 and 2-1 were followed to give the compounds of Examples 2-2 to 2-5

### Example 2-2

### N-{5-[1-({6-[(E)-3,4-Difluorophenyl)(isopropoxyimino)methyl]pyridin-3-yl}methyl)piperidin-4-yl]pyridine-3-yl}acetamide

¹ HNMR (400 MHz, CDCl₃, δ ppm): 1.31 (6H, d, J=6.3 Hz), 1.72-1.84 (4H, m), 2.09-2.18 (5H, m), 2.51-2.59 (1H, m), 2.97-3.00 (2H, m), 3.57 (2H, s), 4.50-4.59 (1H, m), 7.13-7.22 (2H, m), 7.30-7.35 (1H, m), 7.76 (1H, dd, J=8.1, 2.1 Hz), 7.82-7.84 (1H, m), 8.05-8.06 (1H, m), 8.21 (1H, d, J=1.6 Hz), 8.26 (1H, s), 8.39 (1H, d, 2.2 Hz), 8.50 (1H, d, J=1.6 Hz).

ESI-MS Found: m/z 508[M+H]⁺

### Example 2-3

### N-{5-[1-({6-[(E)-(2,2-Difluoroethoxy)imino](3,4-difluorophenyl)methyl]pyridin-3-yl}methyl)piperidin-4-yl]pyridine-3-yl}acetamide

¹HNMR (400 MHz, CDCl₃, δ ppm): 1.72-1.85 (4H, m), 2.10-2.19 (5H, m), 2.51-2.59 (1H, m), 2.96-2.99 (2H, m), 3.57 (2H, s), 4.39 (2H, td, J=13.3, 4.1 Hz), 6.07 (1H, tt, J=55.4, 4.2 Hz), 7.13-7.31 (3H, m), 7.76-7.79 (2H, m), 8.08-8.10 (2H, m), 8.21 (1H, s), 8.36 (1H, s), 8.53 (1H, s).

ESI-MS Found: m/z 530[M+H]⁺

### Example 2-4

### 2-({[(1E)-[5-({4-[5-(Acetylamino)pyridin-3-yl]piperidin-1-yl}methyl)pyridin-2-yl] (3,4-difluorophenyl)methylene]amino}oxy)-N,N-dimethylacetamide

¹HNMR (400 MHz, CDCl₃, δ ppm): 1.71-1.84 (4H, m), 2.06-2.21 (5H, m), 2.49-2.58 (1H, m), 2.93-3.01 (8H, m), 3.56 (2H, s), 4.92 (2H, s), 7.16-7.24 (1H, m), 7.28-7.33 (1H, m), 7.45-7.51 (1H, m), 7.69-7.74 (2H, m), 8.04-8.06 (1H, m), 8.19 (1H, s), 8.42 (1H, d, J=2.2 Hz), 8.51 (2H, s).

ESI-MS Found: m/z 551[M+H]⁺

### Example 2-5

### N-[5-(1-{6-((Z)-(3,4-Difluorophenyl){[2-(dimethylamino)ethoxy]imino}methyl)pyridin-3-yl]methyl}piperidin-4-yl]pyridine-3-yl}acetamide

¹HNMR (400 MHz, CDCl₃, δ ppm): 1.78-1.84 (4H, m), 2.10-2.19 (5H, m), 2.25 (6H, s), 2.53-2.61 (1H, m), 2.66 (2H, t, J=5.9 Hz), 3.00-3.03 (2H, m), 3.60 (2H, s), 4.31 (2H, t, J=5.9 Hz), 7.07-7.19 (2H, m), 7.36-7.41 (1H, m), 7.55 (1H, d, J=8.0 Hz), 7.83-7.85 (1H, m), 8.08 (1H, s), 8.22 (1H, s), 8.30 (1H, s), 8.40 (1H, d, J=2.2 Hz), 8.65 (1H, s).

ESI-MS Found: m/z 537[M+H]⁺

### Example 3-1

### Synthesis of 4-[1-({6-[(E)-(3,4-difluorophenyl)(ethoxyimino)methyl]pyridin-3-yl}methyl)piperidin-4-yl]-1-methylpyridin-2(1H)one

The procedure of Referential Example 4-1 was followed but using (E)-(3,4-difluorophenyl)[5-(hydroxymethyl)pyridine-2-yl]methanone O-ethyloxime (470 mg) obtained in Referential Example 6-10. Thus, a crude product (596 mg) of the corresponding mesylate was obtained as a yellow oily substance. To a DMF solution (6.0 ml) of 1-methyl-4-piperidin-4-ylpyridin-2(1H)-one hydrochloride (411 mg) obtained in Referential Example 3-33, potassium carbonate (620 mg) was added at 0° C. Next, the mesylate (596 mg) obtained above was added thereto and the resultant mixture was stirred at room temperature overnight. After adding an aqueous sodium hydrogencarbonate solution, the liquid reaction mixture was extracted with chloroform. The organic layer was washed with a saturated aqueous solution of sodium chloride and then dried over anhydrous sodium sulfate. The organic layer was concentrated under reduced pressure and the residue was purified by silica gel column chromatography (chloroform:methanol=100:0 to 95:5). Thus, the title compound (903 mg) was obtained as a yellow oily substance.

¹HNMR (400 MHz, CDCl₃, δ ppm): 1.33 (3H, t, J=7.0 Hz), 1.61-1.82 (4H, m), 2.05-2.14 (2H, m), 2.27-2.37 (1H, m), 2.93-3.00 (2H, m), 3.51 (3H, s), 3.56 (2H, s), 4.30 (2H, q, J=7.0 Hz), 6.06 (1H, dd, J=6.8, 2.0 Hz), 6.41 (1H, d, J=2.0 Hz), 7.12-7.34 (4H, m), 7.72-7.81 (2H, m), 8.49 (1H, d, J=1.5 Hz).

ESI-MS Found: m/z 467[M+H]⁺

By using various oxime alcohols (IVc″) obtained in Referential Examples 5 and 6 and various amine precursors or amines (V) obtained in Referential Example 3 (including literature-known compounds), the procedure of Example 3-1 (including the procedures of Referential Example 3-11 in the case of using an amine precursor) was followed to give the compounds of Examples 3-2 to 3-80.

### Example 3-2

### (E)-(3,4-Difluorophenyl)(5-{[4-6-fluoropyridin-3-yl)-4-hydroxypiperidin-1-yl]methyl}pyridin-2-yl)methanone O-ethyloxime

¹HNMR (400 MHz, CDCl₃, δ ppm): 1.33 (3H, t, J=7.0 Hz), 1.70-1.80 (2H, m), 2.05-2.15 (2H, m), 2.45-2.55 (2H, m), 2.75-2.80 (2H, m), 3.60 (2H, s), 4.30 (2H, q, J=7.0 Hz), 6.91 (1H, dd, J=8.3, 2.9 Hz), 7.13-7.24 (2H, m), 7.31 (1H, ddd, J=10.4, 7.6, 4.2 Hz), 7.74 (1H, dd, J=8.3, 2.0 Hz), 7.80 (1H, d, J=8.3 Hz), 7.90-7.95 (1H, m), 8.33 (1H, d, J=2.4 Hz), 8.52 (1H, d, J=1.5 Hz).

ESI-MS Found: m/z 471[M+H]⁺

### Example 3-3

### (E)-(3,4-Difluorophenyl){5-[(4-phenylpiperidin-1-yl)methyl]pyridin-2-yl)methanone O-ethyloxime

¹ HNMR (400 MHz, CDCl₃, δ ppm): 1.33 (3H, t, J=6.8 Hz), 1.70-1.90 (4H, m), 2.08-2.18 (2H, m), 2.45-2.55 (1H, m), 2.95 (2H, m), 3.57 (2H, s), 4.30 (2H, q, J=6.8 Hz), 7.12-7.25 (5H, m), 7.28-7.35 (3H, m), 7.73-7.80 (1H, m), 7.79 (1H, d, J=8.4 Hz), 8.50-8.57 (1H, m).

ESI-MS Found: m/z 436[M+H]⁺

### Example 3-4

### (E)-(3,4-Difluorophenyl) {5-{[4-(2-methoxypyridin-4-yl)piperidin-1-yl)methyl]pyridin-2-yl)methanone O-ethyloxime

¹HNMR (400 MHz, CDCl₃, δ ppm): 1.33 (3H, t, J=7.1 Hz), 1.68-1.85 (4H, m), 2.07-2.16 (2H, m), 2.41-2.51 (1H, m), 2.95-3.01 (2H, m), 3.57 (2H, s), 3.92 (3H, s), 4.30 (2H, q, J=7.0 Hz), 6.59 (1H, s), 6.74 (1H, dd, J=5.4, 1.5 Hz), 7.13-7.35 (3H, m), 7.74-7.82 (2H, m), 8.06 (1H, d, J=5.4 Hz), 8.51 (1H, d, J=1.5 Hz).

ESI-MS Found: m/z 467[M+H]⁺

### Example 3-5

### (E)-(3,4-Difluorophenyl)(5-{[4-(2-ethoxypyridin-4-yl)piperidin-1-yl]methyl}pyridin-2-yl)methanone O-ethyloxime

¹HNMR (400 MHz, CDCl₃, δ ppm): 1.33 (3H, t, J=7.1 Hz), 1.38 (3H, t, J=7.1 Hz), 1.69-1.85 (4H, m), 2.08-2.16 (2H, m), 2.40-2.50 (1H, m), 2.95-3.01 (2H, m), 3.57 (2H, s), 4.27-4.36 (4H, m), 6.57 (1H, s), 6.72 (1H, dd, J=5.4, 1.5 Hz), 7.12-7.35 (3H, m), 7.74-7.82 (2H, m), 8.04 (1H, d, J=5.4 Hz), 8.51 (1H, s).

ESI-MS Found: m/z 481[M+H]⁺

### Example 3-6

### 1-(Difluoromethyl)-4-[1-({6-[(E)-(3,4-difluorophenyl)(ethoxyimino)methyl]pyridin-3-yl}methyl)piperidin-4-yl]pyridin-2(1H)-one

¹HNMR (400 MHz, CDCl₃, δ ppm): 1.33 (3H, t, J=7.1 Hz), 1.60-1.85 (4H, m), 2.05-2.14 (2H, m), 2.29-2.39 (1H, m), 2.95-3.01 (2H, m), 3.56 (2H, s), 4.30 (2H, q, J=7.0 Hz), 6.20 (1H, dd, J=7.3, 2.0 Hz), 6.36 (1H, s), 7.13-7.81 (7H, m), 8.49 (1H, d, J=2.0 Hz).

ESI-MS Found: m/z 503[M+H]⁺

### Example 3-7

### 5-[1-({6-(E)-(3,4-Difluorophenyl)(ethoxyimino)methyl]piperidin-3-yl}methyl)piperidin-4-yl]pyridin-2(1H)-one

¹HNMR (400 MHz, CDCl₃, δ ppm): 1.33 (3H, t, J=7.0 Hz), 1.55-1.69 (2H, m), 1.72-1.80 (2H, m), 2.05-2.15 (2H, m), 2.24-2.34 (1H, m), 2.92-3.01 (2H, m), 3.56 (2H, s), 4.30 (2H, q, J=7.0 Hz), 6.56 (1H, d, J=9.3 Hz), 7.15-7.42 (5H, m), 7.71-7.82 (2H, m), 8.49 (1H, s).

ESI-MS Found: m/z 453 [M+H]⁺

### Example 3-8

### 5-[1-({6-[(E)-(3,4-Difluorophenyl)(ethoxyimino)methyl]pyridin-3-yl}methyl)piperidin-4-yl]-1-methylpyrimidin-2(1H)-one dihydrochloride

¹HNMR (400 MHz, DMSO-d₆, δ ppm): 1.36 (3H, t, J=7.1 Hz), 1.97-1.99 (2H, m), 2.12-2.23 (4H, m), 2.96-3.12 (1H, m), 3.63-3.72 (2H, m), 3.77 (3H, s), 4.37-4.46 (2H, m), 4.51-4.69 (2H, m), 7.25-7.33 (1H, m), 7.38-7.51 (2H, m), 7.95-8.03 (1H, m), 8.53-8.66 (1H, m), 8.80 (1H, s), 8.98-9.12 (2H, m).

ESI-MS Found: m/z 468[M+H]⁺

### Example 3-9

### 5-1-({6-[(E)-(3,4-Difluorophenyl)(ethoxylmino)methyl]pyridin-3-yl}methyl)piperidin-4-yl]-1-methylpyridin-2(1H)-one dihydrochloride

¹HNMR (400 MHz, DMSO-d₆, δ ppm): 1.35 (3H, t, J=7.3 Hz), 1.91-2.18 (8H, m), 2.79-2.94 (1H, m), 3.69 (3H, s), 4.41 (2H, q, J=7.3 Hz), 4.58 (2H, s), 6.77 (1H, d, J=9.3 Hz), 7.22-7.30 (1H, m), 7.36-7.49 (2H, m), 7.60-7.89 (2H, m), 7.97-8.07 (1H, m), 8.47-8.52 (1H, m), 8.97 (1H, s).

ESI-MS Found: m/z 467[M+H]⁺

### Example 3-10

### (E)-(3,4-Difluorophenyl){5-[(4-pyridin-4-ylpiperidin-1-yl)methyl]pyridin-2-yl}methanone O-ethyloxime

¹HNMR (400 MHz, CDCl₃, δ ppm): 1.33 (3H, t, J=7.0 Hz), 1.76-1.89 (4H, m), 2.08-2.25 (2H, m), 2.46-2.57 (1H, m), 2.97-3.07 (2H, m), 3.60 (2H, brs), 4.31 (2H, q, J=7.0 Hz), 7.13-7.33 (5H, m), 7.80 (2H, s), 8.51 (3H, d, J=4.9 Hz).

ESI-MS Found: m/z 437[M+H]⁺

### Example 3-11

### 5-[1-({6-[(E)-(3,4-Difluorophenyl)(ethoxyimino)methyl]pyridin-3-yl}methyl)piperidin-4-yl]-1-ethylpyridin-2(1H)-one dihydrochloride

¹ HNMR (400 MHz, DMSO-d₆, δ ppm): 1.17 (3H, t, J=7.1 Hz), 1.25 (3H, t, J=7.1 Hz), 1.86-1.88 (4H, m), 2.52-2.62 (1H, m), 2.93-3.03 (2H, m), 3.37-3.42 (2H, m), 3.86 (2H, q, J=7.1 Hz), 4.24 (2H, q, J=7.1 Hz), 4.35-4.39 (2H, m), 6.38 (1H, d, J=9.4 Hz), 7.17-7.20 (1H, m), 7.32 (1H, dd, J=9.4, 2.5 Hz), 7.41-7.55 (3H, m), 7.99 (1H, d, J=8.4 Hz), 8.19 (1H, dd, J=8.4, 2.2 Hz), 8.69 (1H, d, J=2.2 Hz).

ESI-MS Found: m/z 481 [M+H]⁺

### Example 3-12

### (E)-(3,4-Difluorophenyl)(5-{[4-(6-ethoxypyridin-3-yl)piperidin-1-yl)methyl]pyridin-2-yl)methanone O-ethyloxime

¹ HNMR (400 MHz, CDCl₃, δ ppm): 1.33 (3H, t, J=7.0 Hz), 1.38 (3H, t, J=7.1 Hz), 1.66-1.85 (4H, m), 2.06-2.19 (2H, m), 2.41-2.52 (1H, m), 2.93-3.03 (2H, m), 3.57 (2H, s), 4.28-4.34 (4H, m), 6.67 (1H, d, J=8.6 Hz), 7.13-7.34 (3H, m), 7.44 (1H, dd, J=8.6, 2.4 Hz), 7.71-7.83 (2H, m), 7.98 (1H, d, J=2.4 Hz), 8.51 (1H, s).

ESI-MS Found: m/z 481 [M+H]⁺

### Example 3-13

### 1-(Difluoromethyl)-5-[1-({6-[(E)-(3,4-difluorophenyl)(ethoxyimino)methyl]pyridin-3-yl}methyl)piperidin-4-yl]pyridin-2(1H)-one dihydrochloride

¹HNMR (400 MHz, DMSO-d₆, δ ppm): 1.25 (3H, t, J=7.1 Hz), 1.90-1.99 (4H, m), 2.64-2.74 (1H, m), 2.92-3.04 (2H, m), 3.34-3.42 (2H, m), 4.24 (3H, q, J=7.1 Hz), 4.38 (2H, d, J=4.4 Hz), 6.55 (1H, d, J=10.2 Hz), 7.16-7.21 (1H, m), 7.42-7.55 (4H, m), 7.83 (1H, t, J=60.0 Hz), 8.00 (1H, d, J=8.3 Hz), 8.17 (1H, dd, J=8.3, 2.4 Hz), 8.68 (1H, d, J=2.4 Hz).

ESI-MS Found: m/z 503[M+H]⁺

### Example 3-14

### E-3,4-Difluorophenyl){5-[4-pyridin-3-ylpiperidin-1-yl)methyl]pyridin-2-yl}methanone O-ethyloxime

¹HNMR (400 MHz, CDCl₃, δ ppm): 1.33 (3H, t, J=7.1 Hz), 1.80-1.88 (4H, m), 2.10-2.23 (2H, m), 2.50-2.61 (1H, m), 2.97-3.07 (2H, m), 3.60 (2H, brs), 4.31 (2H, q, J=7.1 Hz), 7.12-7.35 (4H, m), 7.52-7.55 (1H, m), 7.77-7.83 (2H, m), 8.45-8.52 (3H, m).

ESI-MS Found: m/z 437[M+H]⁺

### Example 3-15

### (E)-(3,4-Difluorophenyl){5-[(4-pyrimidin-5-ylpiperidin-1-yl)methyl]pyridin-2-yl}methanone O-ethyloxime

¹HNMR (400 MHz, CDCl₃, δ ppm): 1.33 (3H, t, J=7.1 Hz), 1.74-1.92 (4H, m), 2.09-2.22 (2H, m), 2.50-2.62 (1H, m), 2.96-3.08 (2H, m), 3.59 (2H, s), 4.31 (2H, q, J=7.1 Hz), 7.13-7.33 (4H, m), 7.70-7.84 (2H, m), 8.51 (1H, s), 8.62 (2H, s).

ESI-MS Found: m/z 438[M+H]⁺

### Example 3-16

### 5-[1-({6-[(E)-(3,4-Difluorophenyl)(ethoxyimino)methyl]pyridin-3-yl}methyl)piperidin-4-yl]nicotinonitrile

¹HNMR (400 MHz, CDCl₃, δ ppm): 1.33 (3H, t, J=7.1 Hz), 1.70-1.92 (4H, m), 2.07-2.24 (2H, m), 2.63 (1H, s), 3.01 (2H, s), 3.58 (2H, s), 4.31 (2H, q, J=7.1 Hz), 7.12-7.34 (3H, m), 7.69-7.85 (3H, m), 8.51 (1H, s), 8.69 (1H, d, J=2.4 Hz), 8.74 (1H, d, J=2.0 Hz).

ESI-MS Found: m/z 462[M+H]⁺

### Example 3-17

### (E)-(3,4-Difluorophenyl)(5-{[4-(1-methyl-1H-pyrazol-4-yl)piperidin-1-yl]methyl}pyridin-2-yl)methanone O-ethyloxime

¹HNMR (400 MHz, CDCl₃, δ ppm): 1.33 (3H, t, J=7.1 Hz), 1.56-1.70 (2H, m), 1.82-1.90 (2H, m), 2.05-2.15 (2H, m), 2.42-2.53 (1H, m), 2.87-2.94 (2H, m), 3.55 (2H, s), 3.85 (3H, s), 4.30 (2H, q, J=7.1 Hz), 7.11-7.24 (3H, m), 7.28-7.35 (2H, m), 7.72-7.80 (2H, m), 8.50 (1H, d, J=1.5 Hz).

ESI-MS Found: m/z 440[M+H]⁺

### Example 3-18

### (E)-(3,4-Difluorophenyl){5-[4-pyridazin-4-piperidin-1-yl)methyl]pyridin-2-yl}methanone O-ethyloxime

¹HNMR (400 MHz, CDCl₃, δ ppm): 1.33 (3H, t, J=7.1 Hz), 1.70-1.93 (4H, m), 2.09-2.24 (2H, m), 2.50-2.64 (1H, m), 2.95-3.09 (2H, m), 3.53-3.63 (2H, m), 4.31 (2H, q, J=7.1 Hz), 7.11-7.34 (4H, m), 7.65-7.87 (2H, m), 8.51 (1H, s), 9.07-9.12 (2H, m).

ESI-MS Found: m/z 438[M+H]⁺

### Example 3-19

### (E)-(3,4-Difluorophenyl)(5-{[4-(5-fluoropyridin-2-yl)piperidin-1-yl]methyl}pyridin-2-yl)methanone O-ethyloxime

¹ HNMR (400 MHz, CDCl₃, δ ppm): 1.33 (3H, t, J=7.1 Hz), 1.75-1.94 (4H, m), 2.10-2.19 (2H, m), 2.66-2.76 (1H, m), 2.95-3.02 (2H, m), 3.56 (2H, s), 4.30 (2H, q, J=7.1 Hz), 7.11-7.39 (5H, m), 7.73-7.80 (2H, m), 8.38 (1H, d, J=2.9 Hz), 8.51 (1H, s).

ESI-MS Found: m/z 455[M+H]⁺

### Example 3-20

### (E)-(3,4-Difluorophenyl){5-[(4-[1,2,4]triazolo[4,3-a]pyridin-6-ylpiperidin-1-yl)methyl]pyridin-2-yl)methanone O-ethyloxime

¹HNMR (400 MHz, CDCl₃, δ ppm): 1.33 (3H, t, J=7.1 Hz), 1.70-1.92 (4H, m), 2.11-2.20 (2H, m), 2.49-2.59 (1H, m), 2.99-3.05 (2H, m), 3.59 (2H, s), 4.31 (2H, q, J=7.0 Hz), 7.13-7.35 (4H, m), 7.70-7.82 (3H, m), 7.93 (1H, s), 8.52 (1H, d, J=1.5 Hz), 8.77 (1H, s).

ESI-MS Found: m/z 477[M+H]⁺

### Example 3-21

### (E)-(3,4-Difluorophenyl) {5-[(4-[1,2,4]triazolo[4,3-a]pyridin-7-ylpiperidin-1-yl)methyl]pyridin-2-yl)methanone O-ethyloxime

¹HNMR (400 MHz, CDCl₃, δ ppm): 1.33 (3H, t, J=7.1 Hz), 1.71-1.93 (4H, m), 2.12-2.20 (2H, m), 2.53-2.63 (1H, m), 2.99-3.05 (2H, m), 3.59 (2H, s), 4.31 (2H, q, J=7.2 Hz), 6.78 (1H, dd, J=7.1, 1.7 Hz), 7.13-7.34 (3H, m), 7.56 (1H, s), 7.74-7.82 (2H, m), 8.06-8.08 (1H, m), 8.52 (1H, d, J=1.5 Hz), 8.76 (1H, s).

ESI-MS Found: m/z 477[M+H]⁺

### Example 3-22

### 4-[1-({6-[(E)-(3,4-Difluorophenyl)(ethoxyimino)methyl]pyridin-3-yl)methyl)piperidin-4-yl]-1,3-dihydro-2H-indol-2-one

¹ HNMR (400 MHz, CDCl₃, δ ppm): 1.33 (3H, t, J=7.1 Hz), 1.68-1.83 (4H, m), 2.07-2.16 (2H, m), 2.41-2.50 (1H, m), 2.95-3.01 (2H, m), 3.51 (2H, s), 3.57 (2H, s), 4.30 (2H, q, J=7.2 Hz), 6.79 (1H, d, J=8.3 Hz), 7.03-7.24 (4H, m), 7.31 (1H, ddd, J=11.2, 7.8, 2.0 Hz), 7.73-7.81 (2H, m), 8.51 (1H, d, 1.5 Hz), 8.86 (1H, s).

ESI-MS Found: m/z 491[M+H]⁺

### Example 3-23

### 6-[1-({6-[(E)-(3,4-Difluorophenyl)(ethoxyimino)methyl]pyridin-3-yl)methyl)piperidin-4-yl]-1,3-dihydro-2H-indol-2-one

¹HNMR (400 MHz, CDCl₃, δ ppm): 1.33 (3H, t, J=7.1 Hz), 1.69-1.84 (4H, m), 2.07-2.16 (2H, m), 2.43-2.53 (1H, m), 2.95-3.01 (2H, m), 3.50 (2H, s), 3.58 (2H, s), 4.30 (2H, q, J=7.2 Hz), 6.76 (1H, s), 6.87 (1H, dd, J=7.8, 1.5 Hz), 7.12-7.24 (3H, m), 7.31 (1H, ddd, J=10.9, 7.9, 1.8 Hz), 7.74-7.81 (2H, m), 8.51 (1H, s), 8.85 (1H, s).

ESI-MS Found: m/z 491[M+H]⁺

### Example 3-24

### (E)-(3,4-Difluorophenyl){5-[(4-imidazo[1,2-a]pyridin-6-ylpiperidin-1-yl)methyl]pyridin-2-yl)methanone O-ethyloxime

¹ HNMR (400 MHz, CDCl₃, δ ppm): 1.33 (3H, t, J=7.1 Hz), 1.70-1.90 (4H, m), 2.10-2.18 (2H, m), 2.46-2.55 (1H, m), 2.97-3.03 (2H, m), 3.58 (2H, s), 4.31 (2H, q, J=7.0 Hz), 7.07-7.34 (4H, m), 7.52-7.59 (3H, m), 7.73-7.82 (2H, m), 7.93 (1H, s), 8.52 (1H, d, J=1.5 Hz).

ESI-MS Found: m/z 476[M+H]⁺

### Example 3-25

### (E)-(3,4-Difluorophenyl){5-[(4-[1,2,4]triazolo[1,5-a]pyridin-6-ylpiperidin-1-yl)methyl]pyridin-2-yl)methanone O-ethyloxime

¹HNMR (400 MHz, CDCl₃, δ ppm): 1.33 (3H, t, J=7.1 Hz), 1.73-1.95 (4H, m), 2.13-2.20 (2H, m), 2.57-2.67 (1H, m), 2.99-3.05 (2H, m), 3.59 (2H, s), 4.31 (2H, q, J=7.0 Hz), 7.14-7.34 (3H, m), 7.45 (1H, dd, j=9.0, 1.7 Hz), 7.69-7.83 (3H, m), 8.31 (1H, s), 8.43 (1H, s), 8.52 (1H, d, J=1.5 Hz).

ESI-MS Found: m/z 477[M+H]⁺

### Example 3-26

### (E)-(3,4-Difluorophenyl){5-[(4-imidazo[1,2-a]pyridin-7-ylpiperidin-1-yl)methyl]pyridin-2-yl)methanone O-ethyloxime

¹HNMR (400 MHz, CDCl₃, δ ppm): 1.33 (3H, t, J=7.1 Hz), 1.72-1.91 (4H, m), 2.10-2.18 (2H, m), 2.50-2.59 (1H, m), 2.97-3.03 (2H, m), 3.58 (2H, s), 4.31 (2H, q, J=7.0 Hz), 6.69 (1H, dd, J=7.1, 1.7 Hz), 7.13-7.35 (3H, m), 7.42 (1H, s), 7.51 (1H, s), 7.58 (1H, d, J=1.0 Hz), 7.74-7.82 (2H, m), 8.05 (1H, d, J=6.3 Hz), 8.52 (1H, d, 1.5 Hz).

ESI-MS Found: m/z 476[M+H]⁺

### Example 3-27

### (E)-(3,4-Difluorophenyl)(5-{[4-(2-methylimidazo[1,2-a]pyridin-6-yl)piperidin-1-yl]methyl}pyridin-2-yl)methanone O-ethyloxime

¹HNMR (400 MHz, CDCl₃, δ ppm): 1.33 (3H, t, J=7.1 Hz), 1.68-1.88 (4H, m), 2.08-2.17 (2H, m), 2.41-2.52 (4H, m), 2.96-3.02 (2H, m), 3.57 (2H, s), 4.31 (2H, q, J=7.2 Hz), 7.02 (1H, dd, J=9.3, 1.5 Hz), 7.13-7.35 (4H, m), 7.43 (1H, d, J=9.3 Hz), 7.73-7.83 (3H, m), 8.51 (1H, d, J=1.5 Hz).

ESI-MS Found: m/z 490[M+H]⁺

### Example 3-28

### (E)-(3,4-Difluorophenyl)(5-{[4-(2,3-dimethylimidazo[1,2-a]pyridin-6-yl)piperidin-1-yl]methyl}pyridin-2-yl)methanone O-ethyl oxime

¹HNMR (400 MHz, CDCl₃, δ ppm): 1.33 (3H, t, J=7.1 Hz), 1.73-1.90 (4H, m), 2.10-2.18 (2H, m), 2.37 (3H, s), 2.40 (3H, s), 2.48-2.58 (1H, m), 2.97-3.03 (2H, m), 3.58 (2H, s), 4.31 (2H, q, J=7.2 Hz), 7.02 (1H, dd, J=9.3, 2.0 Hz), 7.13-7.35 (3H, m), 7.44 (1H, d, J=9.3 Hz), 7.58 (1H, s), 7.82-7.74 (2H, m), 8.52 (1H, d, J=1.5 Hz).

ESI-MS Found: m/z 504[M+H]⁺

### Example 3-29

### (E)-(3,4-Difluorophenyl) {5-[(4-imidazo[1,2-a]pyrimidin-6-ylpiperidin-1-yl)methyl]pyridin-2-yl}methanone O-ethyloxime

¹HNMR (400 MHz, CDCl₃, δ ppm): 1.34 (3H, t, J=6.9 Hz), 1.73-1.97 (4H, m), 2.13-2.23 (2H, m), 2.57-2.68 (1H, m), 2.98-3.07 (2H, m), 3.60 (2H, s), 4.31 (2H, q, J=7.0 Hz), 7.13-7.35 (3H, m), 7.50 (1H, s), 7.73-7.83 (3H, m), 8.24 (1H, s), 8.54-8.46 (2H, m).

ESI-MS Found: m/z 477[M+H]⁺

### Example 3-30

### 6-[1-({6-[(E)-(3,4-Difluorophenyl)(ethoxyimino)methyl]pyridin-3-yl)methyl)piperidin-4-yl]-1-methyl-1,3-dihydro-2H-indol-2-one

¹HNMR (400 MHz, CDCl₃, δ ppm): 1.33 (3H, t, J=7.0 Hz), 1.74-1.87 (4H, m), 2.09-2.18 (2H, m), 2.49-2.58 (1H, m), 2.97-3.03 (2H, m), 3.20 (3H, s), 3.48 (2H, s), 3.58 (2H, s), 4.31 (2H, q, J=7.0 Hz), 6.70 (1H, s), 6.90 (1H, d, J=7.6 Hz), 7.13-7.35 (4H, m), 7.82-7.74 (2H, m), 8.53 (1H, s).

ESI-MS Found: m/z 505[M+H]⁺

### Example 3-31

### 5-[1-({6-[(E)-(3,4-Difluorophenyl)(ethoxyimino)methyl]pyridin-3-yl}methyl)piperidin-4-yl]-1,3-benzoxazol-2(3H)-one

¹HNMR (400 MHz, CDCl₃, δ ppm): 1.32 (3H, t, J=7.1 Hz), 1.67-1.86 (4H, m), 2.11-2.20 (2H, m), 2.48-2.58 (1H, m), 2.99-3.06 (2H, m), 3.64 (2H, s), 4.29 (2H, q, J=7.2 Hz), 6.95-6.90 (2H, m), 7.08-7.23 (3H, m), 7.31 (1H, ddd, J=11.2, 7.8, 2.0 Hz), 7.75-7.80 (2H, m), 8.53 (1H, s).

ESI-MS Found: m/z 493[M+H]⁺

### Example 3-32

### 5-[1-({6-[(E)-(3,4-Difluorophenyl)(ethoxylmino)methyl]pyridin-3-yl}methyl)piperidin-4-yl]-3-methyl-1,3-benzoxazol-2(3H)-one

¹HNMR (400 MHz, CDCl₃, δ ppm): 1.33 (3H, t, J=7.1 Hz), 1.72-1.87 (4H, m), 2.09-2.18 (2H, m), 2.51-2.60 (1H, m), 2.96-3.03 (2H, m), 3.39 (3H, s), 3.58 (2H, s), 4.31 (2H, q, J=7.2 Hz), 6.83 (1H, d, J=1.5 Hz), 6.96 (1H, dd, J=8.3, 2.0 Hz), 7.09-7.25 (3H, m), 7.31 (1H, ddd, J=11.1, 7.7, 1.8 Hz), 7.82-7.74 (2H, m), 8.53 (1H, d, J=1.0 Hz).

ESI-MS Found: m/z 507[M+14]⁺

### Example 3-33

### (E)-(3,4-Difluorophenyl)(5-{[4-(1-methyl-1H-pyrrolo[2,3-c]pyridin-3-yl)piperidin-1-yl]methyl}pyridin-2-yl)methanone O-ethyloxime

¹HNMR (400 MHz, CDCl₃, δ ppm): 1.33 (3H, t, J=7.0 Hz), 1.74-2.03 (4H, m), 2.15-2.23 (2H, m), 2.76-2.85 (1H, m), 2.95-3.02 (2H, m), 3.59 (2H, s), 3.85 (3H, s), 4.31 (2H, q, J=7.0 Hz), 6.96 (1H, s), 7.34-7.13 (3H, m), 7.51 (1H, d, J=5.5 Hz), 7.74-7.81 (2H, m), 8.22 (1H, d, J=5.5 Hz), 8.54 (1H, s), 8.72 (1H, s).

ESI-MS Found: m/z 490[M+H]⁺

### Example 3-34

### (E)-(3,4-Difluorophenyl){5-[(4-imidazo[1,2-a]pyrimidin-3-ylpiperidin-1-yl)methyl]pyridin-2-yl}methanone O-ethyloxime

¹HNMR (400 MHz, CDCl₃, δ ppm): 1.33 (3H, t, J=7.0 Hz), 1.79-2.28 (6H, m), 2.80-2.90 (1H, m), 2.97-3.05 (2H, m), 3.58-3.61 (2H, m), 4.30 (2H, q, J=7.0 Hz), 6.81 (1H, t, J=6.8 Hz), 7.10-7.42 (5H, m), 7.61 (1H, d, J=9.2 Hz), 7.74-7.83 (2H, m), 7.94 (1H, d, J=7.0 Hz), 8.52 (1H, s).

ESI-MS Found: m/z 476[M+H]⁺

### Example 3-35

### (E)-(3,4-Difluorophenyl){5-[(4-imidazo[1,2-a]pyrazin-3-ylpiperidin-1-yl)methyl]pyridin-2-yl}methanone O-ethyloxime

¹ HNMR (400 MHz, CDCl₃, δ ppm): 1.33 (3H, t, J=7.1 Hz), 1.82-2.28 (6H, m), 2.85-3.06 (3H, m), 3.60 (2H, s), 4.31 (2H, q, J=7.2 Hz), 7.13-7.35 (3H, m), 7.93-7.56 (5H, m), 8.52 (1H, d, J=2.0 Hz), 9.07 (1H, d, J=1.5 Hz).

ESI-MS Found: m/z 477[M+H]⁺

### Example 3-36

### (E)-(3,4-Difluorophenyl){5-[4-imidazo[1,2-b]pyridazin-3-ylpiperidin-1-yl)methyl]pyridin-2-yl}methanone O-ethyloxime

¹HNMR (400 MHz, CDCl₃, δ ppm): 1.33 (3H, t, J=7.1 Hz), 1.79-1.93 (2H, m), 2.11-2.29 (4H, m), 2.96-3.03 (2H, m), 3.14-3.24 (1H, m), 3.59 (2H, s), 4.30 (2H, q, J=7.2 Hz), 6.99 (1H, dd, J=8.8, 4.4 Hz), 7.13-7.35 (3H, m), 7.57 (1H, s), 7.75-7.82 (2H, m), 7.93 (1H, dd, J=9.0, 1.7 Hz), 8.31 (1H, dd, J=4.4, 2.0 Hz), 8.53 (1H, s).

ESI-MS Found: m/z 477[M+H]⁺

### Example 3-37

### (E)-(3,4-Difluorophenyl)(5-{[(3-endo)-3-[1,2,4]triazolo[4,3-a]pyridin-7-yl-8-azabicyclo[3.2.1]oct-8-yl]methyl}pyridin-2-yl}methanone O-ethyloxime

¹HNMR (400 MHz, CDCl₃, δ ppm): 1.34 (3H, t, J=7.1 Hz), 1.50-1.59 (4H, m), 2.06-2.12 (2H, m), 2.41-2.50 (2H, m), 3.12-3.22 (1H, m), 3.26-3.32 (2H, rya), 3.52 (2H, s), 4.31 (2H, q, J=7.1 Hz), 6.80-6.76 (1H, m), 7.14-7.35 (3H, m), 7.62 (1H, s), 7.80-7.81 (2H, m), 8.03 (1H, d, J=7.2 Hz), 8.61 (1H, s), 8.74 (1H, s).

ESI-MS Found: m/z 503[M+H]⁺

### Example 3-38

### (E)-(3,4-Difluorophenyl)(5-{[(3-exo)-3-[1,2,4]triazolo[4,3-a]pyridin-7-yl-8-azabicyclo[3.2.1]oct-8-yl]methyl}pyridin-2-yl}methanone O-ethyloxime

¹HNMR (400 MHz, CDCl₃, δ ppm): 1.33 (3H, t, J=7.0 Hz), 1.68-1.80 (4H, m), 1.85-1.94 (2H, m), 2.07-2.32 (4H, m), 2.90-3.00 (1H, m), 3.29-3.33 (2H, m), 3.61 (2H, s), 4.30 (2H, q, J=7.0 Hz), 6.80 (1H, d, J=7.0 Hz), 7.13-7.35 (3H, m), 7.56 (1H, s), 7.86-7.79 (2H, m), 8.07 (1H, d, J=7.2 Hz), 8.57 (1H, s), 8.76 (1H, s).

ESI-MS Found: m/z 503[M+H]⁺

### Example 3-39

### (E)-(3,4-Difluorophenyl)(5-[(2-methyl-4-[1,2,4]triazolo[4,3-a]pyridin-7-ylpiperidin-1-yl)methyl]pyridin-2-yl}methanone O-ethyloxime

¹HNMR (400 MHz, CDCl₃, δ ppm): 1.13-1.29 (3H, m), 1.31-1.36 (3H, m), 1.47-4.20 (1H, m), 4.27-4.34 (2H, m), 6.75-6.80 (1H, m), 7.13-7.25 (2H, m), 7.28-7.35 (1H, m), 7.52-7.59 (1H, m), 7.73-7.83 (2H, m), 8.05-8.10 (1H, m), 8.52-8.56 (1H, m), 8.75-8.78 (1H, m).

ESI-MS Found: m/z 491[M+H]⁺

### Example 3-40

### (Z)-(3,4-Difluorophenyl){4-[(4-pyrazin-2-ylpiperidin-1-yl)methyl]phenyl}methanone O-(2-fluoroethyl)oxime

¹HNMR (400 MHz, CDCl₃, δ ppm): 1.89-1.96 (4H, m), 2.10-2.20 (2H, m), 2.72-2.80 (1H, m), 3.00-3.06 (2H, m), 3.57 (2H, s), 4.41 (2H, dt, J=28.5, 4.3 Hz), 4.69 (2H, dt, J=47.5, 4.3 Hz), 7.10-7.15 (1H, m), 7.18-7.31 (2H, m), 7.33-7.42 (4H, m), 8.41 (1H, s), 8.50 (2H, s).

ESI-MS Found: m/z 455[M+H]⁺

### Example 3-41

### (Z)-(3,4-Difluorophenyl)(4-{[4-(2-methoxypyrimidin-5-yl)piperidin-1-yl]methyl}phenyl)methanone O-(2-fluoroethyl)oxime

¹HNMR (400 MHz, CDCl₃, δ ppm): 1.67-1.85 (4H, m), 2.06-2.17 (2H, m), 2.45-2.53 (1H, m), 2.98-3.07 (2H, m), 3.57 (2H, s), 3.99 (3H, s), 4.42 (2H, dt, J=28.5, 4.1 Hz), 4.69 (2H, dt, J=47.5, 4.1 Hz), 7.10-7.15 (1H, m), 7.18-7.30 (2H, m), 7.31-7.43 (4H, m), 8.38 (2H, s).

APCI-MS Found: m/z 485[M+H]⁺

### Example 3-42

### (Z)-(3,4-Difluorophenyl)(4-{[4-(2-fluoropyridin-5-yl)piperidin-1-yl]methyl}phenyl)methanone O-(2-fluoroethyl)oxime

¹ HNMR (400 MHz, CDCl₃, δ ppm): 1.74-1.89 (4H, m), 2.11-2.22 (2H, m), 2.76-2.86 (1H, m), 3.00-3.09 (2H, m), 3.60 (2H, s), 4.42 (2H, dt, J=28.5, 4.1 Hz), 4.69 (2H, dt, J=47.8, 4.1 Hz), 7.10-7.17 (2H, m), 7.18-7.31 (2H, m), 7.32-7.44 (4H, m), 7.62-7.69 (1H, m), 8.04-8.07 (1H, m).

ESI-MS Found: m/z 472[M+H]⁺

### Example 3-43

### (Z)-(3,4-Difluorophenyl)(4-{[4-(6-fluoropyridin-2-yl)piperidin-1-yl]methyl}phenyl)methanone O-(2-fluoroethyl)oxime

¹HNMR (400 MHz, CDCl₃, δ ppm): 1.79-1.97 (4H, m), 2.07-2.19 (2H, m), 2.61-2.71 (1H, m), 2.98-3.06 (2H, m), 3.57 (2H, s), 4.41 (2H, dt, J=28.6, 4.3 Hz), 4.69 (2H, dt, J=47.8, 4.3 Hz), 6.75 (1H, dd, J=8.0, 2.9 Hz), 7.05 (1H, dd, J=7.3, 2.4 Hz), 7.10-7.15 (1H, m), 7.17-7.45 (6H, m), 7.70 (1H, q, J=8.0 Hz).

ESI-MS Found: m/z 472[M+H]⁺

### Example 3-44

### 6-[1-(4-{(Z)-(3,4-Difluorophenyl)[(2-fluoroethxy)imino]methyl}benzyl)piperidin-4-yl]pyridin-2(1H)-one

¹ HNMR (400 MHz, CDCl₃, δ ppm): 1.71-1.84 (2H, m), 1.87-1.95 (2H, m), 2.10-2.21 (2H, m), 2.46-2.57 (1H, m), 2.97-3.04 (2H, m), 3.49 (1H, s), 3.57 (2H, s), 4.41 (2H, dt, J=28.5, 4.3 Hz), 4.69 (2H, dt, J=47.8, 4.3 Hz), 6.08 (1H, d, J=6.8 Hz), 6.42 (1H, d, J=9.3 Hz), 7.10-7.15 (1H, m), 7.19-7.42 (7H, m).

ESI-MS Found: m/z 470[M+H]⁺

### Example 3-45

### 5-[1-(4-{(Z)-(3,4-Difluorophenyl)[(2-fluoroethxy)imino]methyl}benzyl)piperidin-4-yl]pyrimidin-2(1H)-one

¹HNMR (400 MHz, CDCl₃, δ ppm): 1.65-1.86 (4H, m), 2.08-2.18 (2H, m), 2.36-2.46 (1H, m), 3.00-3.08 (2H, m), 3.59 (2H, s), 4.42 (2H, dt, J=28.5, 4.1 Hz), 4.69 (2H, dt, J=47.8, 4.1 Hz), 7.10-7.15 (1H, m), 7.18-7.45 (7H, m), 8.14 (1H, s).

ESI-MS Found: m/z 471[M+H]⁺

### Example 3-46

### 4-[1-(4-{(Z)-(3,4-Difluorophenyl)[(2-fluoroethxy)imino]methyl}benzyl)piperidin-4-yl]pyridin-2(1H)-one

¹HNMR (400 MHz, CDCl₃, δ ppm): 1.60-2.13 (4H, m), 2.30-2.41 (1H, m), 2.94-3.07 (2H, m), 3.57 (2H, s), 4.42 (2H, dt, J=28.3, 4.1 Hz), 4.69 (2H, dt, J=47.3, 4.1 Hz), 6.18 (1H, dd, J=6.8, 1.5 Hz), 6.40 (1H, s), 7.10-7.15 (1H, m), 7.18-7.44 (9H, m).

ESI-MS Found: m/z 470[M+H]⁺

### Example 3-47

### 6-[1-(4-{(Z)-(3,4-Difluorophenyl)[(2-fluoroethxy)imino]methyl}benzyl)piperidin-4-yl]-1-methylpyridin-2(1H)-one hydrochloride

¹ HNMR (400 MHz, DMSO-d₆, δ ppm): 1.31-1.37 (2H, m), 1.54-1.65 (2H, m), 1.87-2.06 (4H, m), 2.15-2.27 (3H, m), 3.65 (3H, s), 4.41 (3H, dt, J=28.8, 4.1 Hz), 4.65 (2H, dt, J=47.8, 4.1 Hz), 5.31-5.36 (1H, m), 6.29-6.56 (2H, m), 7.11-7.17 (1H, m), 7.27-7.43 (2H, m), 7.47-7.63 (4H, m).

ESI-MS Found: m/z 484[M+H]⁺

### Example 3-48

### (Z)-(3,4-Difluorophenyl)(4-{[4-(6-methoxypyridin-2-yl)piperidin-1-yl]methyl}phenyl)methanone O-(2-fluoroethyl)oxime

¹HNMR (400 MHz, CDCl₃, δ ppm): 1.25-1.37 (2H, m), 1.85-2.23 (4H, m), 2.52-2.66 (1H, m), 2.97-3.09 (2H, m), 160 (2H, s), 3.91 (3H, s), 4.41 (2H, dt, J=28.6, 4.1 Hz), 4.69 (2H, dt, J=47.6, 4.1 Hz), 6.54 (1H, d, J=8.3 Hz), 6.71 (1H, d, J=7.3 Hz), 7.10-7.15 (1H, m), 7.18-7.31 (2H, m), 7.33-7.52 (5H, m).

APCI-MS Found: m/z 484[M+H]⁺

### Example 3-49

### (E)-(3,4-Difluorophenyl)(5-{[4-(6-fluoropyridin-3-yl)piperidin-1-yl]methyl}pyridin-2-yl)methanone O-(2-hydroxy-2-methylpropyl)oxime

¹HNMR (400 MHz, CDCl₃, δ ppm): 1.24 (6H, s), 1.71-1.87 (4H, m), 2.08-2.21 (2H, m), 2.50-2.61 (2H, m), 2.94-3.05 (2H, m), 3.58 (2H, s), 4.14 (2H, s), 6.87 (1H, dd, J=8.2, 3.2 Hz), 7.14-7.34 (3H, m), 7.64 (1H, td, J=8.2, 2.6 Hz), 7.74-7.80 (2H, m), 8.06 (1H, d, J=2.6 Hz), 8.52 (1H, s).

ESI-MS Found: m/z 499[M+H]⁺

### Example 3-50

### N-(3-{1-[6-{(E)-(3,4-Difluorophenyl)[2-hydroxy-2-methylpropoxy)imino]methyl}piperidin-3-yl)methyl]piperidin-4-yl}-phenyl)-N-methylacetamide

¹HNMR (400 MHz, CDCl₃, δ ppm): 1.25 (6H, s), 1.70-1.90 (7H, m), 2.09-2.18 (2H, m), 2.48-2.58 (1H, m), 2.95-3.02 (2H, m), 3.25 (3H, s), 3.58 (2H, s), 4.15 (2H, s), 7.00-7.05 (2H, m), 7.15-7.36 (5H, m), 7.76-7.78 (2H, m), 8.53 (1H, s).

ESI-MS Found: m/z 551[M+H]⁺

### Example 3-51

### (E)-(3,4-Difluorophenyl){5-[(4-pyrazolo[1,5-b]pyridazin-3-ylpiperidin-1-yl)methyl]pyridin-2-yl}methanone O-(2-hydroxy-2-methylpropyl)oxime

¹HNMR (400 MHz, CDCl₃, δ ppm): 1.25 (6H, s), 1.81-1.97 (4H, m), 2.13-2.21 (2H, m), 2.58 (1H, s), 2.75-2.84 (1H, m), 2.95-3.01 (2H, m), 3.59 (2H, s), 4.15 (2H, s), 6.90 (1H, dd, J=8.8, 4.4 Hz), 7.15-7.35 (3H, m), 7.78-7.77 (2H, m), 7.89-7.94 (2H, m), 8.22 (1H, dd, J=4.4, 1.5 Hz), 8.53 (1H, s).

ESI-MS Found: m/z 521 [M+H]⁺

### Example 3-52

### (Z)-(3,4-Difluorophenyl)(5-{[4-(6-fluoropyridin-3-yl)piperidin-1-yl]methyl}pyridin-2-yl)methanone O-[(methylsulfonyl)methyl]oxime

¹HNMR (400 MHz, CDCl₃, δ ppm): 1.72-1.85 (4H, m), 2.13-2.19 (2H, m), 2.55-2.61 (1H, m), 2.95 (3H, s), 2.98-3.01 (2H, m), 3.60 (2H, s), 5.15 (2H, s), 6.89 (1H, dd, J=8.5, 2.8 Hz), 7.23-7.36 (3H, m), 7.64 (1H, td, J=8.1, 2.3 Hz), 7.80-7.80 (2H, m), 8.07-8.09 (1H, m), 8.57 (1H, s).

ESI-MS Found: m/z 519[M+H]⁺

### Example 3-53

### (E)-(3,4-Difluorophenyl)(5-{[4-(6-fluoropyridin-3-yl)piperidin-1-yl]methyl}pyridin-2-yl)methanone O-[(methylsulfonyl)methyl]oxime

¹HNMR (400 MHz, CDCl₃, δ ppm): 1.69-1.81 (4H, m), 2.08-2.14 (2H, m), 2.50-2.56 (1H, m), 2.90 (3H, s), 2.93-2.96 (2H, m), 3.55 (2H, s), 5.10 (2H, s), 6.84 (1H, dd, J=8.4, 2.9 Hz), 7.18-7.31 (3H, m), 7.60 (1H, td, J=8.1, 2.5 Hz), 7.74-7.76 (2H, m), 8.02-8.04 (1H, m), 8.52 (1H, s).

ESI-MS Found: m/z 519[M+H]⁺

### Example 3-54

### (E)-(3,4-Difluorophenyl)(5-{[4-(6-methylpyridin-3-yl)piperidin-1-yl]methyl}pyridin-2-yl)methanone O-[(methylsulfonyl)methyl]oxime

¹ HNMR (400 MHz, CDCl₃, δ ppm): 1.59-1.85 (4H, m), 2.10-2.19 (2H, m), 2.47-2.56 (4H, m), 2.93-3.01 (5H, m), 3.59 (2H, s), 5.14 (2H, s), 7.09 (1H, d, J=7.8 Hz), 7.20-7.35 (3H, m), 7.42 (1H, dd, J=7.8, 2.0 Hz), 7.75-7.82 (2H, m), 8.36 (1H, d, J=2.0 Hz), 8.56 (1H, s).

ESI-MS Found: m/z 515[M+H]⁺

### Example 3-55

### (Z)-(3,4-Difluorophenyl)(5-{[4-(6-methylpyridin-3-yl)piperidin-1-yl]methyl}pyridin-2-yl)methanone O-[(methylsulfonyl)methyl]oxime

¹HNMR (400 MHz, CDCl₃, δ ppm): 1.74-1.89 (4H, m), 2.15-2.24 (2H, m), 2.49-2.59 (4H, m), 2.98 (3H, s), 3.00-3.06 (2H, m), 3.63 (2H, s), 5.08 (2H, s), 7.08-7.22 (3H, m), 7.33-7.40 (1H, m), 7.44 (1H, dd, J=7.8, 2.4 Hz), 7.64 (1H, d, J=7.3 Hz), 7.89 (1H, dd, J=8.3, 2.0 Hz), 8.38 (1H, d, J=2.0 Hz), 8.68 (1H, s).

ESI-MS Found: m/z 515[M+H]⁺

### Example 3-56

### (E)-(3,4-Difluorophenyl){5-[(4-[1,2,4]triazolo[4,3-a]pyridin-7-ylpiperidin-1-yl)methyl]pyridin-2-yl}methanone O-cyclopropyloxime

¹HNMR (400 MHz, CDCl₃, δ ppm): 0.68-0.83 (4H, m), 1.71-1.93 (4H, m), 2.11-2.20 (2H, m), 2.53-2.63 (1H, m), 2.98-3.04 (2H, m), 3.59 (2H, s), 4.15-4.20 (1H, m), 6.78 (1H, dd, J=7.1, 1.7 Hz), 7.07-7.27 (3H, m), 7.56 (1H, s), 7.74-7.84 (2H, m), 8.06-8.09 (1H, m), 8.53 (1H, d, J=1.5 Hz), 8.77 (1H, s).

ESI-MS Found: m/z 489[M+H]⁺

### Example 3-57

### ({[(1E)-(3,4-Difluorophenyl)(5-{[4-(1-methyl-2-oxo-1,2-dihydropyridin-4-yl)piperidin-1-yl]methyl}pyridin-2-yl)methylene]amino}oxy)acetonitrile

¹HNMR (400 MHz, CDCl₃, δ ppm): 1.67-1.83 (4H, m), 2.06-2.18 (2H, m), 2.28-2.38 (1H, m), 2.91-3.01 (2H, m), 3.51 (3H, s), 3.58 (2H, s), 4.86 (2H, s), 6.07 (1H, d, J=5.7 Hz), 6.41 (1H, s), 7.10-7.16 (1H, m), 7.19-7.28 (3H, m), 7.84 (2H, t, J=18.0 Hz), 8.53 (1H, s).

ESI-MS Found: m/z 478[M+H]⁺

### Example 3-58

### ({[(1E)-(3,4-Difluorophenyl)(5-{[4-(6-fluoropyridin-3-yl)piperidin-1-yl]methyl}pyridin-2-yl)methylene]amino}oxy)acetonitrile

¹HNMR (400 MHz, CDCl₃, δ ppm): 1.69-1.87 (4H, m), 2.09-2.21 (2H, m), 2.51-2.61 (1H, m), 2.95-3.04 (2H, m), 3.60 (2H, s), 4.86 (2H, s), 6.88 (1H, dd, J=8.4, 2.9 Hz), 7.11-7.16 (1H, m), 7.21-7.30 (2H, m), 7.61-7.67 (1H, m), 7.76-7.83 (1H, m), 7.86-7.91 (1H, m), 8.07 (1H, s), 8.54 (1H, s).

ESI-MS Found: m/z 466[M+H]⁺

### Example 3-59

### ({[(1E)-(3,4-Difluorophenyl)(5-{[4-(2-fluoropyridin-4-yl)piperidin-1-yl]methyl}pyridin-2-yl)methylene]amino}oxy)acetonitrile

¹ HNMR (400 MHz, CDCl₃, δ ppm): 1.67-1.93 (4H, m), 2.08-2.21 (2H, m), 2.50-2.66 (1H, m), 2.93-3.06 (2H, m), 3.59 (2H, brs), 4.86 (2H, s), 6.78 (1H, s), 7.02-7.06 (1H, m), 7.10-7.16 (1H, m), 7.21-7.30 (2H, m), 7.72-7.95 (2H, m), 8.13 (1H, d, J=4.9 Hz), 8.54 (1H, s).

ESI-MS Found: m/z 466[M+H]⁺

### Example 3-60

### ({[(1Z)-(3,4-Difluorophenyl)(5-{[4-(6-fluoropyridin-3-yl)piperidin-1-yl]methyl}pyridin-2-yl)methylene]amino}oxy)acetonitrile

¹ HNMR (400 MHz, CDCl₃, δ ppm): 1.74-1.91 (4H, m), 2.14-2.25 (2H, m), 2.55-2.66 (1H, m), 3.00-3.09 (2H, m), 3.63 (2H, s), 4.81 (2H, s), 6.89 (1H, dd, J=8.0, 2.9 Hz), 7.11-7.18 (1H, m), 7.21-7.25 (1H, m), 7.36-7.42 (1H, m), 7.49 (1H, d, J=7.8 Hz), 7.66 (1H, td, J=8.0, 2.4 Hz), 7.83-7.90 (1H, m), 8.09 (1H, d, J=2.4 Hz), 8.68 (1H, d, J=1.5 Hz).

ESI-MS Found: m/z 466[M+H]⁺

### Example 3-61

### ({[(1E)-(3,4-Difluorophenyl)(5-{[4-5-fluoropyridin-3-yl)piperidin-1-yl]methyl}pyridin-2-yl)methylene]amino}oxy)acetonitrile

¹HNMR (400 MHz, CDCl₃, δ ppm): 1.71-1.90 (4H, m), 2.10-2.24 (2H, m), 2.54-2.67 (1H, m), 2.96-3.07 (2H, m), 3.60 (2H, s), 4.86 (2H, s), 7.10-7.17 (1H, m), 7.20-7.31 (3H, m), 7.76-7.93 (2H, m), 8.32 (2H, s), 8.54 (1H, s).

ESI-MS Found: m/z 466[M+H]⁺

### Example 3-62

### {[((1E)-(3,4-Difluorophenyl){5-[4-[1,2,4]triazolo[1,5-a]pyridin-7-ylpiperidin-1-yl)methyl]pyridin-2-yl}methylene)amino]oxy}acetonitrile

¹HNMR (400 MHz, CDCl₃, δ ppm): 1.66-1.95 (4H, m), 2.14-2.23 (2H, m), 2.61-2.70 (1H, m), 2.99-3.05 (2H, m), 3.61 (2H, s), 4.86 (2H, s), 6.93 (1H, dd, J=7.1, 1.7 Hz), 7.11-7.16 (1H, m), 7.20-7.30 (2H, m), 7.57 (1H, s), 7.78-7.91 (2H, m), 8.30 (1H, s), 8.56-8.50 (2H, m).

ESI-MS Found: m/z 488[M+H]⁺

### Example 3-63

### (E)-(3,4-Difluorophenyl)(5-{[4-(6-fluoropyridin-3-yl)piperidin-1-yl]methyl}pyridin-2-yl)methanone O-[(2S)-2-hydroxypropyl]oxime

¹HNMR (400 MHz, CDCl₃, δ ppm): 1.21 (3H, d, J=6.3 Hz), 1.68-1.86 (4H, m), 2.08-2.18 (2H, m), 2.45 (1H, brs), 2.50-2.61 (1H, m), 2.94-3.03 (2H, m), 3.57 (2H, s), 4.06-4.26 (3H, m), 6.87 (1H, dd, J=8.8, 2.9 Hz), 7.12-7.34 (3H, m), 7.63 (1H, td, J=8.0, 2.4 Hz), 7.75 (2H, s), 8.06 (1H, d, J=2.4 Hz), 8.52 (1H, s).

ESI-MS Found: m/z 485[M+H]⁺

### Example 3-64

### (Z)-(3,4-Difluorophenyl)(5-{[4-(6-fluoropyridin-3-yl)piperidin-1-yl]methyl}pyridin-2-yl)methanone O-[(2S)-2-hydroxypropyl]oxime

¹HNMR (400 MHz, CDCl₃, δ ppm): 1.17 (3H, d, J=6.3 Hz), 1.72-1.89 (4H, m), 2.13-2.25 (2H, m), 2.54-2.65 (1H, m), 2.98-3.08 (2H, m), 3.62 (2H, s), 3.91 (1H, dd, J=11.7, 8.8 Hz), 4.08-4.20 (1H, m), 4.25 (1H, dd, J=11.7, 2.9 Hz), 6.88 (1H, dd, J=8.2, 2.9 Hz), 7.09-7.20 (2H, m), 7.32-7.40 (2H, m), 7.65 (1H, td, J=8.2, 2.4 Hz), 7.85 (1H, d, J=6.8 Hz), 8.08 (1H, d, J=2.4 Hz), 8.67 (1H, d, J=1.5 Hz).

ESI-MS Found: m/z 485[M+H]⁺

### Example 3-65

### (E)-(3,4-Difluorophenyl)(5-{[4-6-fluoropyridin-3-yl)piperidin-1-yl]methyl}pyridin-2-yl)methanone O-[(2R)-2-hydroxypropyl]oxime

¹HNMR (400 MHz, CDCl₃, δ ppm): 1.21 (3H, d, J=6.3 Hz), 1.68-1.86 (4H, m), 2.07-2.18 (2H, m), 2.46 (1H, brs), 2.50-2.61 (1H, m), 2.94-3.03 (2H, m), 3.57 (2H, s), 4.06-4.26 (3H, m), 6.87 (1H, dd, J=8.0, 2.9 Hz), 7.12-7.34 (3H, m), 7.63 (1H, td, J=8.0, 2.4 Hz), 7.75 (2H, s), 8.06 (1H, d, J=2.4 Hz), 8.52 (1H, s).

ESI-MS Found: m/z 485[M+H]⁺

### Example 3-66

### (Z)-(3,4-Difluorophenyl)(5-{[4-(6-fluoropyridin-3-yl)piperidin-1-yl]methyl}pyridin-2-yl)methanone O-[(2R)-2-hydroxypropyl]oxime

¹HNMR (400 MHz, CDCl₃, δ ppm): 1.17 (3H, d, J=6.3 Hz), 1.72-1.90 (4H, m), 2.13-2.24 (2H, m), 2.53-2.65 (1H, m), 2.99-3.07 (2H, m), 3.62 (2H, s), 3.91 (1H, dd, J=11.5, 8.8 Hz), 4.11-4.20 (1H, m), 4.25 (1H, dd, J=11.5, 2.7 Hz), 6.88 (1H, dd, J=8.0, 2.9 Hz), 7.08-7.20 (2H, m), 7.32-7.40 (2H, m), 7.65 (1H, td, J=8.0, 2.4 Hz), 7.82-7.87 (1H, m), 8.08 (1H, d, J=2.4 Hz), 8.67 (1H, d, J=1.5 Hz).

ESI-MS Found: m/z 485 [M+H]⁺

### Example 3-67

### 5-[1-(1-{6-[(E)-(3,4-Difluorophenyl)(ethoxyimino)methyl]pyridin-3-yl}ethyl)piperidin-4-yl]-1-methylpyridin-2(1H)-one

Title compound AD: eluted earlier

¹HNMR (400 MHz, CDCl₃, δ ppm): 1.33 (3H, t, J=7.1 Hz), 1.40 (3H, d, J=6.8 Hz), 1.48-1.82 (4H, m), 1.94-2.12 (2H, m), 2.15-2.25 (1H, m), 2.84-2.92 (1H, m), 3.07-3.15 (1H, m), 3.51-3.59 (4H, m), 4.30 (2H, q, J=7.2 Hz), 6.54 (1H, d, J=9.3 Hz), 7.05 (1H, d, J=2.4 Hz), 7.14-7.35 (4H, m), 7.82-7.70 (2H, m), 8.52 (1H, d, J=2.0 Hz).

ESI-MS Found: m/z 481[M+H]⁺

Title compound AD: eluted later

¹HNMR (400 MHz, CDCl₃, δ ppm): 1.33 (3H, t, J=7.1 Hz), 1.40 (3H, d, J=6.8 Hz), 1.48-1.82 (4H, m), 1.94-2.11 (2H, m), 2.15-2.25 (1H, m), 2.84-2.91 (1H, m), 3.07-3.15 (1H, m), 3.52-3.59 (4H, m), 4.31 (2H, q, J=7.0 Hz), 6.54 (1H, d, J=, 9.3 Hz), 7.05 (1H, d, J=2.4 Hz), 7.14-7.34 (4H, m), 7.82-7.70 (2H, m), 8.52 (1H, d, J=2.0 Hz).

ESI-MS Found: m/z 481[M+H]⁺

### Example 3-68

### 4-[1-({5-[(E)-(3,4-Difluorophenyl)(ethoxyimino)methyl]pyridin-2-yl}methyl)piperidin-4-yl]-1-methylpyridin-2(1H)-one

¹ HNMR (400 MHz, CDCl₃, δ ppm): 1.32 (3H, t, J=7.1 Hz), 1.67-1.82 (4H, m), 2.13-2.21 (2H, m), 2.30-2.40 (1H, m), 2.98-3.04 (2H, m), 3.51 (3H, s), 3.70 (2H, s), 4.27 (2H, q, J=7.2 Hz), 6.08 (1H, dd, J=7.3, 2.0 Hz), 6.42 (1H, d, J=1.5 Hz), 7.08-7.13 (1H, m), 7.20-7.33 (3H, m), 7.43 (1H, d, J=8.3 Hz), 7.76 (1H, dd, J=8.0, 2.2 Hz), 8.60 (1H, d, J=2.0 Hz).

ESI-MS Found: m/z 467[M+H]⁺

### Example 3-69

### (E)-(3,4-Difluorophenyl)(6-{[4-(1-oxydopyridin-3-yl)piperidin-1-yl}methyl]pyridin-3-yl)methanone O-ethyloxime

¹ HNMR (400 MHz, CDCl₃, δ ppm): 1.32 (3H, t, J=7.1 Hz), 1.80-1.88 (4H, m), 2.16-2.28 (2H, m), 2.47-2.57 (1H, m), 3.00-3.10 (2H, m), 3.73 (2H, s), 4.27 (2H, q, J=7.1 Hz), 7.08-7.12 (1H, m), 7.16-7.32 (4H, m), 7.40-7.47 (1H, m), 7.77 (1H, dd, J=8.0, 2.2 Hz), 8.07-8.10 (1H, m), 8.13 (1H, s), 8.61 (1H, d, J=2.0 Hz).

ESI-MS Found: m/z 453[M+H]⁺

### Example 3-70

### (E)-(3,4-Difluorophenyl)(6-{[4-(1-oxydopyridin-4-yl)piperidin-1-yl]methyl}pyridin-3-yl)methanone O-ethyloxime

¹HNMR (400 MHz, CDCl₃, δ ppm): 1.32 (3H, t, J=7.1 Hz), 1.73-1.91 (4H, m), 2.15-2.28 (2H, m), 2.49-2.62 (1H, m), 2.98-3.10 (2H, m), 3.72 (2H, brs), 4.28 (2H, q, J=7.1 Hz), 7.07-7.17 (3H, m), 7.20-7.32 (2H, m), 7.37-7.47 (1H, m), 7.73-7.81 (1H, m), 8.12-8.16 (2H, m), 8.62 (1H, s).

ESI-MS Found: m/z 453[M+H]⁺

### Example 3-71

### 5-{1-[(5-{(E)-(3,4-Difluorophenyl)[(2,2,2-trifluoroethoxy)imino]methyl}pyridin-2-yl)methyl]piperidin-4-yl}-1-methylpyridin-2(1H)-one

¹HNMR (400 MHz, CDCl₃, δ ppm): 1.72-1.79 (4H, m), 2.12-2.35 (3H, m), 2.95-3.06 (2H, m), 3.52 (3H, s), 3.69-3.76 (2H, m), 4.56 (2H, q, J=8.5 Hz), 6.56 (1H, d, J=9.3 Hz), 7.04-7.11 (2H, m), 7.20-7.31 (3H, m), 7.43-7.50 (1H, m), 7.76 (1H, dd, J=7.8, 2.0 Hz), 8.60 (1H, d, J=2.0 Hz).

ESI-MS Found: m/z 521[M+H]⁺

### Example 3-72

### (Z)-(3,4-Difluorophenyl){4-[(4-imidazo[1,2-a]pyrimidin-3-ylpiperidin-1-yl)methyl]phenyl}methanone O-(2-hydroxy-2-methylpropyl)oxime

¹HNMR (400 MHz, CDCl₃, δ ppm): 1.23 (6H, s), 1.82-2.23 (6H, m), 2.79-3.06 (4H, m), 3.58 (2H, s), 4.08 (2H, s), 6.88 (1H, dd, J=6.8, 3.9 Hz), 7.10-7.15 (1H, m), 7.21-7.41 (6H, m), 7.60 (1H, s), 8.29 (1H, dd, J=7.1, 2.2 Hz), 8.52 (1H, dd, J=4.1, 2.2 Hz).

ESI-MS Found: m/z 520[M+H]⁺

### Example 3-73

### ({[(1E)-(3,4-Difluorophenyl)(4-{[4-(1-oxidopyridin-4-yl)piperidin-1-yl]methyl}phenyl)methylene]amino}oxy)acetonitrile

¹HNMR (400 MHz, CDCl₃, δ ppm): 1.71-1.91 (4H, m), 2.09-2.20 (2H, m), 2.50-2.63 (1H, m), 3.01-3.13 (2H, m), 3.55-3.65 (2H, m), 4.80 (2H, s), 7.13-7.18 (3H, m), 7.21-7.31 (3H, m), 7.35-7.51 (3H, m), 8.15 (2H, d, J=7.3 Hz).

ESI-MS Found: m/z 463[M+H]⁺

### Example 3-74

### ({[(1Z)-(3,4-Difluorophenyl)(4-{[4-(1-oxidopyridin-4-yl)piperidin-1-yl]methyl}phenyl)methylene]amino}oxy)acetonitrile

¹HNMR (400 MHz, CDCl₃, δ ppm): 1.68-1.89 (2H, m), 2.04-2.19 (2H, m), 2.47-2.60 (1H, m), 2.95-3.10 (2H, m), 3.53-3.65 (2H, m), 4.81 (2H, s), 7.07-7.10 (1H, m), 7.14 (2H, d, J=6.8 Hz), 7.18-7.29 (5H, m), 7.32-7.49 (3H, m), 8.14 (2H, d, J=7.3 Hz).

ESI-MS Found: m/z 463[M+H]⁺

### Example 3-75

### 5-(1-{4-[(E)-(5-Chloropyridin-2-yl)(ethoxyimino)methyl]benzyl}piperidin-4-yl}-1-methylpyridin-2(1H)-one

¹HNMR (400 MHz, CDCl₃, δ ppm): 1.34 (31-1, t, J=7.1 Hz), 1.61-1.81 (4H, m), 2.03-2.15 (2H, m), 2.23-2.33 (1H, m), 3.04-3.11 (2H, m), 3.53 (3H, s), 3.59 (2H, s), 4.30 (2H, q, J=7.1 Hz), 6.56 (1H, d, J=9.3 Hz), 7.07 (1H, d, J=2.4 Hz), 7.29 (1H, dd, J=9.3, 2.4 Hz), 7.35-7.42 (4H, m), 7.66-7.77 (2H, m), 8.53 (1H, dd, J=2.4, 1.0 Hz).

ESI-MS Found: m/z 465[M+H]⁺

### Example 3-76

### 5-(1-{4-[(Z)-(5-Chloropyridin-2-yl)(ethoxyimino)methyl]benzyl}piperidin-4-yl]-1-methylpyridin-2(1H)-one

¹ HNMR (400 MHz, CDCl₃, δ ppm): 1.29 (3H, t, J=7.0 Hz), 1.57-1.77 (4H, m), 2.00-2.09 (2H, m), 2.19-2.30 (1H, m), 2.96-3.03 (2H, m), 3.52 (3H, s), 3.55 (2H, s), 4.25 (2H, q, J=7.0 Hz), 6.56 (1H, d, J=9.3 Hz), 7.05 (1H, d, J=2.4 Hz), 7.25-7.32 (3H, m), 7.41 (2H, d, J=7.8 Hz), 7.55 (1H, d, J=8.3 Hz), 7.78 (1H, dd, J=8.3, 2.4 Hz), 8.67 (1H, d, J=2.4 Hz).

ESI-MS Found: m/z 465[M+H]⁺

### Example 3-77

### (E)-(5-Chloropyridin-2-yl){4-[4-[1,2,4]triazolo[4,3-a]pyridin-7-ylpiperidin-1-yl)methyl]phenyl}methanone O-ethyloxime

¹ HNMR (400 MHz, CDCl₃, δ ppm): 1.34 (3H, t, J=7.1 Hz), 1.75-1.93 (4H, m), 2.11-2.19 (2H, m), 2.54-2.64 (1H, m), 3.06-3.13 (2H, m), 3.59 (2H, s), 4.31 (2H, q, J=7.0 Hz), 6.79 (1H, dd, J=7.3, 1.5 Hz), 7.44-7.37 (4H, m), 7.57 (1H, s), 7.68 (1H, dd, J=8.8, 2.4 Hz), 7.75 (1H, d, J=7.8 Hz), 8.06 (1H, d, J=7.8 Hz), 8.53 (1H, d, J=2.0 Hz), 8.75 (1H, s).

ESI-MS Found: m/z 475[M+H]⁺

### Example 3-78

### (Z)-(5-Chloropyridin-2-yl){4-[4-[1,2,4]triazolo[4,3-a]pyridin-7-ylpiperidin-1-yl)methyl]phenyl}methanone O-ethyloxime

¹HNMR (400 MHz, CDCl₃, δ ppm): 1.29 (3H, t, J=6.8 Hz), 1.71-1.90 (4H, m), 2.06-2.15 (2H, m), 2.51-2.60 (1H, m), 2.99-3.06 (2H, m), 3.56 (2H, s), 4.25 (2H, q, J=7.2 Hz), 6.78 (1H, dd, J=7.3, 1.5 Hz), 7.31-7.33 (2H, m), 7.41-7.43 (2H, m), 7.54-7.55 (2H, m), 7.78 (1H, dd, 8.3, 2.4 Hz), 8.07-8.04 (1H, m), 8.67 (1H, d, J=2.4 Hz), 8.75 (1H, s).

ESI-MS Found: m/z 475[M+H]⁺

### Example 3-79

### (E)-(6-Chloropyridin-3-yl)(4-{[4-(6-fluoropyridin-3-yl)piperidin-1-yl]methyl}phenyl)methanone O-(2-hydroxy-2-methylpropyl)oxime

¹ HNMR (400 MHz, CDCl₃, δ ppm): 1.23 (6H, s), 1.71-1.87 (4H, m), 2.09-2.18 (2H, m), 2.52-2.62 (1H, m), 3.01-3.07 (2H, m), 3.60 (2H, s), 4.10 (2H, s), 6.87 (1H, dd, J=8.8, 2.9 Hz), 7.30-7.35 (4H, m), 7.43-7.47 (2H, m), 7.62-7.68 (1H, m), 7.81 (1H, dd, J=8.3, 2.4 Hz), 8.08 (1H, d, J=2.4 Hz), 8.39 (1H, d, J=2.4 Hz).

ESI-MS Found: m/z 497[M+H]⁺

### Example 3-80

### (Z)-(6-Chloropyridin-3-yl)(4-{[4-(6-fluoropyridin-3-yl)piperidin-1-yl]methyl}phenyl)methanone O-(2-hydroxy-2-methylpropyl)oxime

¹ HNMR (400 MHz, CDCl₃, δ ppm): 1.23 (6H, s), 1.69-1.85 (4H, m), 2.06-2.14 (2H, m), 2.50-2.60 (1H, m), 2.96-3.03 (2H, m), 3.56 (2H, s), 4.10 (2H, s), 6.87 (1H, dd, J=8.8, 2.9 Hz), 7.32-7.45 (6H, m), 7.64 (1H, td, J=8.3, 2.9 Hz), 7.71 (1H, dd, J=8.3, 2.4 Hz), 8.06 (1H, d, J=2.4 Hz), 8.44 (1H, d, J=2.4 Hz).

ESI-MS Found: m/z 497[M+H]⁺

### Example 4-1

### Synthesis of (E)-(3,4-difluorophenyl)(5-{[4-(6-fluoropyridin-3-yl)piperidin-1-yl]methyl}pyrimidin-2-yl)methanone-O-cyclopropyloxime

To an ethyl acetate solution (2.00 ml) of 2-fluoro-5-piperidin-4-ylpyridine (126 mg) obtained in Referential Example 3, potassium carbonate (1000 mg) was added and the resultant mixture was stirred at room temperature and then dried. Next, the solvent was distilled off under reduced pressure. To a methanol solution (2.00 ml) of the obtained residue and the compound (106 mg) obtained in Referential Example 7-6, cyanohydroborate and zinc chloride (II) were added at 0° C. and the resultant mixture was stirred at room temperature overnight. After cooling to 0° C., a 2N aqueous sodium hydroxide solution was added to the liquid reaction mixture and then the organic layer was extracted with ethyl acetate. The organic layer was dried over anhydrous magnesium sulfate and then concentrated under reduced pressure. The residue was purified by silica gel column chromatography (NII silica gel, n-hexane:ethyl acetate=100:0 to 50:50). Thus, the title compound (62.6 mg) was obtained as a white amorphous substance.

¹ HNMR (400 MHz, CDCl₃, δ ppm): 0.70-0.84 (4H, m), 1.67-1.89 (4H, m), 2.12-2.21 (2H, m), 2.49-2.62 (1H, m), 2.93-3.00 (2H, m), 3.58 (2H, s), 4.27-4.33 (1H, m), 6.88 (1H, dd, J=8.3, 2.9 Hz), 7.08-7.13 (1H, m), 7.17-7.28 (2H, m), 7.63 (1H, td, J=8.3, 2.6 Hz), 8.07 (1H, s), 8.77 (2H, s).

ESI-MS Found: m/z 468[MA-1]⁺

### INDUSTRIAL APPLICABILITY

Because of having MCH-1R antagonism, the compounds of the invention are useful as preventives or remedies for, e.g., metabolic disorders such as obesity, diabetes, hormone disorder, hyperlipidemia, gout, fatty liver, hepatitis, cirrhosis and so on; circulatory diseases such as stenocardia, acute or congestive heart failure, myocardial infarction, coronary atherosclerosis, hypertension, renal diseases, electrolyte abnormality and so on; central/peripheral nervous system disorders such as bulimia, emotional disturbance, depression, anxiety, epilepsy, delirium, dementia, schizophrenia, attention-deficit hyperactivity disorder, memory impairment, sleep disorders, cognitive failure, dyskinesia, paresthesias, smell disorders, morphine tolerance, drug dependence, alcoholism and so on; reproductive disorders such as infertility, preterm labor, sexual dysfunction and so on; digestive disorders; respiratory disorders; cancer or pigmentation and so on and, in particular, as preventives or remedies for obesity, diabetes, fatty liver, bulimia, depression and anxiety.

1-19. (canceled)

20. A compound represented by the formula (I) or a pharmaceutically acceptable salt thereof: 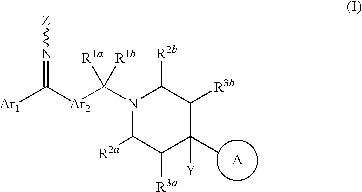
wherein,
R^{1a} and R^{1b} each independently represent a hydrogen atom or a C₁₋₆ alkyl group optionally having a substituent;
R^{2a} and R^{2b} each independently represents a hydrogen atom or a C₁₋₆ alkyl group optionally having a substituent, or R^{2a} and R^{2b} form together —C(R⁴)₂—C(R⁵)₂—;
R^{3a} and R^{3b} each independently represents a hydrogen atom or a C₁₋₆ alkyl group optionally having a substituent, or R^{3a} and R^{3b} form together —C(R⁶)₂—C(R⁷)₂—;
R⁴, R⁵, R⁶ and R⁷ each independently represent a hydrogen atom or a C₁₋₆ alkyl group optionally having a substituent;
Y represents a hydrogen atom or a hydroxy group;
Z represents —OR⁸, —N(R^{9a})(R^{9b}), —N(R¹⁰)—COO(R¹¹), —N(R¹²)—CO(R¹³), —C(R^{14a})(R^{14b})(R^{14c}), —O—SO₂R¹⁵ or —SO₂R¹⁶;
R⁸ represents a hydrogen atom, a C₁₋₆ alkyl group optionally having a substituent or a C₃₋₈ cycloalkyl group optionally having a substituent, wherein the C₁₋₆ alkyl group or C₃₋₈ cycloalkyl group is optionally substituted by a substituent selected from the group consisting of halogen, hydroxy, C₁₋₆ alkoxy, C₁₋₆ alkoxycarbonyl, C₁₋₆ alkylsulfonyl, (C₁₋₆ alkyl)amino, di(C₁₋₆ alkyl)amino, carbamoyl, (C₁₋₆ alkyl)carbamoyl, di(C₁₋₆ alkyl)carbamoyl and cyano groups;
R^{9a}, R^{9b}, R¹⁰, R¹¹, R¹², R¹³, R^{14a}, R^{14b} and R^{14c} each independently represent a hydrogen atom or a C₁₋₆ alkyl group optionally having a substituent;
R¹⁵ and R¹⁶ each independently represent a C₁₋₆ alkyl group or a phenyl group optionally substituted by a C₁₋₆ alkyl group;
Ar₁ represents an aromatic carbon ring group optionally substituted by a substituent selected from the group α, or an nitrogen-containing aromatic heterocyclic group optionally substituted by a substituent selected from the group α;
Ar₂ represents a group formed by removing two hydrogen atoms from an aromatic carbon ring group or an aromatic heterocyclic group wherein the aromatic carbon ring group or aromatic heterocyclic group is optionally substituted by a substituent selected from the group α; and 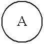
in the formula (I) (hereinafter referred to the ring group A) represents
1) an aromatic carbon ring group or a partially unsaturated carbon ring group, or
2) an aromatic heterocyclic group or a partially unsaturated heterocyclic group, wherein the group is optionally substituted by a substituent selected from the group consisting of halogen, C₁₋₆ alkyl, C₁₋₆ haloalkyl, C₁₋₆ alkyloxy, C₁₋₆ alkylcarbonylamino, C₁₋₆ alkylcarbonyl-C₁₋₆ alkylamino, cyano and oxo groups;
substituents of the group α:
halogen, cyano, hydroxy, amino, mono(C₁₋₆ alkyl)amino, alkyl)amino, C₁₋₆ alkyl, C₁₋₆ haloalkyl, C₁₋₆ alkyloxy, C₁₋₆haloalkyloxy, C₁₋₆ alkyloxy-C₁₋₆ alkyl, C₁₋₆ alkyloxycarbonyl, C₁₋₆ alkyloxycarbonylamino, C₁₋₆ alkyloxycarbonyl(C₁₋₆ alkyl)amino, C₁₋₆ alkylcarbonyl, C₁₋₆ alkylcarbonyloxy, C₁₋₆ alkylcarbonylamino, C₁₋₆ alkylcarbonyl(C₁₋₆ alkyl)amino, carbamoyl, mono(C₁₋₆ alkyl)carbamoyl, alkyl)carbamoyl, carbamoylamino, mono(C₁₋₆ alkyl)carbamoylamino, alkyl)carbamoylamino, mono(C₁₋₆ alkyl)carbamoyl(C₁₋₆ alkyl)amino, alkyl)carbamoyl(C₁₋₆ alkyl)amino, carbamoyloxy, mono(C₁₋₆ alkyl)carbamoyloxy, alkyl)carbamoyloxy, C₁₋₆ alkylsulfonyl, C₁₋₆ alkylsulfonylamino, C₁₋₆ alkylsulfonyl(C₁₋₆ alkyl)amino, sulfamoyl, mono(C₁₋₆ alkyl)sulfamoyl, alkyl)sulfamoyl, sulfamoylamino, mono(C₁₋₆ alkyl)sulfamoylamino, alkyl)sulfamoylamino, mono(C₁₋₆ alkyl)sulfamoyl(C₁₋₆ alkyl)amino and di(C₁₋₆ alkyl)sulfamoyl(C₁₋₆ alkyl)amino groups.

21. A compound as claimed in claim 20, wherein R^{1a} and R^{1b} are both hydrogen atoms, or a pharmaceutically acceptable salt thereof.

22. A compound as claimed in claim 20, wherein R^{2a} and R^{2b} are both hydrogen atoms, or a pharmaceutically acceptable salt thereof.

23. A compound as claimed in claim 20, wherein R^{2a} and R^{2b} form together —CH₂—CH₂—, or a pharmaceutically acceptable salt thereof.

24. A compound as claimed in claim 20, wherein R^{3a} and R^{3b} are both hydrogen atoms, or a pharmaceutically acceptable salt thereof.

25. A compound as claimed in claim 20, wherein Y is a hydrogen atom, or a pharmaceutically acceptable salt thereof.

26. A compound as claimed in claim 20, wherein Ar₂ is a group formed by removing two hydrogen atoms from benzene or pyridine, or a pharmaceutically acceptable salt thereof.

27. A compound as claimed in claim 20, wherein Ar₂ is 1,4-phenylenediyl, 3-methanesulfonylphenylene-1,4-diyl, pyridine-2,5-diyl or pyrimidine-2,5-diyl, or a pharmaceutically acceptable salt thereof.

28. A compound as claimed in claim 20, wherein Ar₁ is a 6-membered aromatic carbon ring group substituted with one or two fluorine atoms or chlorine atoms or a 6-membered nitrogen-containing aromatic heterocyclic group substituted with one or two fluorine atoms or chlorine atoms, or a pharmaceutically acceptable salt thereof.

29. A compound as claimed in claim 20, wherein Ar₁ is a phenyl group substituted with one or two fluorine atoms or chlorine atoms or a pyridyl group substituted with one or two fluorine atoms or chlorine atoms, or a pharmaceutically acceptable salt thereof.

30. A compound as claimed in claim 20, wherein Z is —OR⁸, or a pharmaceutically acceptable salt thereof.

31. A compound as claimed in claim 20, wherein Z is a hydroxy, methoxy, ethoxy, n-propyloxy, isopropyloxy, cyclopropyloxy, 2-fluoroethoxy, 2,2-difluoroethoxy, 2,2,2-trifluoroethoxy, 2-hydroxyethoxy, 2-hydroxypropyloxy, dimethylaminoethoxy, dimethylcarbamoylmethoxy, difluoromethoxy, 2-hydroxy-2-methylpropyloxy, cyanomethyloxy or methylsulfonyloxy group, or a pharmaceutically acceptable salt thereof.

32. A compound as claimed in claim 20, wherein the ring in the ring group A is a benzene, pyridine, pyridazine, pyrimidine, pyrazine, imidazo[1,2-a]pyridine, imidazo[1,2-a]pyrimidine, imidazo[1,2-a]pyrazine, imidazo[1,2-b]pyridazine, 1H-pyrrolo[2,3-c]pyridine, 2,3-dihydro-1,3-benzoxazole, [1,2,4]triazolo-[4,3-a]pyridine, [1,2,4]triazolo-[1,5-a]pyridine, pyrazolo[1,5-b]pyridazine, 2,3-dihydro-1H-pyrrolo[2,3-b]pyridine, 1,2-dehydropyridine, 1,2-dehydropyrimidine, 1,2-dihydropyrazine, indole, indoline, 1H-pyrazole or isothiazole ring, or a pharmaceutically acceptable salt thereof.

33. A compound as claimed in claim 32, wherein an optional substituent of the ring group A is nil or a halogen, C₁₋₆ alkylcarbonylamino, oxo or C₁₋₆ alkyl group, or a pharmaceutically acceptable salt thereof.

34. A compound as claimed in claim 20, wherein the ring group A is a 6-fluoropyridin-3-yl, 5-methylcarboxaminopyridin-3-yl, 1-oxidopyridin-3-yl, 1-oxidopyridin-4-yl, 1-methyl-1H-pyrazol-4-yl, 1-methyl-1,2-dehydropyridin-2-on-4-yl, 1-methyl-1,6-dehydropyridin-6-on-4-yl, 2-oxo-2,3-dihydro-1,3-benzoxazol-5-yl, imidazo[1,2-a]pyridin-7-yl, imidazo[1,2-a]pyrazin-3-yl, imidazo[1,2-a]pyrimidin-3-yl, 1-methyl-1H-pyrrolo[2,3-c]pyridin-3-yl, [1,2,4]triazolo[4,3-a]pyridin-7-yl, [1,2,4]triazolo[1,5-a]pyridin-7-yl or pyrazolo[1,5-b]pyridazin-3-yl group, or a pharmaceutically acceptable salt thereof.

35. A compound as claimed in claim 20, wherein the compound represented by the formula (I) is
N-{5-[1-({6-[(E)-(3,4-difluorophenyl)(methoxyimino)methyl]pyridin-3-yl}methyl)piperidin-4-yl]pyridin-3-yl}acetamide,
N-{5-[1-({6-[(E)-[(2,2-difluoroethoxy)imino](3,4-difluorophenyl)methyl]pyridin-3-yl}methyl)piperidin-4-yl]pyridin-3-yl}acetamide,
(E)-(3,4-difluorophenyl)(5-{[4-(1-methyl-1H-pyrazol-4-yl)piperidin-1-yl]methyl}pyridin-2-yl)methanone O-ethyloxime,
(E)-(3,4-difluorophenyl){5-[(4-imidazo[1,2-a]pyridin-7-yl-piperidin-1-yl)methyl]pyridin-2-yl}methanone O-ethyloxime,
5-[1-({6-(E)-(3,4-difluorophenyl)(ethoxyimino)methyl]pyridin-3-yl}methyl)piperidin-4-yl]-1,3-benzoxazole-2(3H)-one,
(E)-(3,4-difluorophenyl)(5-{[4-(1-methyl-1H-pyrrolo[2,3-c]pyridin-3-yl)-piperidin-1-yl]methyl}pyridin-2-yl)methanone O-ethyloxime,
(E)-(3,4-difluorophenyl) {5-[(4-imidazo[1,2-a]pyrazin-3-yl)-piperidin-1-yl]methyl}pyridin-2-yl}methanone O-ethyloxime,
(E)-(3,4-difluorophenyl)(5-{[4-(6-fluoropyridin-3-yl)piperidin-1-yl]methyl}pyridin-2-yl)methanone O-(2-hydroxy-2-methylpropyl)oxime,
(E)-(3,4-difluorophenyl){5-[(4-pyrazolo[1,5-b]pyridazin-3-ylpiperidin-1-yl)methyl]pyridin-2-yl}methanone O-(2-hydroxy-2-methylpropyl)oxime,
(E)-(3,4-difluorophenyl)(5-{[4-(6-fluoropyridin-3-yl)piperidin-1-yl]methyl}pyridin-2-yl)methanone 0-[(methylsulfonyl)methyl]oxime,
(E)-(3,4-difluorophenyl){5-[(4-[1,2,4]triazolo[4,3-a]pyridin-7-ylpiperidin-1-yl)methyl]pyridin-2-yl}methanone O-cyclopropyloxime,
{[((1E)-(3,4-difluorophenyl){5-[(4-[1,2,4-triazolo[1,5-a]pyridin-7-ylpiperidin-1-yl)methyl]pyridin-2-yl}methylene)amino]oxy}acetonitrile,
5-[1-(1-{6-[(E)-(3,4-difluorophenyl)(ethoxyimino)methyl]pyridin-3-yl}ethyl)piperidin-4-yl]-1-methylpyridin-2(1H)-one,
4-[1-({5-[(E))-(3,4-difluorophenyl)(ethoxyimino)methyl]pyridin-2-yl}methyl)piperidin-4-yl]-1-methylpyridin-2(1H)-one,
(E)-(3,4-difluorophenyl)(6-{[4-(1-oxidopyridin-3-yl)piperidin-1-yl]methyl}pyridin-3-yl)methanone O-ethyloxime,
(Z)-(3,4-difluorophenyl) {4-[(4-imidazo[1,2-a]pyrimidin-3-ylpiperidin-1-yl)methyl]phenyl}methanone O-(2-hydroxy-2-methylpropyl)oxime,
({[(1Z)-(3,4-difluorophenyl)(4-{[4-(1-oxidopyridin-4-yl)piperidin-1-yl]methyl}phenyl)methylene]amino}oxy)acetonitrile,
5-(1-{4-[(Z)-(5-chloropyridin-2-yl)(ethoxyimino)methyl]benzyl}piperidin-4-yl]-1-methylpyridin-2(1H)-one,
(Z)-(5-chloropyridin-2-yl) {[(4-[1,2,4]triazolo[4,3-a]pyridin-7-ylpiperidin-1-yl)methyl]phenyl}methanone O-ethyloxime,
(E)-(3,4-difluorophenyl)(5-{[4-(6-fluoropyridin-3-yl)piperidin-1-yl]methyl}pyrimidin-2-yl)methanone O-cyclopropyloxime or
({[(1E)-(3,4-difluorophenyl)(5-{[4-(6-fluoropyridin-3-yl)piperidin-1-yl]methyl}pyridin-2-yl)methylene]amino}oxy)acetonitrile,
or a pharmaceutically acceptable salt thereof.

36. A melanin-concentrating hormone receptor antagonist which comprises a compound as claimed in claim 20 or a pharmaceutically acceptable salt thereof as the active ingredient.

37. A pharmaceutical composition which contains a pharmaceutically acceptable additive and a compound as claimed in claim 20 or a pharmaceutically acceptable salt thereof.

38. A method of treating obesity, diabetes, fatty liver, bulimia, depression or anxiety which comprises administering a compound as claimed in claim 20 or a pharmaceutically acceptable salt.
